(19) United States
(12) Patent Application Publication
(10) Pub. No.: US 2025/0241199 A1
(43) Pub. Date: Jul. 24, 2025

(54) COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Youngkyoung JO, Suwon-si (KR); Ho Kuk JUNG, Suwon-si (KR); Mijin LEE, Suwon-si (KR); Eunhye AN, Suwon-si (KR); Giwook KANG, Suwon-si (KR); Eunjeong CHOI, Suwon-si (KR); Dongjin JANG, Suwon-si (KR)

(21) Appl. No.: 19/021,465

(22) Filed: Jan. 15, 2025

(30) Foreign Application Priority Data
Jan. 22, 2024 (KR) 10-2024-0009468

(51) Int. Cl. H10K 85/60; C09K 11/02; C09K 11/06; H10K 50/12; H10K 50/15; H10K 85/30

(52) U.S. Cl. CPC H10K 85/6572; C09K 11/02; C09K 11/06; H10K 85/346; H10K 85/654; H10K 85/6574; H10K 85/658; C09K 2211/1007; C09K 2211/1014; C09K 2211/1022; C09K 2211/1029; C09K 2211/1044; H10K 50/121; H10K 50/156

(57) ABSTRACT

A composition for an organic optoelectronic device, an organic optoelectronic device including the same, and a display device, the composition for an organic optoelectronic device including a first compound represented by Chemical Formula 1 and a second compound represented by Chemical Formula 2,

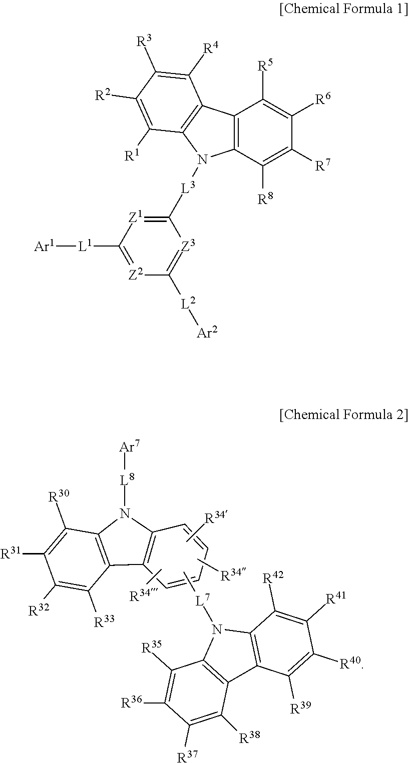

### CROSS-REFERENCE TO RELATED APPLICATION

This application claims priority to and the benefit of Korean Patent Application No. 10-2024-0009468 filed in the Korean Intellectual Property Office on Jan. 22, 2024, the entire contents of which are incorporated herein by reference.

### BACKGROUND

### 1. Field

Embodiments relate to a composition for an organic optoelectronic device, an organic optoelectronic device, and a display device.

### 2. Description of the Related Art

An organic optoelectronic device (e.g., organic optoelectronic diode) is a device capable of converting electrical energy and optical energy to each other.

Organic optoelectronic devices may be largely divided into two types according to a principle of operation. One may be a photoelectric device that generates electrical energy by separating excitons formed by light energy into electrons and holes, and transferring the electrons and holes to different electrodes, respectively and the other may be a light emitting device that generates light energy from electrical energy by supplying voltage or current to the electrodes.

### SUMMARY

Embodiments are directed to a composition for an organic optoelectronic device, the composition including a first compound represented by Chemical Formula 1; and a second compound represented by Chemical Formula 2:

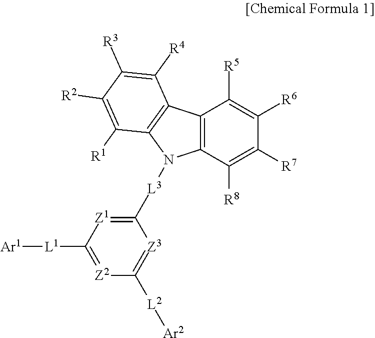

wherein, in Chemical Formula 1, Z¹ to Z³ are each independently N or CR^{a}, provided that at least two of Z¹ to Z³ are N, L¹ to L³ are each independently a single bond, a substituted or unsubstituted C6 to C20 arylene group or a substituted or unsubstituted divalent C2 to C20 heterocyclic group, one of Ar¹ and Ar² is a substituted or unsubstituted carbazolyl group, the other of Ar¹ and Ar² is a substituted or unsubstituted C6 to C30 aryl group, R¹ to R⁸ and R^{a} are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, and R¹ to R⁸ are each separately present or two adjacent ones are linked to form a ring,

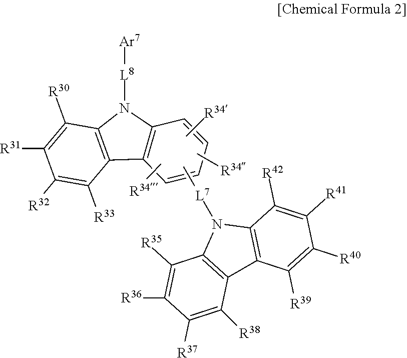

in Chemical Formula 2, L⁷ and L¹ are each independently a single bond or a substituted or unsubstituted C6 to C30 arylene group, Ar⁷ is a substituted or unsubstituted C6 to C30 aryl group or a substituted or unsubstituted C2 to C30 heterocyclic group, and R³⁰ to R³³, R^{34′}, R^{34″}, R^{34′″}, and R³⁵ to R⁴² are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen.

The embodiments may be realized by providing an organic optoelectronic device including an anode and a cathode facing each other; and a light emitting layer between the anode and the cathode; wherein the light emitting layer includes the composition for an organic optoelectronic device according to an embodiment.

The embodiments may be realized by providing a display device comprising the organic optoelectronic device according to an embodiment.

### BRIEF DESCRIPTION OF THE DRAWINGS

Features will become apparent to those of skill in the art by describing in detail exemplary embodiments with reference to the attached drawings in which:

FIG. 1 is a cross-sectional view showing an example of an organic light emitting diode, which is an example of an organic optoelectronic device according to an embodiment, and

FIG. 2 is a cross-sectional view showing another example of an organic light emitting diode, which is an example of an organic optoelectronic device according to another embodiment.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout. As used herein, the term “or” is not an exclusive term, e.g., “A or B” would include A, B, or A and B.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In some embodiments, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In some embodiments, “substituted” refers to replacement of at least one hydrogen of a substituent or a compound by deuterium, a C1 to C20 alkyl group, a C6 to C30 aryl group, or a cyano group. In some embodiments “substituted” refers to replacement of at least one hydrogen of a substituent or a compound by deuterium, a C1 to C5 alkyl group, a C6 to C18 aryl group, or a cyano group. In some embodiments, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

In the present specification, “hydrogen substitution (-H)” may include “deuterium substitution (-D)” or “tritium substitution (-T).” For example, any hydrogen in any compound described herein may be protium, deuterium, or tritium (e.g., based on natural or artificial substitution).

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, e.g., a phenyl group, a naphthyl group, or the like, two or more hydrocarbon aromatic moieties may be linked by a sigma bond and may be, e.g., a biphenyl group, a terphenyl group, a quarterphenyl group, or the like, and two or more hydrocarbon aromatic moieties may be fused directly or indirectly to provide a non-aromatic fused ring, e.g., a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound, e.g., aryl group, a cycloalkyl group, a fused ring thereof, or a combination thereof. If the heterocyclic group is a fused ring, the entire ring or each ring of the heterocyclic group may include one or more heteroatoms.

In an implementation, “heteroaryl group” may refer to aryl group including at least one heteroatom selected from N, O, S, P, and Si. Two or more heteroaryl groups may be linked by a sigma bond directly, or if the heteroaryl group includes two or more rings, the two or more rings may be fused. If the heteroaryl group is a fused ring, each ring may include one to three heteroatoms.

In an implementation, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof.

In an implementation, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof.

As used herein, hole characteristics refer to an ability to donate an electron to form a hole if an electric field is applied and that a hole formed in the anode may be easily injected into the light emitting layer and transported in the light emitting layer due to conductive characteristics according to a highest occupied molecular orbital (HOMO) level.

In addition, electronic characteristics refer to an ability to accept an electron if an electric field is applied and that electron formed in the cathode may be easily injected into the light emitting layer and transported in the light emitting layer due to conductive characteristics according to a lowest unoccupied molecular orbital (LUMO) level.

Hereinafter, a composition for an organic optoelectronic device according to some embodiments will be described.

The composition for an organic optoelectronic device according to some embodiments may include, e.g., a first compound represented by Chemical Formula 1 and a second compound represented by Chemical Formula 2.

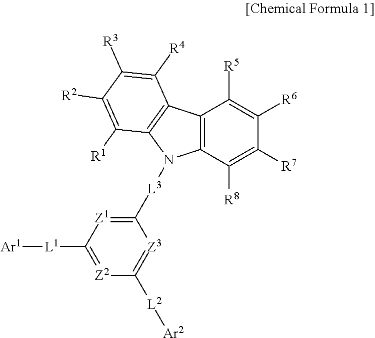

In Chemical Formula 1, Z¹ to Z³ may each independently be, e.g., N or CR.

In an implementation, least two of Z¹ to Z³ may be, e.g., N.

L¹ to L³ may each independently be or include, e.g., a single bond, a substituted or unsubstituted C6 to C20 arylene group, or a substituted or unsubstituted divalent C2 to C20 heterocyclic group.

One of Ar¹ and Ar² may be or include, e.g., a substituted or unsubstituted carbazolyl group.

The other of Ar¹ and Ar² may be or include, e.g., a substituted or unsubstituted C6 to C30 aryl group.

R¹ to R⁸ and R^{a} may each independently be or include, e.g., hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen.

R¹ to R⁸ may each be separately present or two adjacent ones may be linked to form a ring.

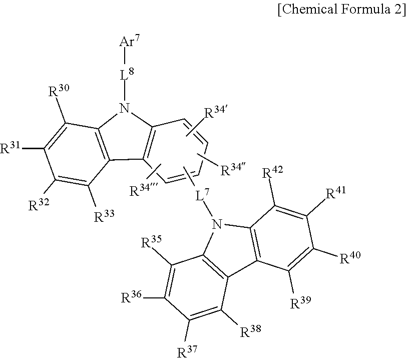

In Chemical Formula 2, L⁷ and L⁸ may each independently be or include, e.g., a single bond or a substituted or unsubstituted C6 to C30 arylene group.

Ar⁷ may be or include, e.g., a substituted or unsubstituted C6 to C30 aryl group or a substituted or unsubstituted C2 to C30 heterocyclic group.

R³⁰ to R³³, R^{34′}, R^{34″}, R^{34′″}, and R³⁵ to R⁴² may each independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen.

The first compound and the second compound may be bipolar compounds having both electron transport characteristics and hole transport characteristics, respectively. The first compound may be a bipolar compound with relatively strong electron transport characteristics, and the second compound may be a bipolar compound with relatively strong hole transport characteristics. In an implementation, the first compound and the second compound may exhibit good interfacial characteristics due to their structures.

The composition for an organic optoelectronic device may include the first compound and the second compound together to finely control the mobility of holes and electrons to balance holes and electrons in the active layer (e.g., light emitting layer) of the organic optoelectronic device.

In an implementation, the composition for an organic optoelectronic device may be used as a host for a light emitting layer, and may have good electrical matching with a blue light-emitting dopant that emits light in a blue emission spectrum described later, thereby increasing efficiency of the organic optoelectronic device and suppressing deterioration of the organic optoelectronic device. In an implementation, at least one of the first compound and the second compound may have a high triplet energy level of greater than or equal to about 2.8 eV, so that exciton transfer to the blue light-emitting dopant may be facilitated, thereby implementing an organic optoelectronic device having high efficiency and long life-span.

In an implementation, in Chemical Formula 1, Z¹ to Z³ may each be N.

In an implementation, in Chemical Formula 1, Z¹ and Z² may each be N and Z³ may be CR.

In an implementation, in Chemical Formula 1, Z¹ and Z³ may each be N and Z² may be CR^{a}.

In an implementation, in Chemical Formula 1, Z² and Z³ may each be N and Z¹ may be CR^{a}.

In an implementation, in Chemical Formula 1, L¹ to L³ may each independently be, e.g., a single bond, a substituted or unsubstituted phenylene group, a substituted or unsubstituted biphenylene group, a substituted or unsubstituted terphenylene group or a substituted or unsubstituted naphthylene group, a substituted or unsubstituted benzofuranylene group, a substituted or unsubstituted dibenzofuranylene group, a substituted or unsubstituted benzothiophenylene group, a substituted or unsubstituted dibenzothiphenylene group, or a substituted or unsubstituted fluorenylene group.

In an implementation, in Chemical Formula 1, L¹ and L² may each independently be, e.g., a single bond or a substituted or unsubstituted C6 to C20 arylene group and in Chemical Formula 1, L³ may be a single bond. In an implementation, in Chemical Formula 1, L¹ and L² may each independently be, e.g., a single bond or a substituted or unsubstituted phenylene group, a substituted or unsubstituted biphenylene group, a substituted or unsubstituted terphenylene group or a substituted or unsubstituted naphthylene group, and in Chemical Formula 1, L³ may be, e.g., a single bond.

In an implementation, in Chemical Formula 1, Ar¹ may be, e.g., a substituted or unsubstituted C6 to C30 aryl group and Ar² may be a substituted or unsubstituted carbazolyl group. In an implementation, Ar¹ may be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, or a substituted or unsubstituted triphenylene group.

In an implementation, in Chemical Formula 1, if Ar² is a substituted or unsubstituted carbazolyl group, L² and L³ may be different from each other. In an implementation, one of L² and L³ may be, e.g., a single bond and the other of L² and L³ may be, e.g., a substituted or unsubstituted C6 to C20 arylene group, e.g., a substituted or unsubstituted phenylene group, a substituted or unsubstituted biphenylene group, a substituted or unsubstituted terphenylene group, or a substituted or unsubstituted naphthylene group.

In an implementation, in Chemical Formula 1, R^{a} may be, e.g., hydrogen, deuterium, a substituted or unsubstituted methyl group, a substituted or unsubstituted ethyl group, a substituted or unsubstituted phenyl group, or a cyano group.

The first compound may be, e.g., represented by Chemical Formula 1a.

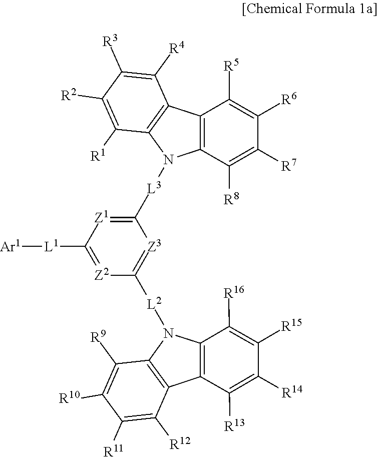

In Chemical Formula 1a, Z¹ to Z³, L¹ to L³ and R¹ to R⁸ may be defined the same as those of Chemical Formula 1.

Ar¹ may be, e.g., a substituted or unsubstituted C6 to C30 aryl group, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, or a substituted or unsubstituted triphenylene group.

R⁹ to R¹⁶ may each independently be or include, e.g., hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, and R⁹ to R¹⁶ may each be separately present or two adjacent ones may be linked to form a ring.

In an implementation, in Chemical Formula 1a, L² and L³ may be different from each other, e.g., L² may be a substituted or unsubstituted phenylene group, a substituted or unsubstituted biphenylene group, a substituted or unsubstituted terphenylene group, or a substituted or unsubstituted naphthylene group and L³ may be a single bond.

In an implementation, at least one of each substituent in Chemical Formula 1 and 1a may be substituted with deuterium. The number of substituted deuterium atoms may be 1 to the maximum number of hydrogens in the formula, e.g., 1 to 40 or 1 to 30.

In an implementation, the second compound may be represented by one of Chemical Formulae 2a to 2d.

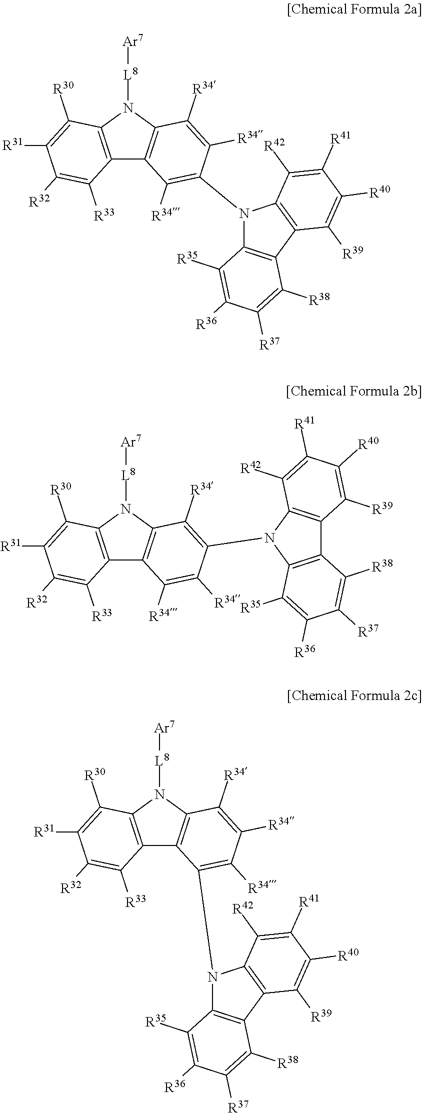

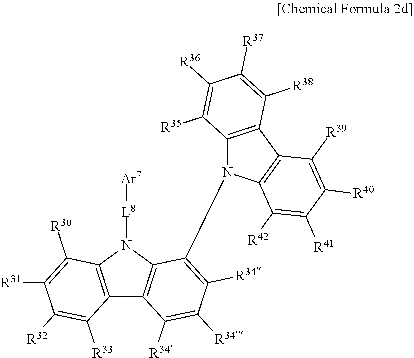

In Chemical Formulae 2a to 2d, L⁸, Ar⁷, R³⁰ to R³³, R^{34′}, R^{34″}, R^{34′″}, and R³⁵ to R⁴² may be defined the same as those of Chemical Formula 2.

In an implementation, in Chemical Formulae 2, and 2a to 2d, L⁸ may be, e.g., a single bond, a substituted or unsubstituted phenylene group, a substituted or unsubstituted biphenylene group, or a substituted or unsubstituted terphenylene group.

In an implementation, in Chemical Formulae 2, and 2a to 2d, Ar⁷ may be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylene group, a substituted or unsubstituted carbazolyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, a substituted or unsubstituted fluorenyl group, or a substituted or unsubstituted silyl group.

In an implementation, in Chemical Formulae 2, and 2a to 2d, R³⁰ to R³³, R^{34′}, R^{34″}, R^{34′″}, and R³⁵ to R⁴² may each independently be, e.g., hydrogen, deuterium, a substituted or unsubstituted methyl group, a substituted or unsubstituted ethyl group, a substituted or unsubstituted phenyl group, a substituted or unsubstituted biphenyl group, a substituted or unsubstituted carbazolyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, a substituted or unsubstituted fluorenyl group, a substituted or unsubstituted silyl group, or a cyano group.

In an implementation, at least one of Ar⁷, R³⁰ to R³³, R^{34′}, R^{34″}, R^{34′″}, and R³⁵ to R⁴² may be, e.g., a substituted or unsubstituted carbazolyl group.

In an implementation, in Chemical Formulae 2, and 2a to 2d, at least one of each substituent may be substituted with deuterium. The number of substituted deuterium atoms may be 1 to the maximum number of hydrogens in the formula, e.g., 1 to 40 or 1 to 30.

In an implementation, the first compound may include, e.g., a compound of Group 1.

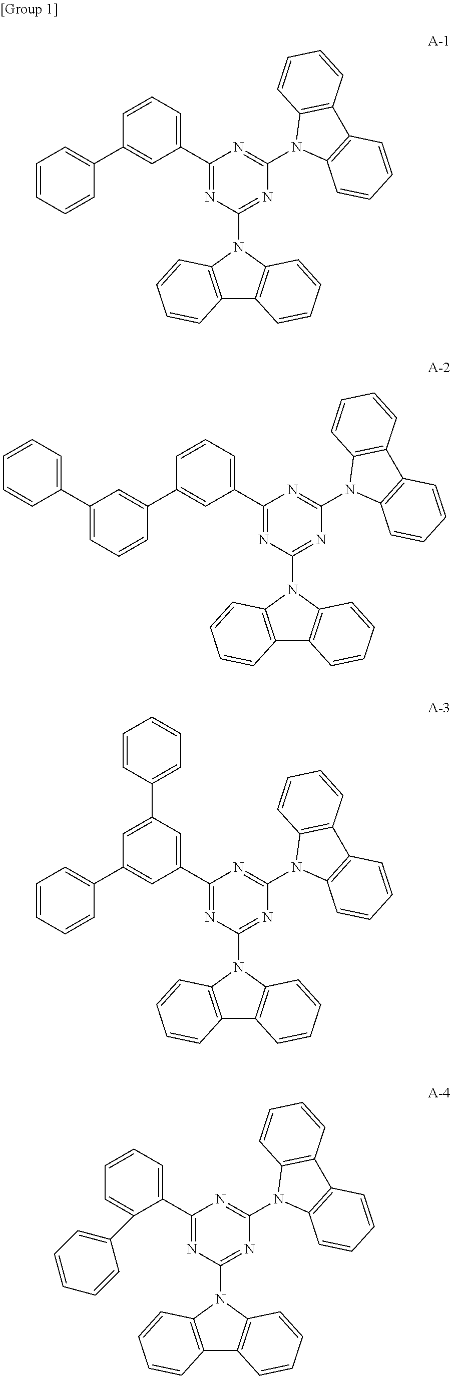 [SMILES: c1ccc(-c2cccc(-c3nc(-n4c5ccccc5c5ccccc54)nc(-n4c5ccccc5c5ccccc54)n3)c2)cc1 ; c1ccc(-c2cccc(-c3cccc(-c4nc(-n5c6ccccc6c6ccccc65)nc(-n5c6ccccc6c6ccccc65)n4)c3)c2)cc1 ; c1ccc(-c2cc(-c3ccccc3)cc(-c3nc(-n4c5ccccc5c5ccccc54)nc(-n4c5ccccc5c5ccccc54)n3)c2)cc1 ; c1ccc(-c2ccccc2-c2nc(-n3c4ccccc4c4ccccc43)nc(-n3c4ccccc4c4ccccc43)n2)cc1]

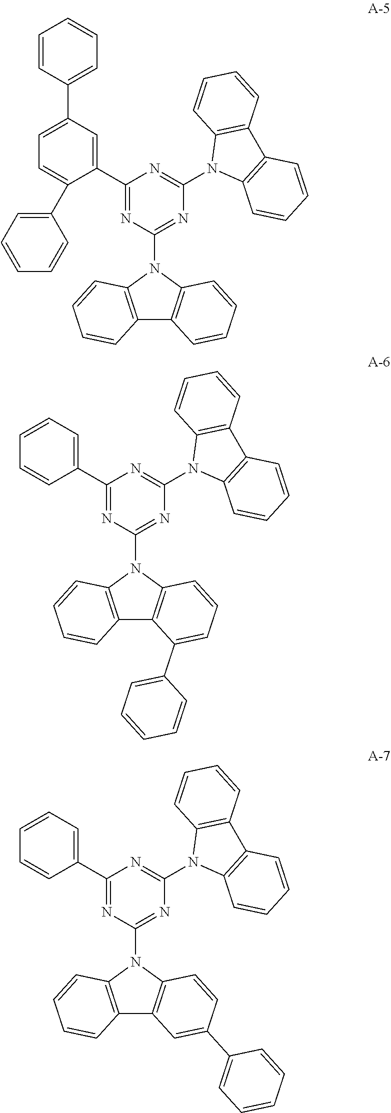

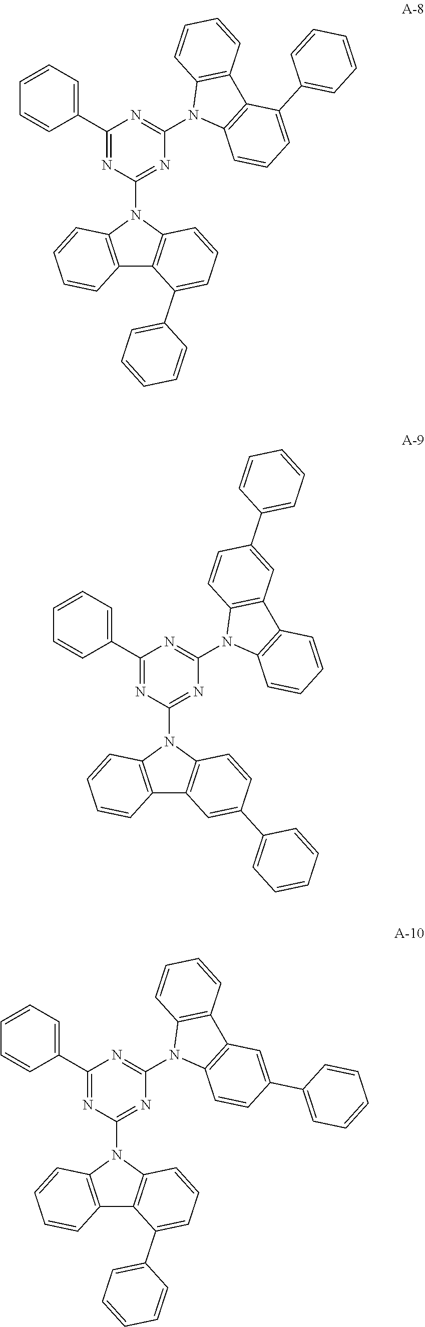 [SMILES: c1ccc(-c2nc(-n3c4ccccc4c4c(-c5ccccc5)cccc43)nc(-n3c4ccccc4c4c(-c5ccccc5)cccc43)n2)cc1 ; c1ccc(-c2ccc3c(c2)c2ccccc2n3-c2nc(-c3ccccc3)nc(-n3c4ccccc4c4cc(-c5ccccc5)ccc43)n2)cc1 ; c1ccc(-c2ccc3c(c2)c2ccccc2n3-c2nc(-c3ccccc3)nc(-n3c4ccccc4c4c(-c5ccccc5)cccc43)n2)cc1]

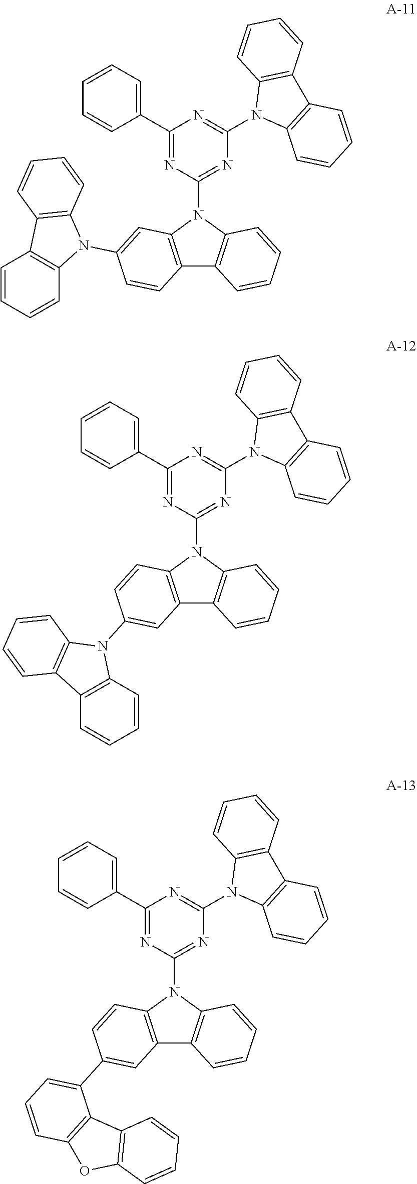 [SMILES: c1ccc(-c2nc(-n3c4ccccc4c4ccccc43)nc(-n3c4ccccc4c4ccc(-n5c6ccccc6c6ccccc65)cc43)n2)cc1 ; c1ccc(-c2nc(-n3c4ccccc4c4ccccc43)nc(-n3c4ccccc4c4cc(-n5c6ccccc6c6ccccc65)ccc43)n2)cc1 ; c1ccc(-c2nc(-n3c4ccccc4c4ccccc43)nc(-n3c4ccccc4c4cc(-c5cccc6oc7ccccc7c56)ccc43)n2)cc1]

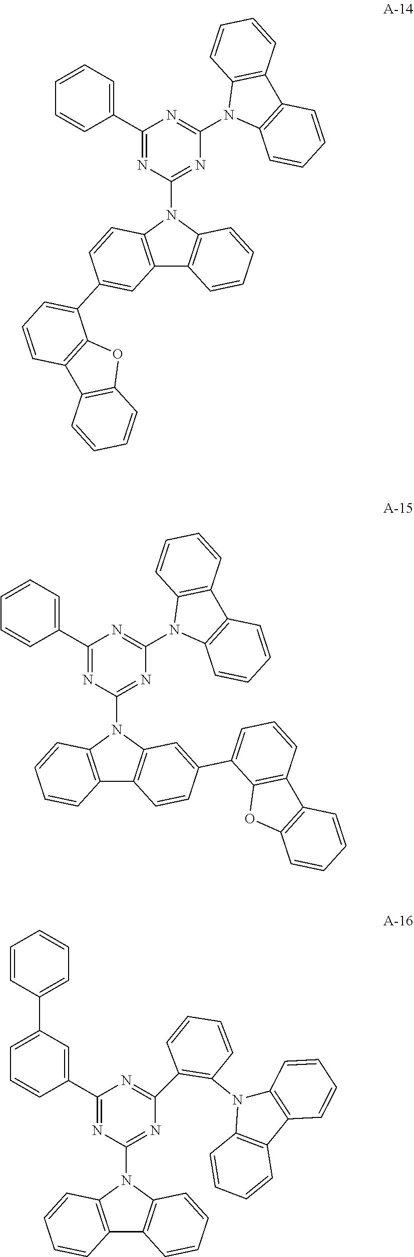 [SMILES: c1ccc(-c2nc(-n3c4ccccc4c4ccccc43)nc(-n3c4ccccc4c4cc(-c5cccc6c5oc5ccccc56)ccc43)n2)cc1 ; c1ccc(-c2nc(-n3c4ccccc4c4ccccc43)nc(-n3c4ccccc4c4ccc(-c5cccc6c5oc5ccccc56)cc43)n2)cc1 ; c1ccc(-c2cccc(-c3nc(-c4ccccc4-n4c5ccccc5c5ccccc54)nc(-n4c5ccccc5c5ccccc54)n3)c2)cc1]

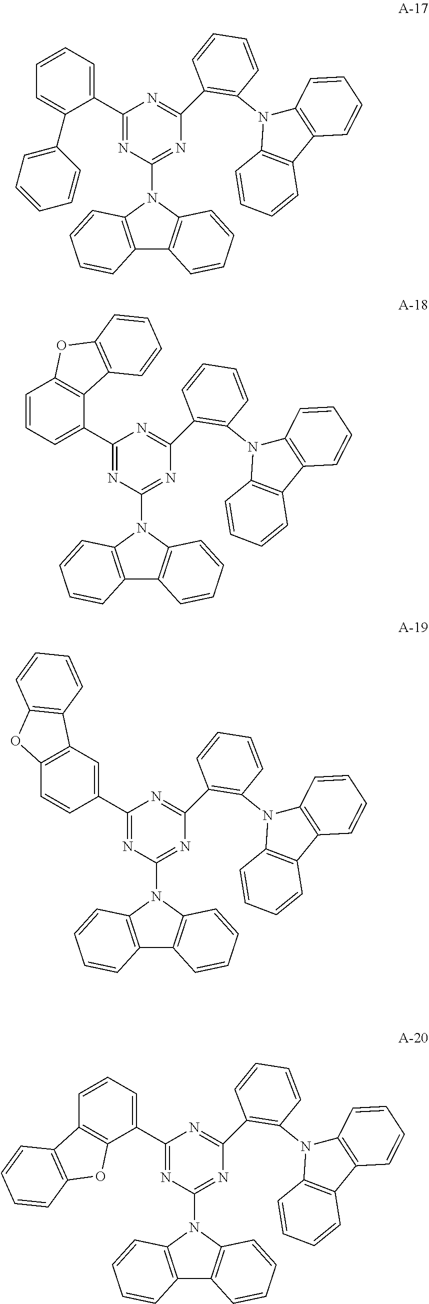

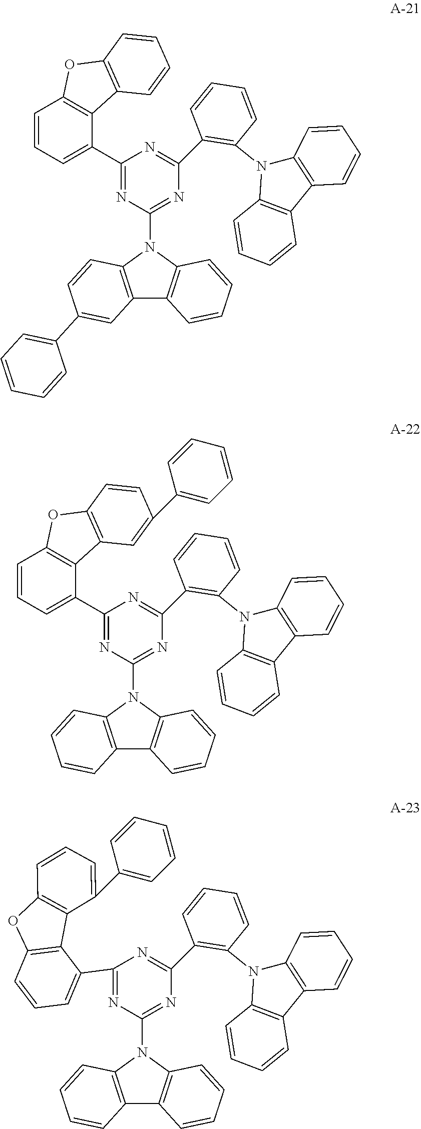 [SMILES: c1ccc(-c2ccc3c(c2)c2ccccc2n3-c2nc(-c3ccccc3-n3c4ccccc4c4ccccc43)nc(-c3cccc4oc5ccccc5c34)n2)cc1 ; c1ccc(-c2ccc3oc4cccc(-c5nc(-c6ccccc6-n6c7ccccc7c7ccccc76)nc(-n6c7ccccc7c7ccccc76)n5)c4c3c2)cc1 ; c1ccc(-c2cccc3oc4cccc(-c5nc(-c6ccccc6-n6c7ccccc7c7ccccc76)nc(-n6c7ccccc7c7ccccc76)n5)c4c23)cc1]

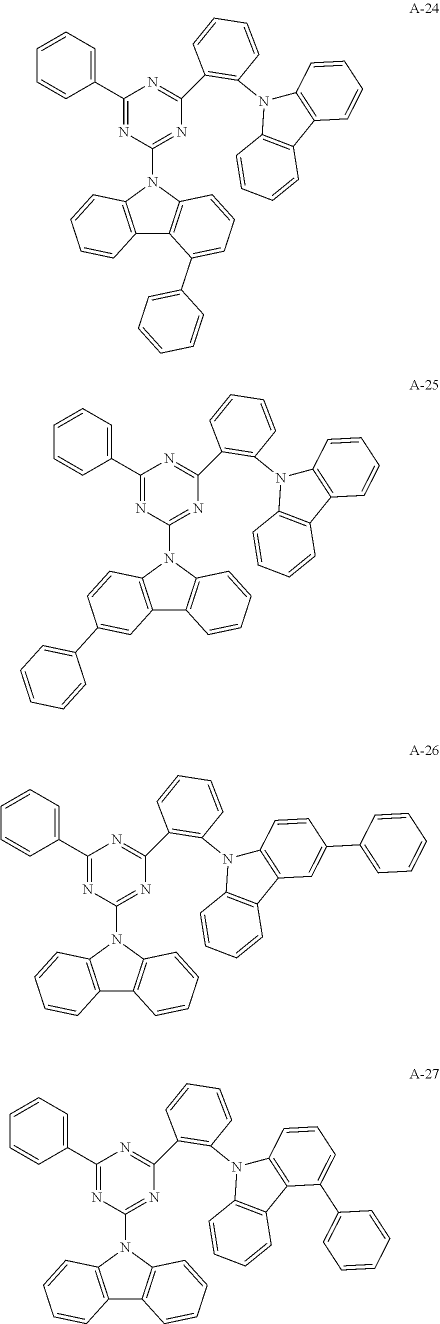 [SMILES: c1ccc(-c2nc(-c3ccccc3-n3c4ccccc4c4ccccc43)nc(-n3c4ccccc4c4c(-c5ccccc5)cccc43)n2)cc1 ; c1ccc(-c2ccc3c(c2)c2ccccc2n3-c2nc(-c3ccccc3)nc(-c3ccccc3-n3c4ccccc4c4ccccc43)n2)cc1 ; c1ccc(-c2ccc3c(c2)c2ccccc2n3-c2ccccc2-c2nc(-c3ccccc3)nc(-n3c4ccccc4c4ccccc43)n2)cc1 ; c1ccc(-c2nc(-c3ccccc3-n3c4ccccc4c4c(-c5ccccc5)cccc43)nc(-n3c4ccccc4c4ccccc43)n2)cc1]

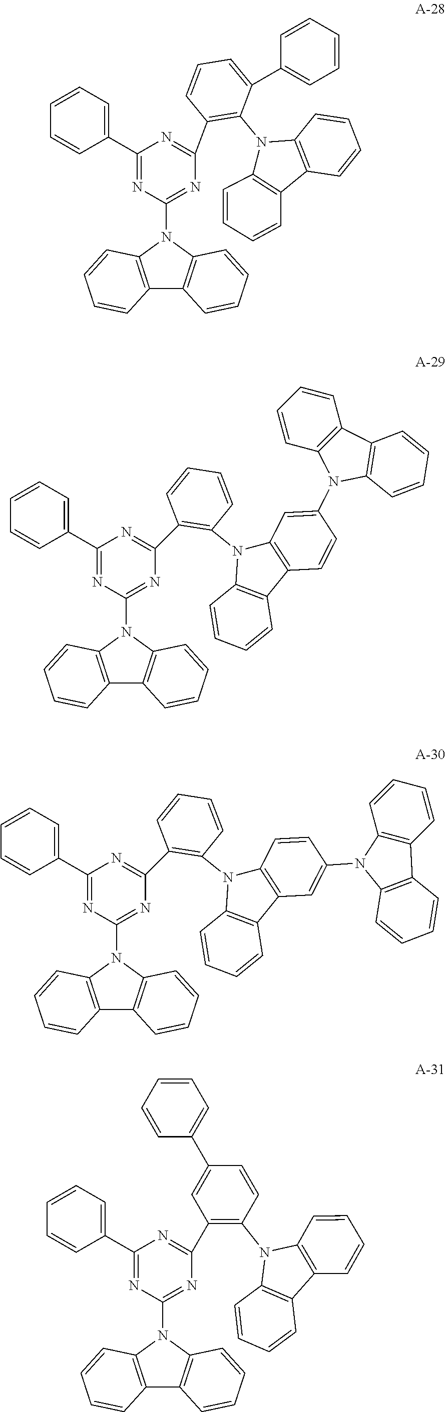

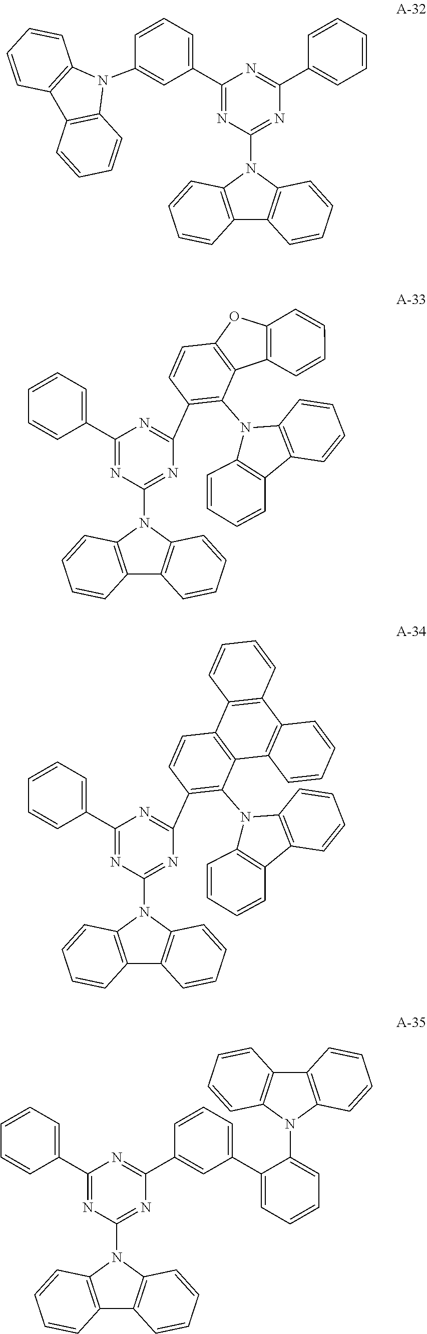 [SMILES: c1ccc(-c2nc(-c3cccc(-n4c5ccccc5c5ccccc54)c3)nc(-n3c4ccccc4c4ccccc43)n2)cc1 ; c1ccc(-c2nc(-c3ccc4oc5ccccc5c4c3-n3c4ccccc4c4ccccc43)nc(-n3c4ccccc4c4ccccc43)n2)cc1 ; c1ccc(-c2nc(-c3ccc4c5ccccc5c5ccccc5c4c3-n3c4ccccc4c4ccccc43)nc(-n3c4ccccc4c4ccccc43)n2)cc1 ; c1ccc(-c2nc(-c3cccc(-c4ccccc4-n4c5ccccc5c5ccccc54)c3)nc(-n3c4ccccc4c4ccccc43)n2)cc1]

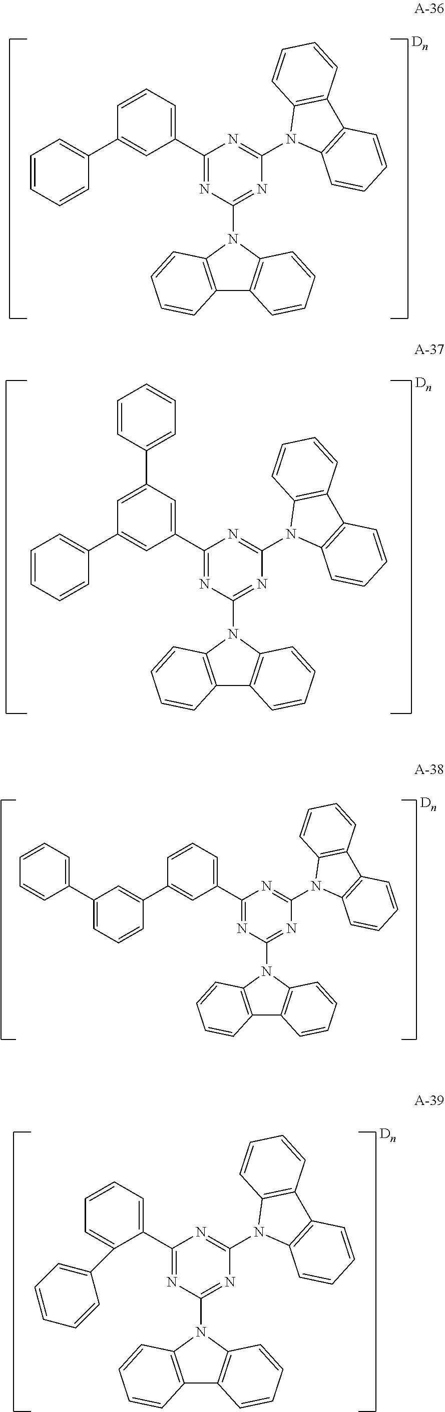

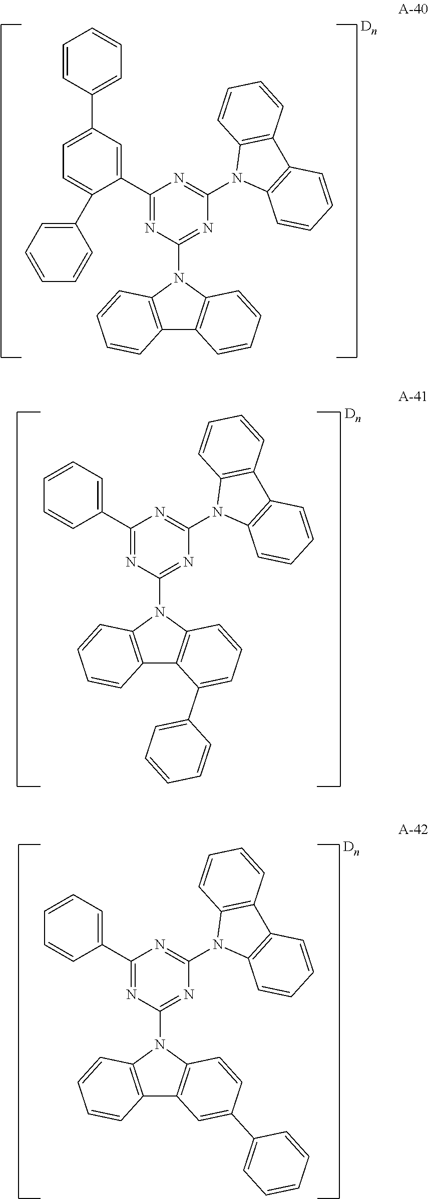

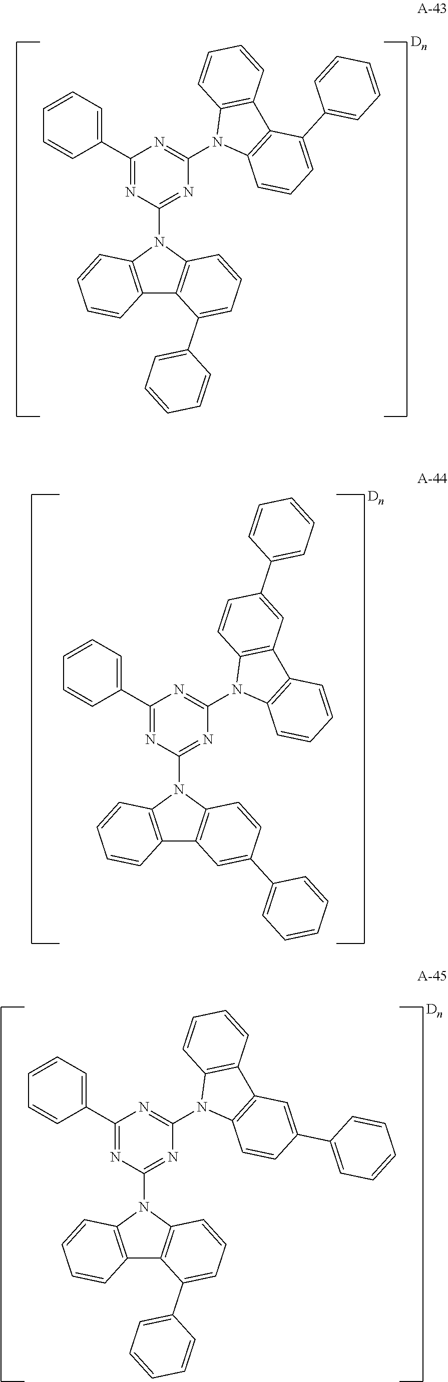

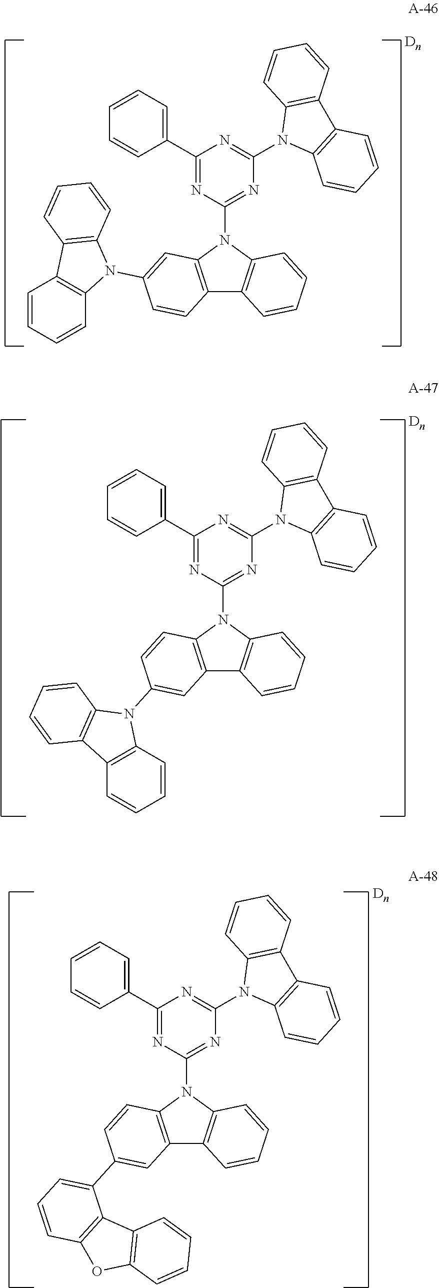

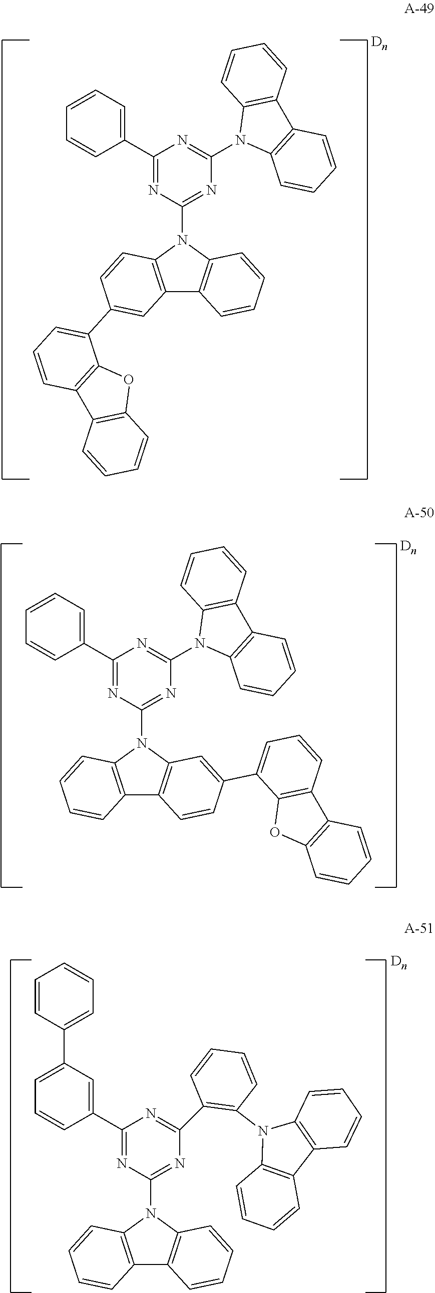

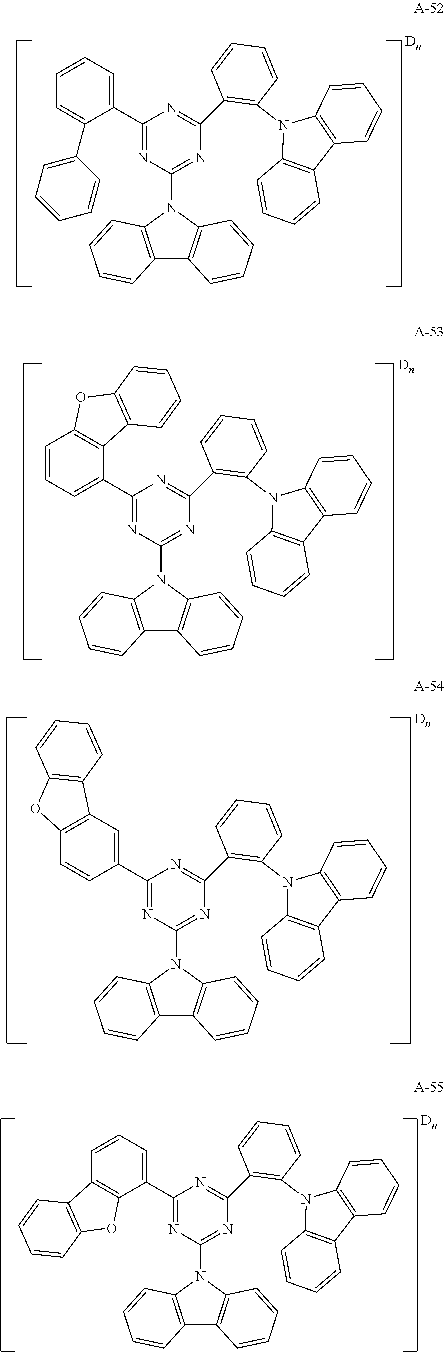

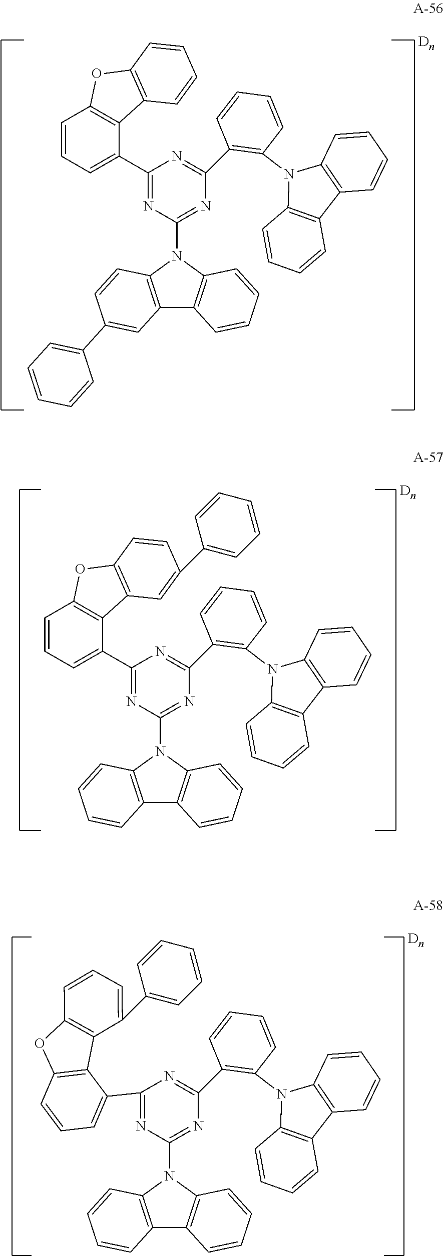

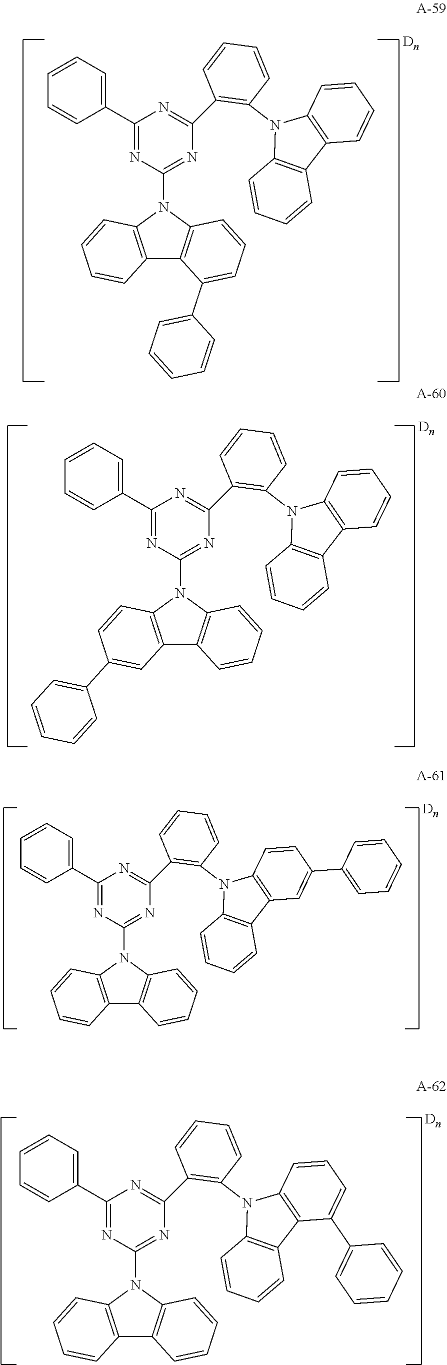

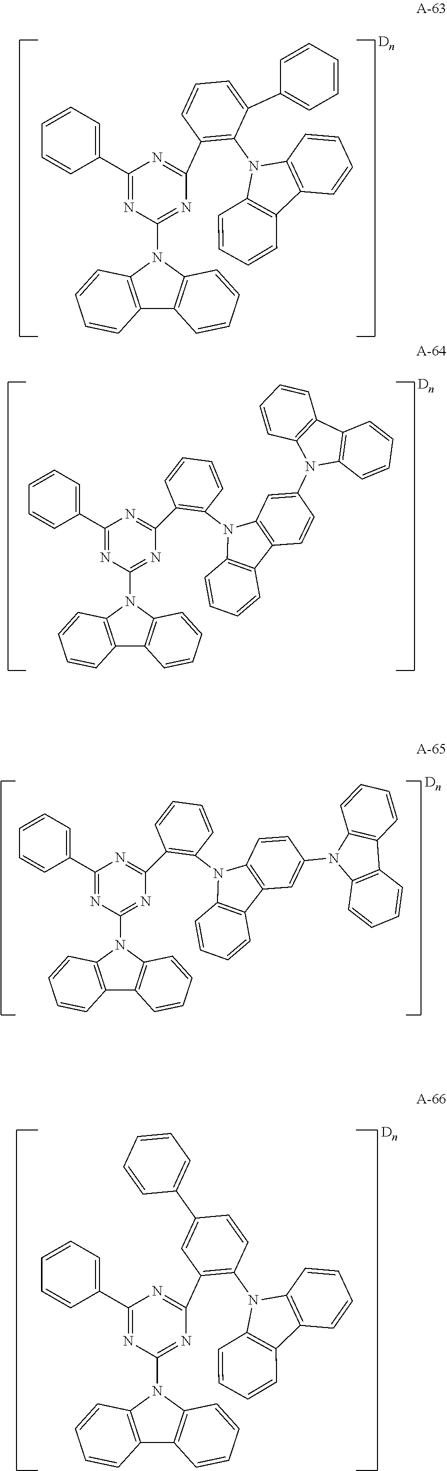

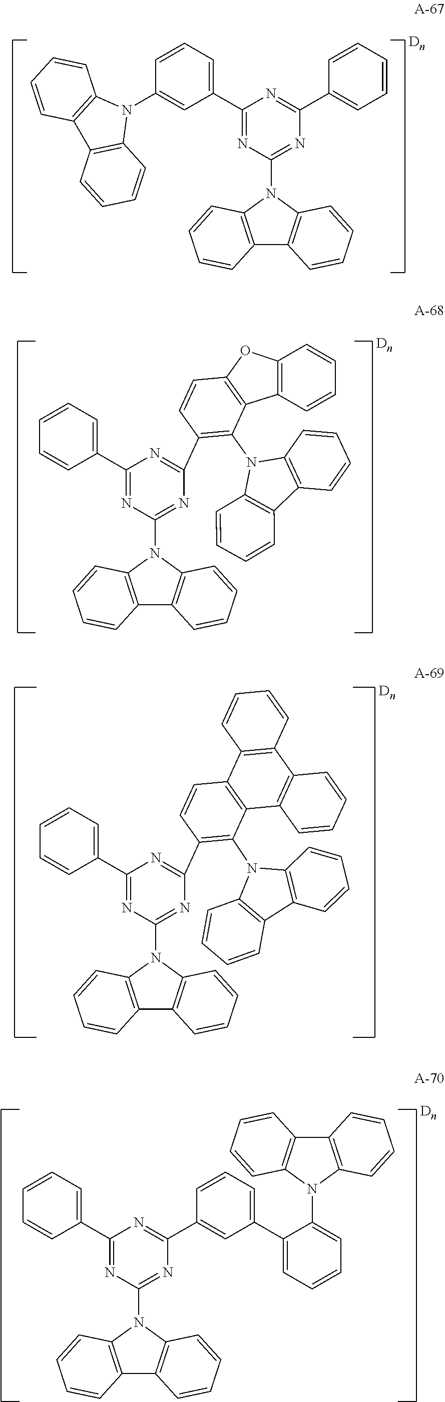

(Dn indicates the number of hydrogen atoms replaced by deuterium, and indicates a structure in which one or more deuterium atoms are substituted.) However, as noted above, any hydrogen in any compound may be protium, deuterium, or tritium, based on natural or artificial substitution.

In an implementation, the second compound may include, e.g., a compound of Group 2.

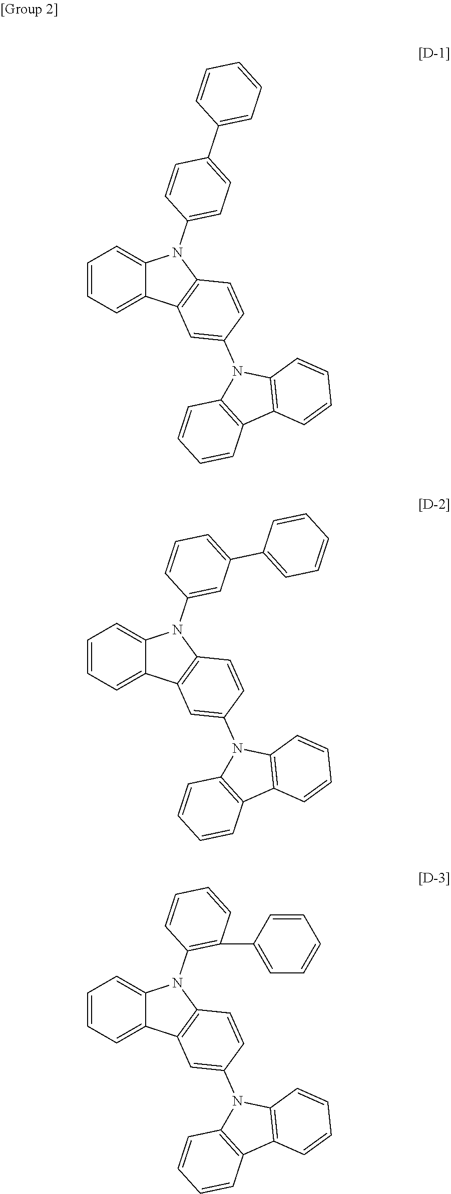 [SMILES: c1ccc(-c2ccc(-n3c4ccccc4c4cc(-n5c6ccccc6c6ccccc65)ccc43)cc2)cc1 ; c1ccc(-c2cccc(-n3c4ccccc4c4cc(-n5c6ccccc6c6ccccc65)ccc43)c2)cc1 ; c1ccc(-c2ccccc2-n2c3ccccc3c3cc(-n4c5ccccc5c5ccccc54)ccc32)cc1]

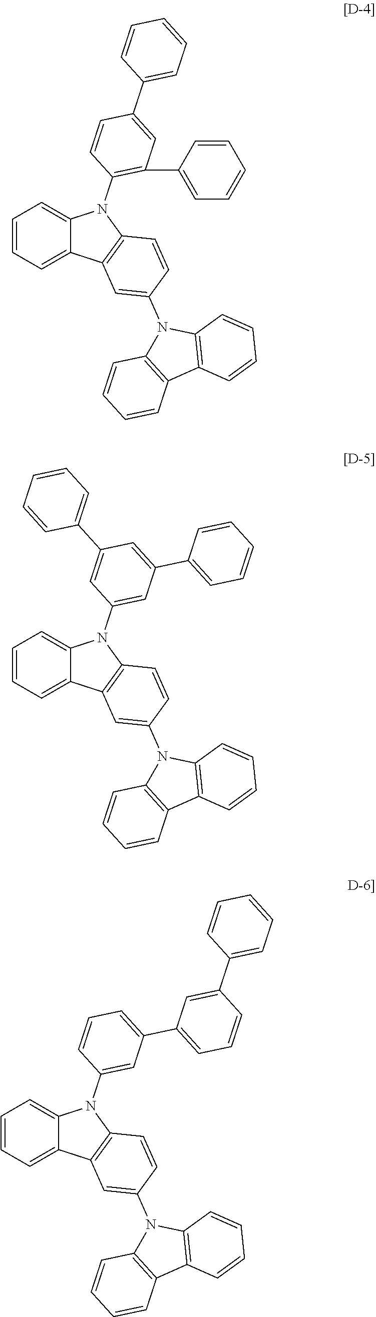 [SMILES: c1ccc(-c2ccc(-n3c4ccccc4c4cc(-n5c6ccccc6c6ccccc65)ccc43)c(-c3ccccc3)c2)cc1 ; c1ccc(-c2cc(-c3ccccc3)cc(-n3c4ccccc4c4cc(-n5c6ccccc6c6ccccc65)ccc43)c2)cc1 ; c1ccc(-c2cccc(-c3cccc(-n4c5ccccc5c5cc(-n6c7ccccc7c7ccccc76)ccc54)c3)c2)cc1]

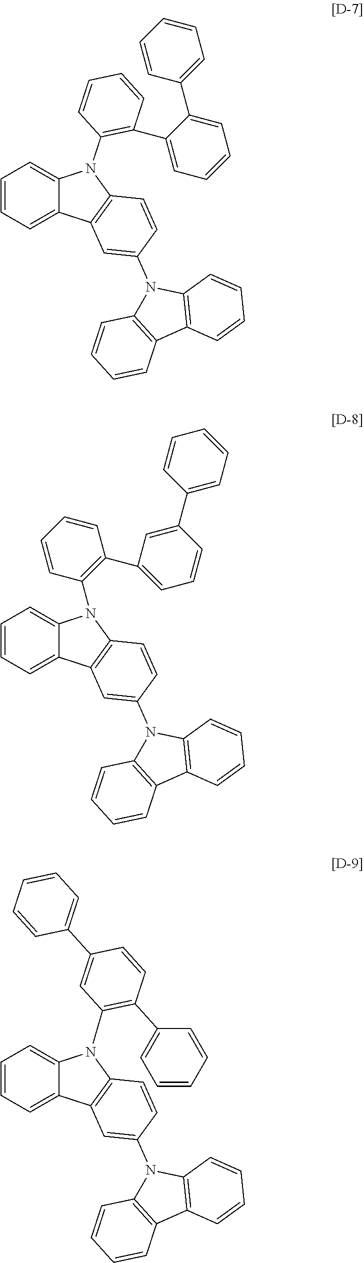 [SMILES: c1ccc(-c2ccccc2-c2ccccc2-n2c3ccccc3c3cc(-n4c5ccccc5c5ccccc54)ccc32)cc1 ; c1ccc(-c2cccc(-c3ccccc3-n3c4ccccc4c4cc(-n5c6ccccc6c6ccccc65)ccc43)c2)cc1 ; c1ccc(-c2ccc(-c3ccccc3)c(-n3c4ccccc4c4cc(-n5c6ccccc6c6ccccc65)ccc43)c2)cc1]

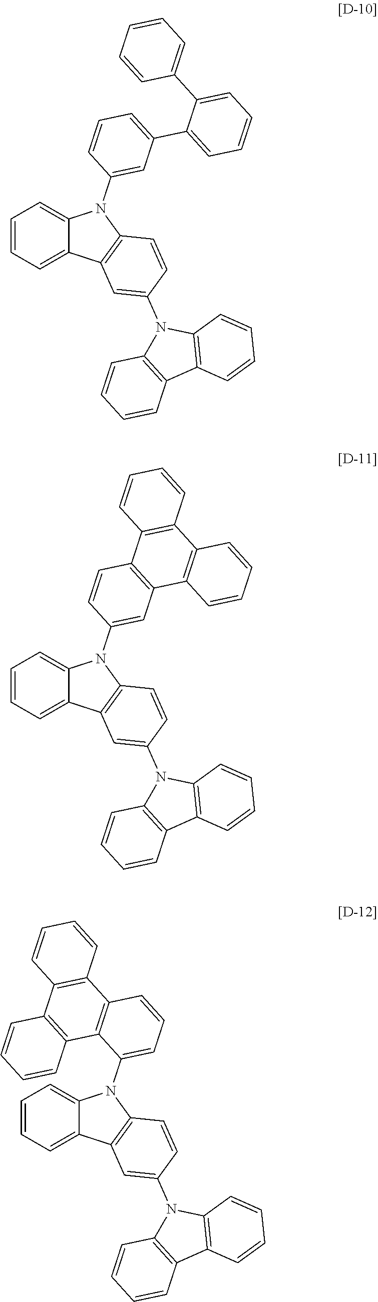 [SMILES: c1ccc(-c2ccccc2-c2cccc(-n3c4ccccc4c4cc(-n5c6ccccc6c6ccccc65)ccc43)c2)cc1 ; c1ccc2c(c1)c1ccccc1c1cc(-n3c4ccccc4c4cc(-n5c6ccccc6c6ccccc65)ccc43)ccc21 ; c1ccc2c(c1)c1ccccc1c1c(-n3c4ccccc4c4cc(-n5c6ccccc6c6ccccc65)ccc43)cccc21]

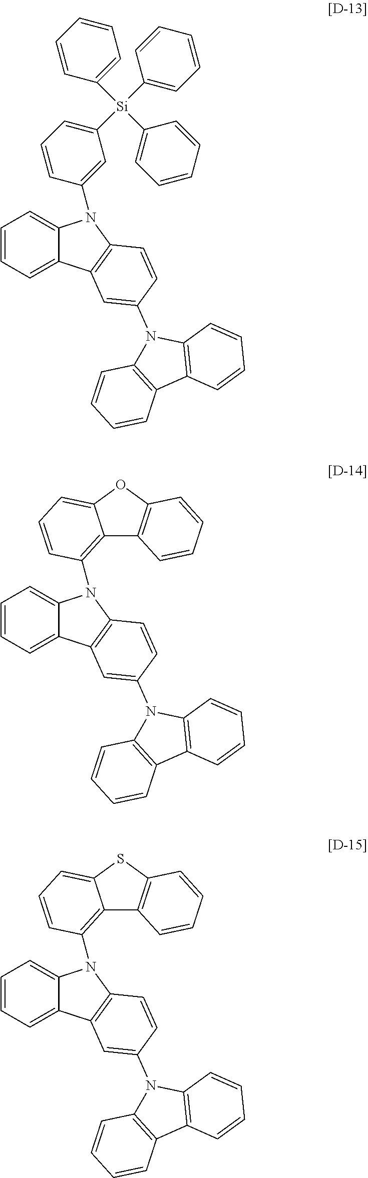 [SMILES: c1ccc([Si](c2ccccc2)(c2ccccc2)c2cccc(-n3c4ccccc4c4cc(-n5c6ccccc6c6ccccc65)ccc43)c2)cc1 ; c1ccc2c(c1)oc1cccc(-n3c4ccccc4c4cc(-n5c6ccccc6c6ccccc65)ccc43)c12 ; c1ccc2c(c1)sc1cccc(-n3c4ccccc4c4cc(-n5c6ccccc6c6ccccc65)ccc43)c12]

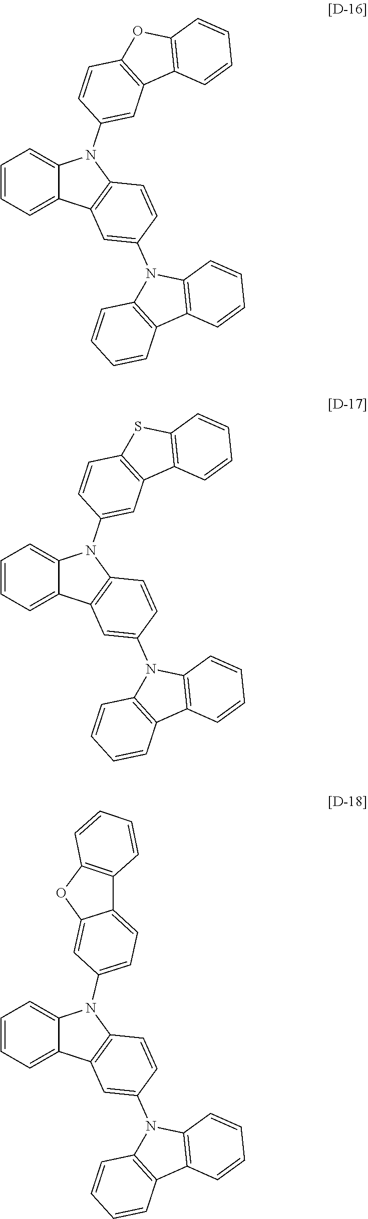 [SMILES: c1ccc2c(c1)oc1ccc(-n3c4ccccc4c4cc(-n5c6ccccc6c6ccccc65)ccc43)cc12 ; c1ccc2c(c1)sc1ccc(-n3c4ccccc4c4cc(-n5c6ccccc6c6ccccc65)ccc43)cc12 ; c1ccc2c(c1)oc1cc(-n3c4ccccc4c4cc(-n5c6ccccc6c6ccccc65)ccc43)ccc12]

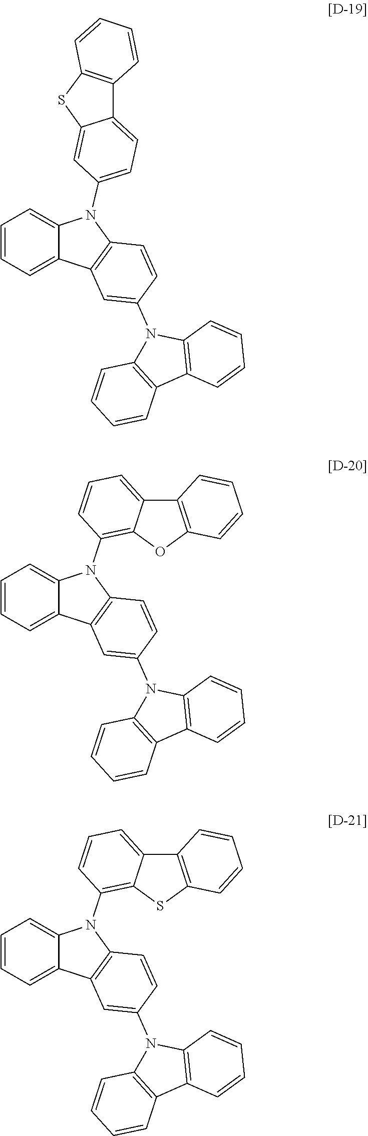 [SMILES: c1ccc2c(c1)sc1cc(-n3c4ccccc4c4cc(-n5c6ccccc6c6ccccc65)ccc43)ccc12 ; c1ccc2c(c1)oc1c(-n3c4ccccc4c4cc(-n5c6ccccc6c6ccccc65)ccc43)cccc12 ; c1ccc2c(c1)sc1c(-n3c4ccccc4c4cc(-n5c6ccccc6c6ccccc65)ccc43)cccc12]

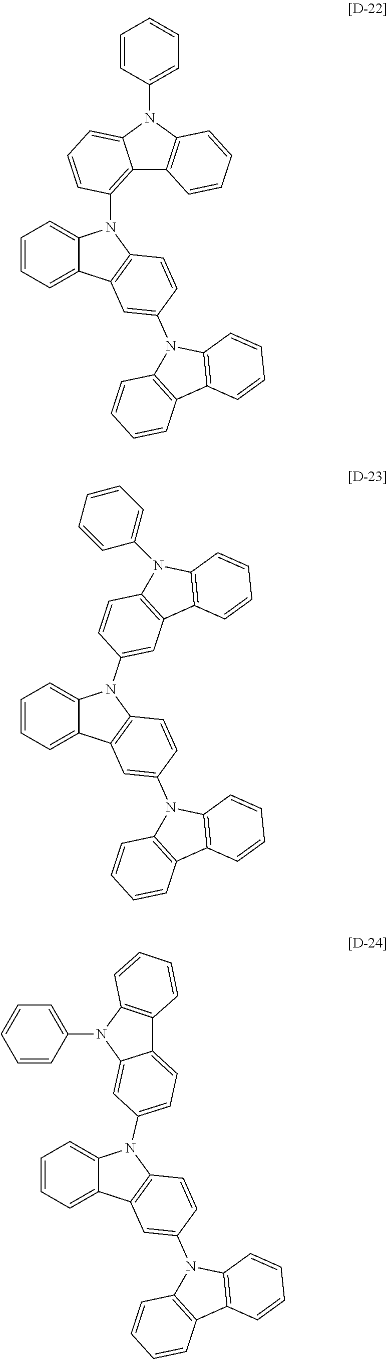 [SMILES: c1ccc(-n2c3ccccc3c3c(-n4c5ccccc5c5cc(-n6c7ccccc7c7ccccc76)ccc54)cccc32)cc1 ; c1ccc(-n2c3ccccc3c3cc(-n4c5ccccc5c5cc(-n6c7ccccc7c7ccccc76)ccc54)ccc32)cc1 ; c1ccc(-n2c3ccccc3c3ccc(-n4c5ccccc5c5cc(-n6c7ccccc7c7ccccc76)ccc54)cc32)cc1]

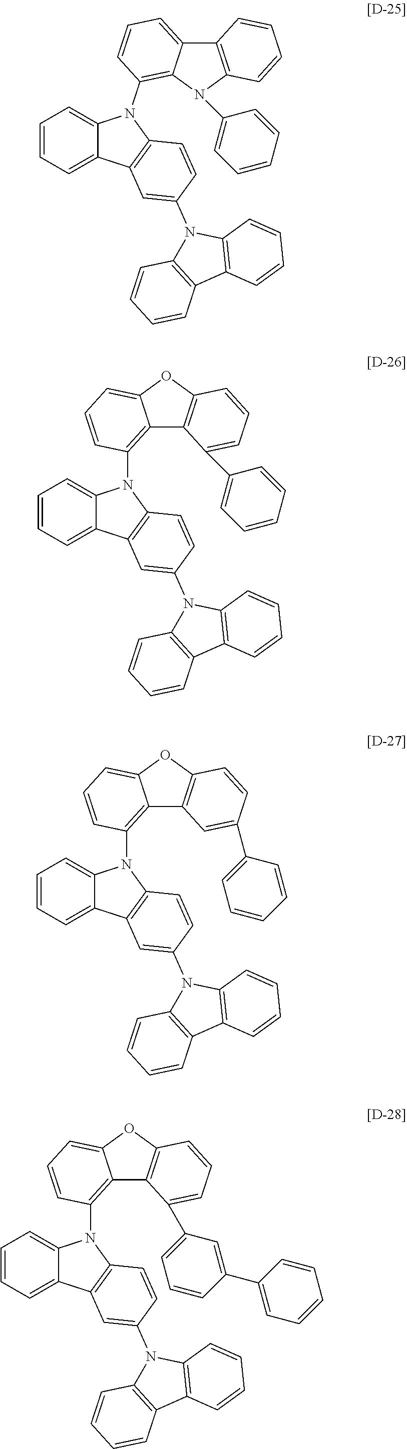 [SMILES: c1ccc(-n2c3ccccc3c3cccc(-n4c5ccccc5c5cc(-n6c7ccccc7c7ccccc76)ccc54)c32)cc1 ; c1ccc(-c2cccc3oc4cccc(-n5c6ccccc6c6cc(-n7c8ccccc8c8ccccc87)ccc65)c4c23)cc1 ; c1ccc(-c2ccc3oc4cccc(-n5c6ccccc6c6cc(-n7c8ccccc8c8ccccc87)ccc65)c4c3c2)cc1 ; c1ccc(-c2cccc(-c3cccc4oc5cccc(-n6c7ccccc7c7cc(-n8c9ccccc9c9ccccc98)ccc76)c5c34)c2)cc1]

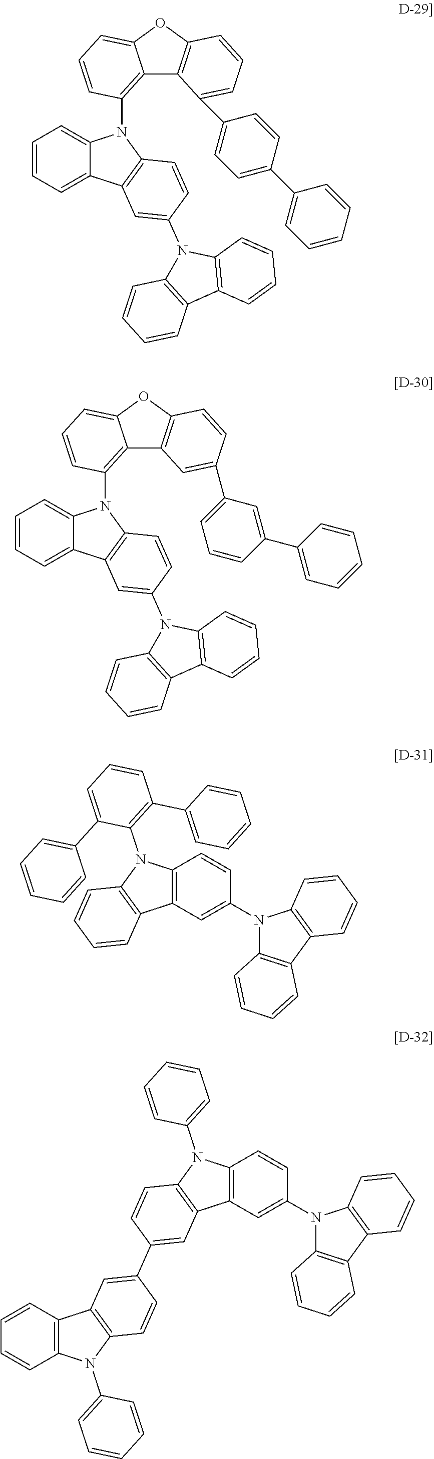 [SMILES: c1ccc(-c2ccc(-c3cccc4oc5cccc(-n6c7ccccc7c7cc(-n8c9ccccc9c9ccccc98)ccc76)c5c34)cc2)cc1 ; c1ccc(-c2cccc(-c3ccc4oc5cccc(-n6c7ccccc7c7cc(-n8c9ccccc9c9ccccc98)ccc76)c5c4c3)c2)cc1 ; c1ccc(-c2cccc(-c3ccccc3)c2-n2c3ccccc3c3cc(-n4c5ccccc5c5ccccc54)ccc32)cc1 ; c1ccc(-n2c3ccccc3c3cc(-c4ccc5c(c4)c4cc(-n6c7ccccc7c7ccccc76)ccc4n5-c4ccccc4)ccc32)cc1]

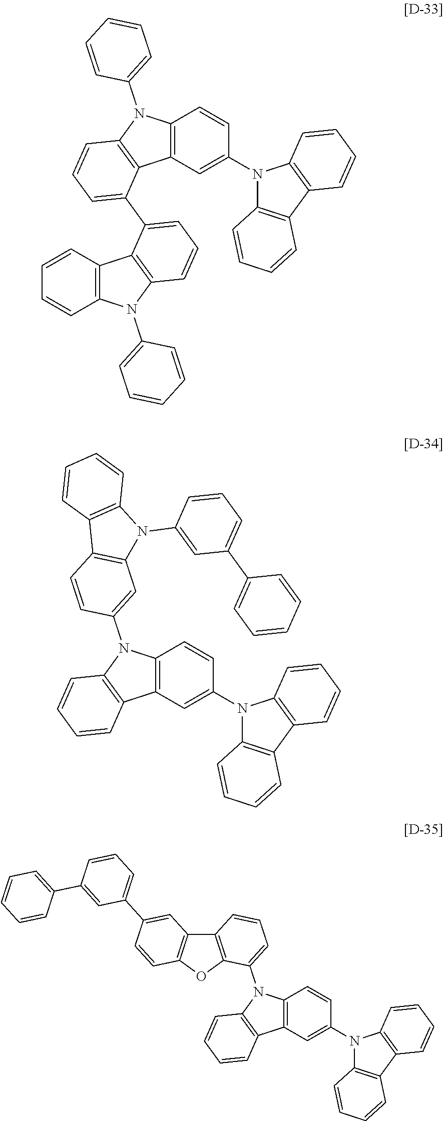 [SMILES: c1ccc(-n2c3ccccc3c3c(-c4cccc5c4c4cc(-n6c7ccccc7c7ccccc76)ccc4n5-c4ccccc4)cccc32)cc1 ; c1ccc(-c2cccc(-n3c4ccccc4c4ccc(-n5c6ccccc6c6cc(-n7c8ccccc8c8ccccc87)ccc65)cc43)c2)cc1 ; c1ccc(-c2cccc(-c3ccc4oc5c(-n6c7ccccc7c7cc(-n8c9ccccc9c9ccccc98)ccc76)cccc5c4c3)c2)cc1]

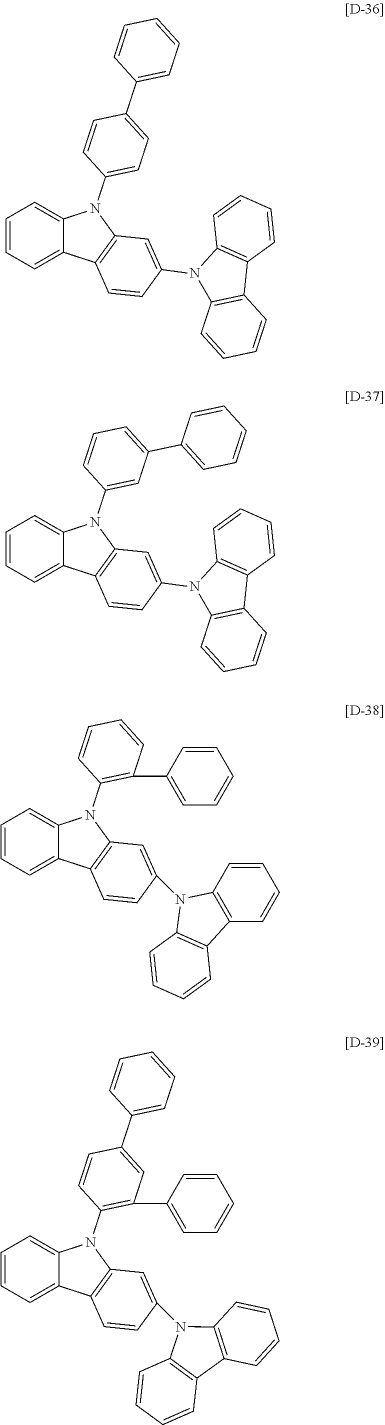 [SMILES: c1ccc(-c2ccc(-n3c4ccccc4c4ccc(-n5c6ccccc6c6ccccc65)cc43)cc2)cc1 ; c1ccc(-c2cccc(-n3c4ccccc4c4ccc(-n5c6ccccc6c6ccccc65)cc43)c2)cc1 ; c1ccc(-c2ccccc2-n2c3ccccc3c3ccc(-n4c5ccccc5c5ccccc54)cc32)cc1 ; c1ccc(-c2ccc(-n3c4ccccc4c4ccc(-n5c6ccccc6c6ccccc65)cc43)c(-c3ccccc3)c2)cc1]

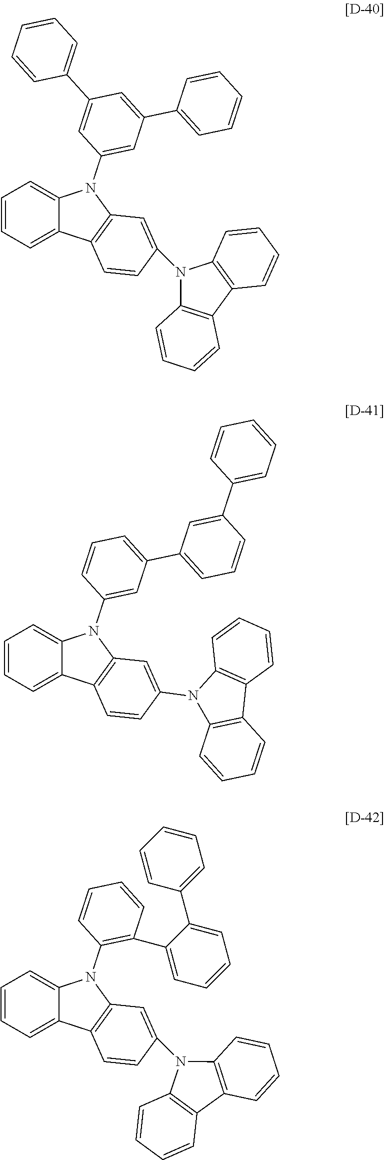 [SMILES: c1ccc(-c2cc(-c3ccccc3)cc(-n3c4ccccc4c4ccc(-n5c6ccccc6c6ccccc65)cc43)c2)cc1 ; c1ccc(-c2cccc(-c3cccc(-n4c5ccccc5c5ccc(-n6c7ccccc7c7ccccc76)cc54)c3)c2)cc1 ; c1ccc(-c2ccccc2-c2ccccc2-n2c3ccccc3c3ccc(-n4c5ccccc5c5ccccc54)cc32)cc1]

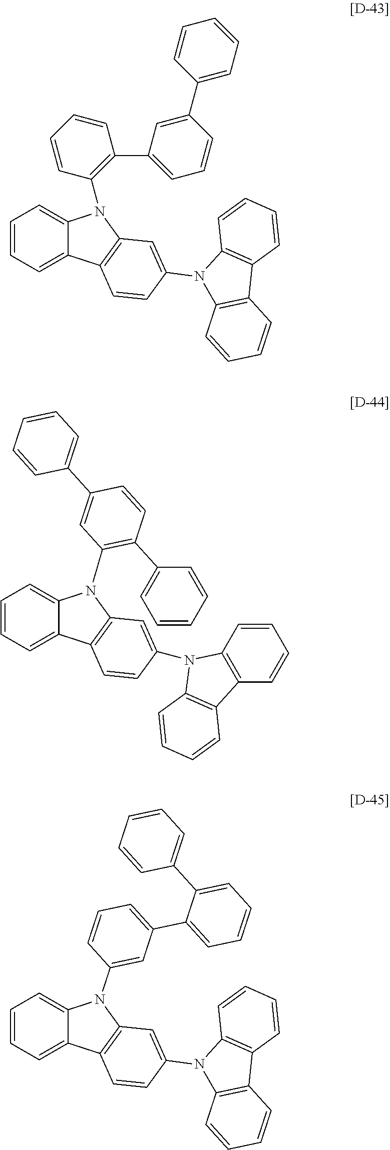 [SMILES: c1ccc(-c2cccc(-c3ccccc3-n3c4ccccc4c4ccc(-n5c6ccccc6c6ccccc65)cc43)c2)cc1 ; c1ccc(-c2ccc(-c3ccccc3)c(-n3c4ccccc4c4ccc(-n5c6ccccc6c6ccccc65)cc43)c2)cc1 ; c1ccc(-c2ccccc2-c2cccc(-n3c4ccccc4c4ccc(-n5c6ccccc6c6ccccc65)cc43)c2)cc1]

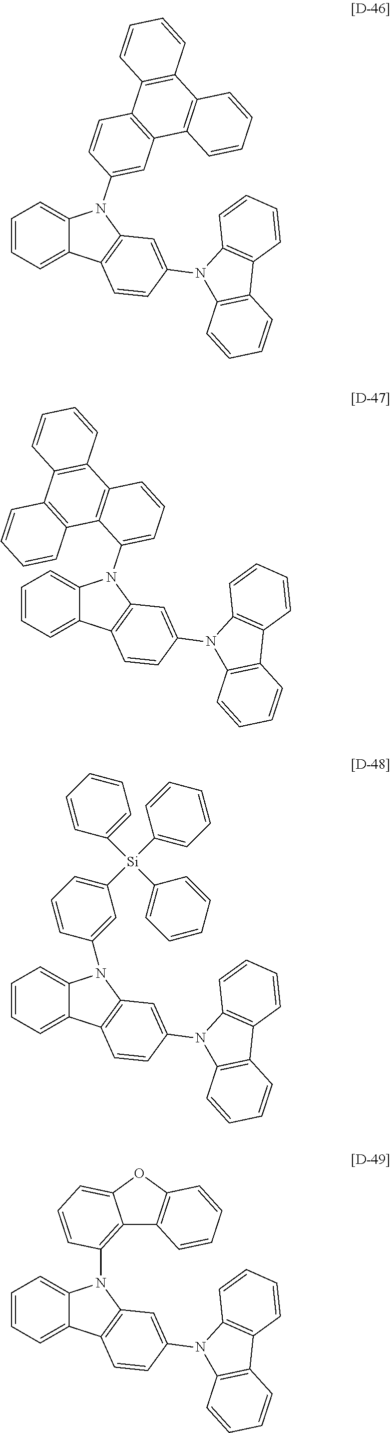 [SMILES: c1ccc2c(c1)c1ccccc1c1cc(-n3c4ccccc4c4ccc(-n5c6ccccc6c6ccccc65)cc43)ccc21 ; c1ccc2c(c1)c1ccccc1c1c(-n3c4ccccc4c4ccc(-n5c6ccccc6c6ccccc65)cc43)cccc21 ; c1ccc([Si](c2ccccc2)(c2ccccc2)c2cccc(-n3c4ccccc4c4ccc(-n5c6ccccc6c6ccccc65)cc43)c2)cc1 ; c1ccc2c(c1)oc1cccc(-n3c4ccccc4c4ccc(-n5c6ccccc6c6ccccc65)cc43)c12]

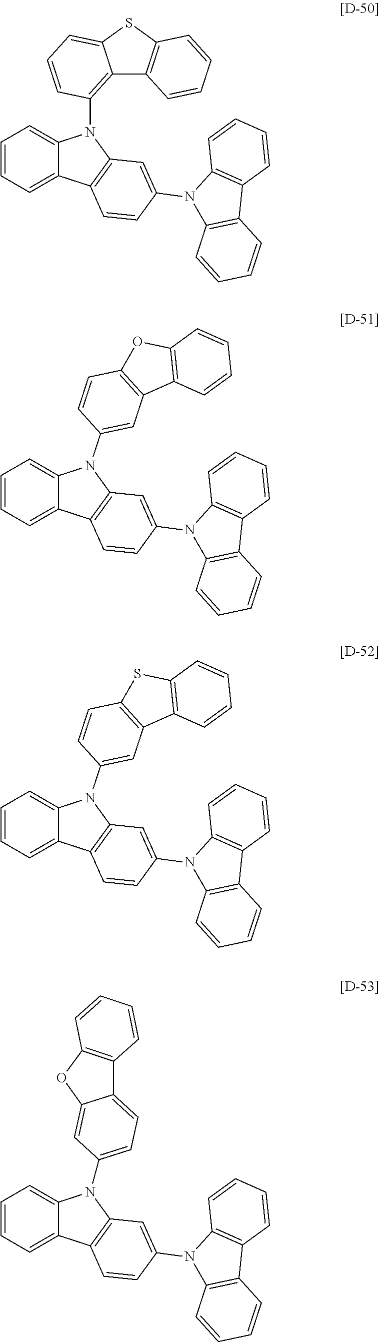 [SMILES: c1ccc2c(c1)sc1cccc(-n3c4ccccc4c4ccc(-n5c6ccccc6c6ccccc65)cc43)c12 ; c1ccc2c(c1)oc1ccc(-n3c4ccccc4c4ccc(-n5c6ccccc6c6ccccc65)cc43)cc12 ; c1ccc2c(c1)sc1ccc(-n3c4ccccc4c4ccc(-n5c6ccccc6c6ccccc65)cc43)cc12 ; c1ccc2c(c1)oc1cc(-n3c4ccccc4c4ccc(-n5c6ccccc6c6ccccc65)cc43)ccc12]

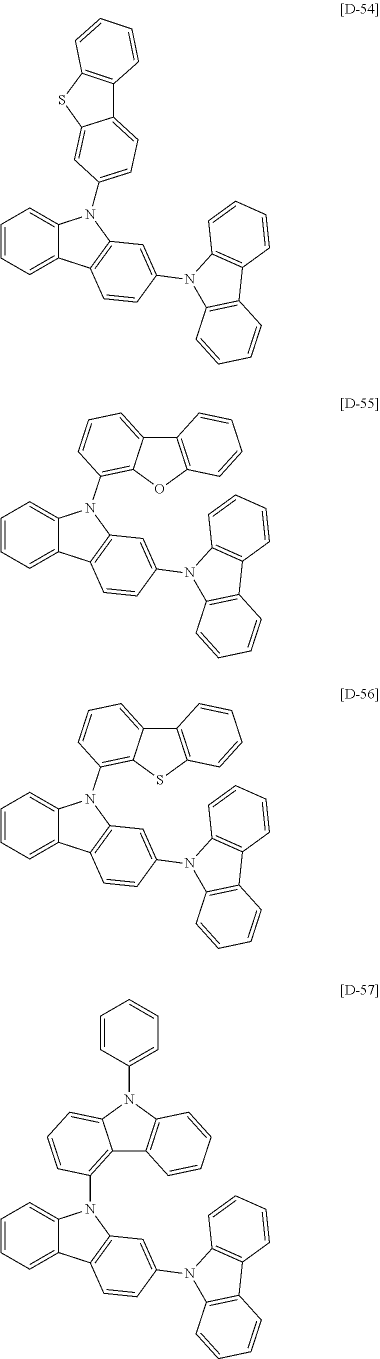 [SMILES: c1ccc2c(c1)sc1cc(-n3c4ccccc4c4ccc(-n5c6ccccc6c6ccccc65)cc43)ccc12 ; c1ccc2c(c1)oc1c(-n3c4ccccc4c4ccc(-n5c6ccccc6c6ccccc65)cc43)cccc12 ; c1ccc2c(c1)sc1c(-n3c4ccccc4c4ccc(-n5c6ccccc6c6ccccc65)cc43)cccc12 ; c1ccc(-n2c3ccccc3c3c(-n4c5ccccc5c5ccc(-n6c7ccccc7c7ccccc76)cc54)cccc32)cc1]

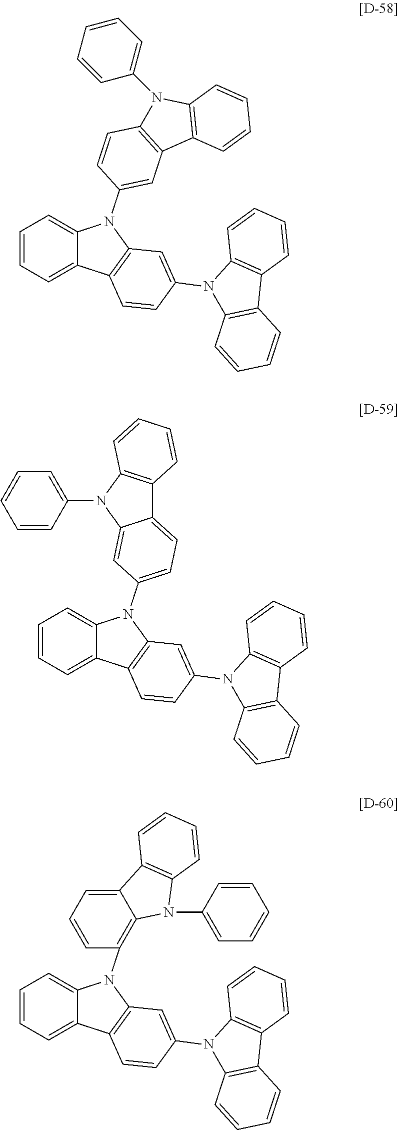 [SMILES: c1ccc(-n2c3ccccc3c3cc(-n4c5ccccc5c5ccc(-n6c7ccccc7c7ccccc76)cc54)ccc32)cc1 ; c1ccc(-n2c3ccccc3c3ccc(-n4c5ccccc5c5ccc(-n6c7ccccc7c7ccccc76)cc54)cc32)cc1 ; c1ccc(-n2c3ccccc3c3cccc(-n4c5ccccc5c5ccc(-n6c7ccccc7c7ccccc76)cc54)c32)cc1]

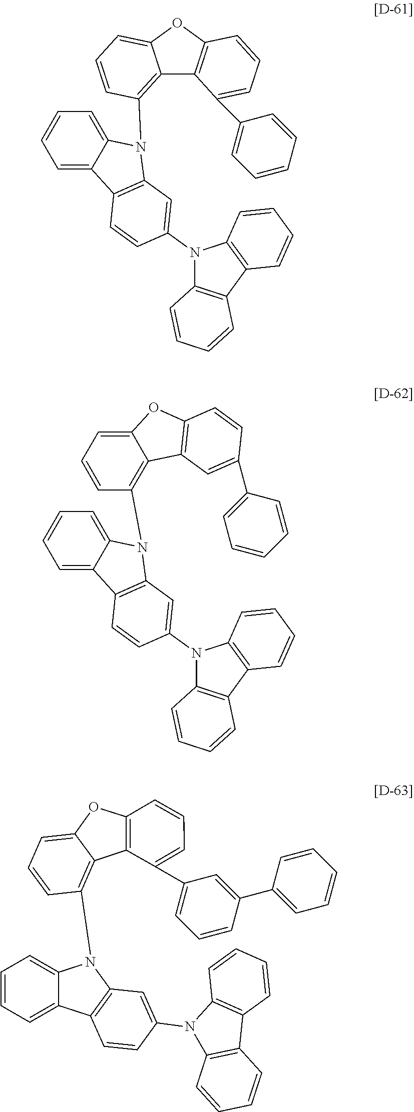 [SMILES: c1ccc(-c2cccc3oc4cccc(-n5c6ccccc6c6ccc(-n7c8ccccc8c8ccccc87)cc65)c4c23)cc1 ; c1ccc(-c2ccc3oc4cccc(-n5c6ccccc6c6ccc(-n7c8ccccc8c8ccccc87)cc65)c4c3c2)cc1 ; c1ccc(-c2cccc(-c3cccc4oc5cccc(-n6c7ccccc7c7ccc(-n8c9ccccc9c9ccccc98)cc76)c5c34)c2)cc1]

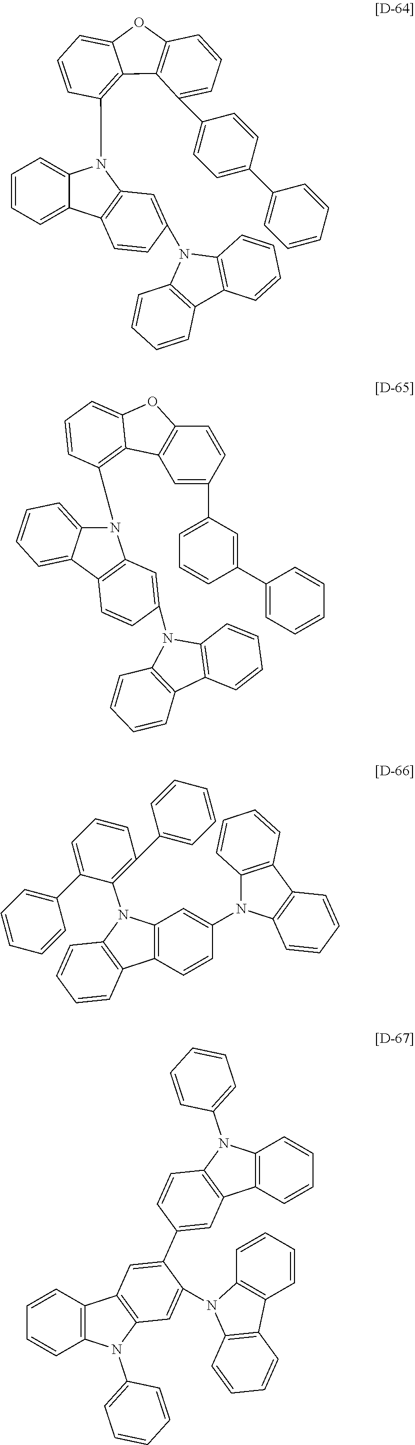 [SMILES: c1ccc(-c2ccc(-c3cccc4oc5cccc(-n6c7ccccc7c7ccc(-n8c9ccccc9c9ccccc98)cc76)c5c34)cc2)cc1 ; c1ccc(-c2cccc(-c3ccc4oc5cccc(-n6c7ccccc7c7ccc(-n8c9ccccc9c9ccccc98)cc76)c5c4c3)c2)cc1 ; c1ccc(-c2cccc(-c3ccccc3)c2-n2c3ccccc3c3ccc(-n4c5ccccc5c5ccccc54)cc32)cc1 ; c1ccc(-n2c3ccccc3c3cc(-c4cc5c6ccccc6n(-c6ccccc6)c5cc4-n4c5ccccc5c5ccccc54)ccc32)cc1]

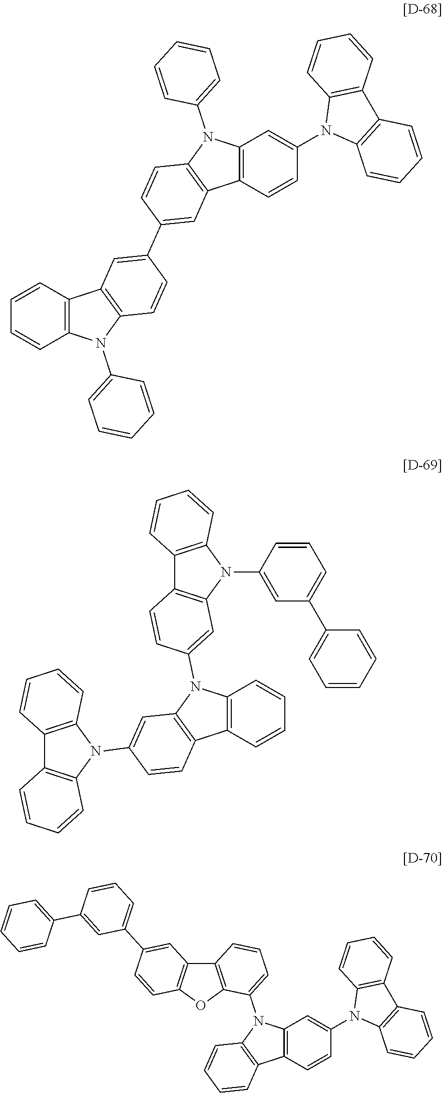 [SMILES: c1ccc(-n2c3ccccc3c3cc(-c4ccc5c(c4)c4ccc(-n6c7ccccc7c7ccccc76)cc4n5-c4ccccc4)ccc32)cc1 ; c1ccc(-c2cccc(-n3c4ccccc4c4ccc(-n5c6ccccc6c6ccc(-n7c8ccccc8c8ccccc87)cc65)cc43)c2)cc1 ; c1ccc(-c2cccc(-c3ccc4oc5c(-n6c7ccccc7c7ccc(-n8c9ccccc9c9ccccc98)cc76)cccc5c4c3)c2)cc1]

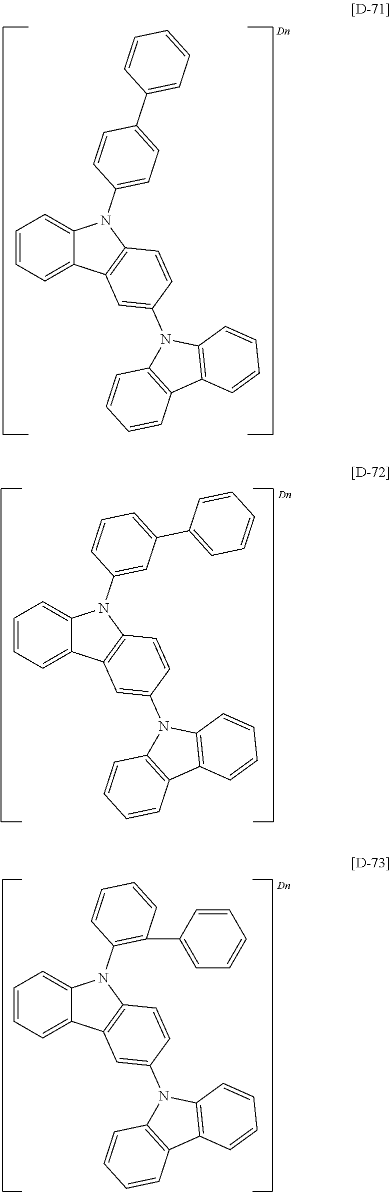

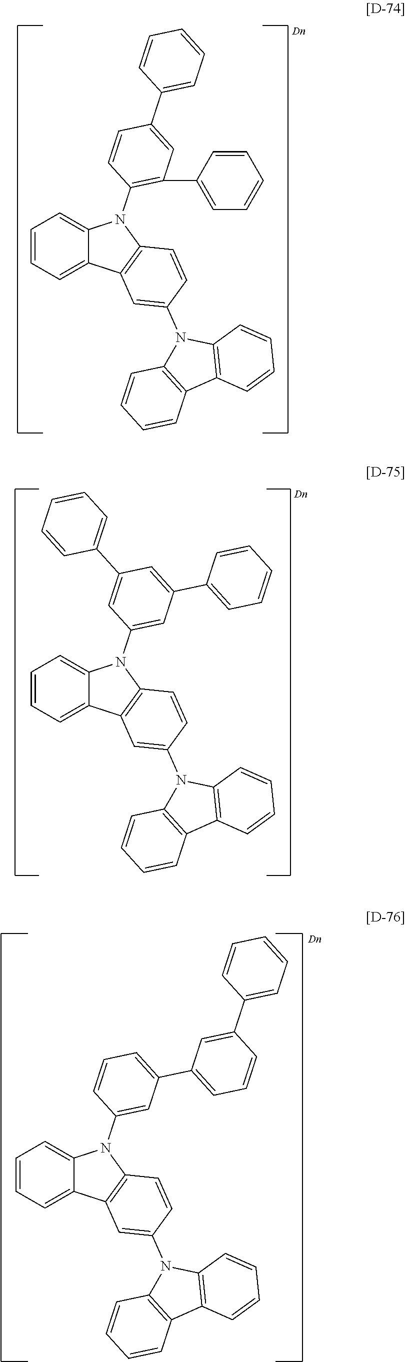

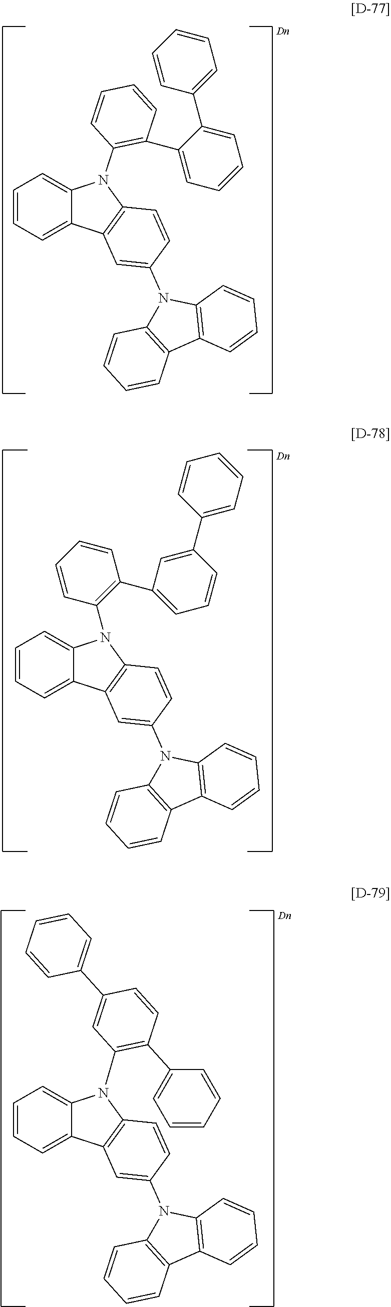

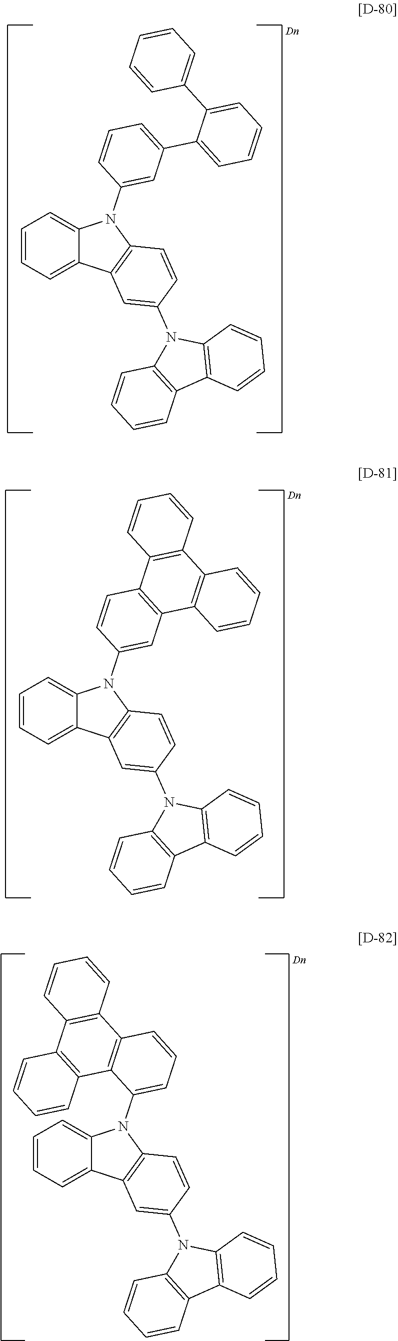

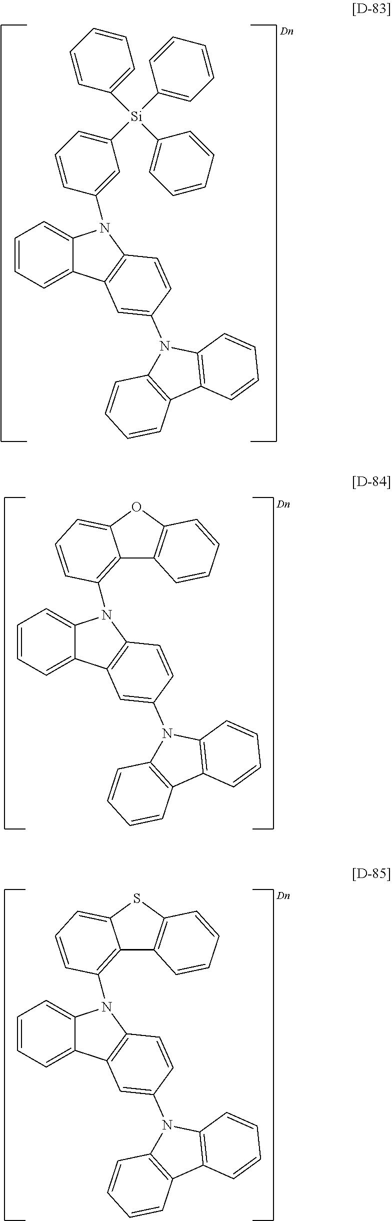

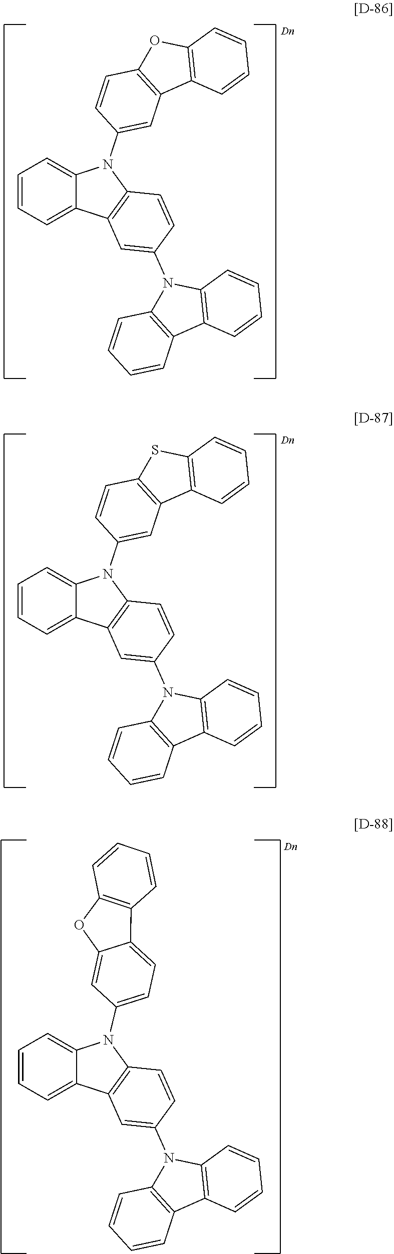

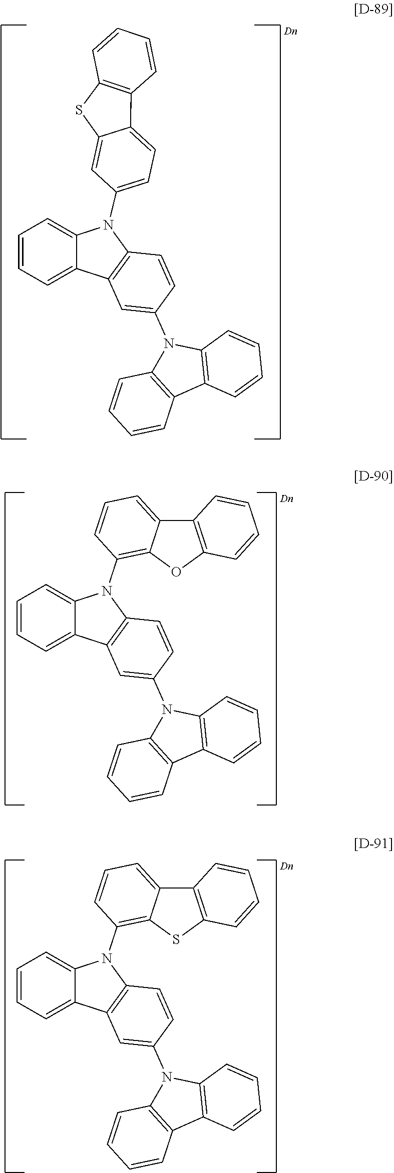

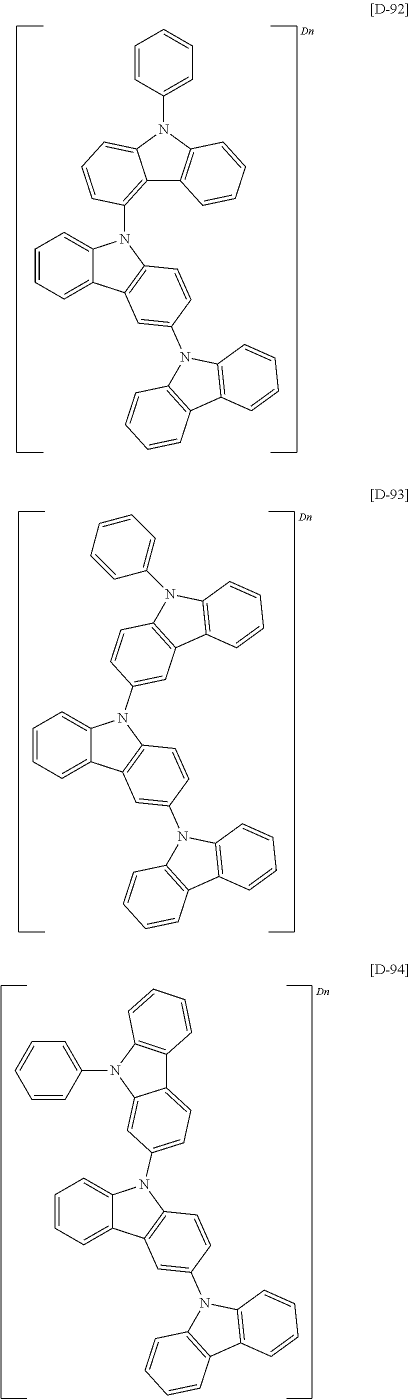

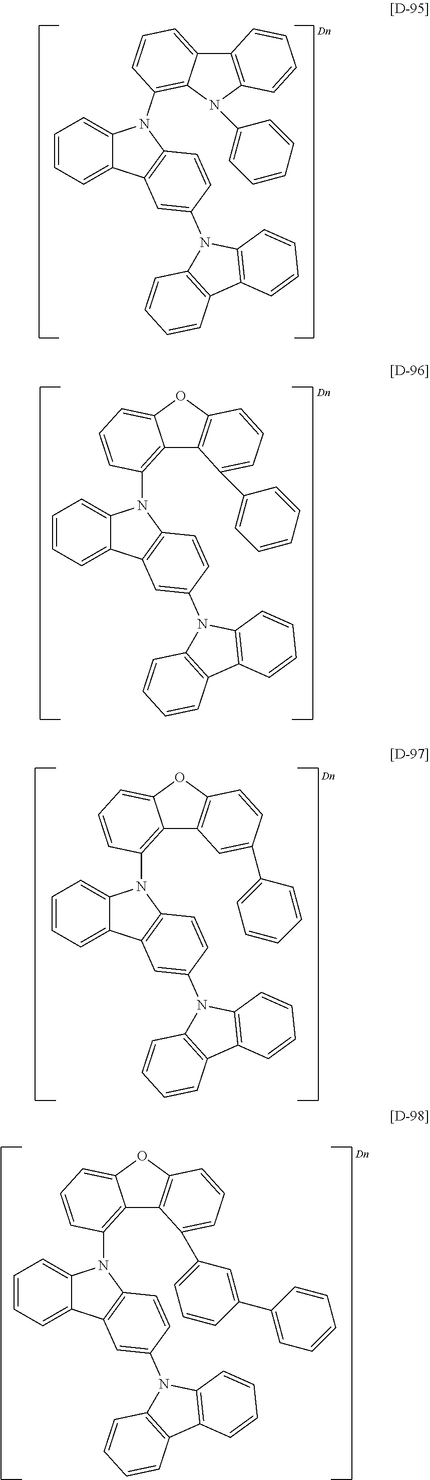

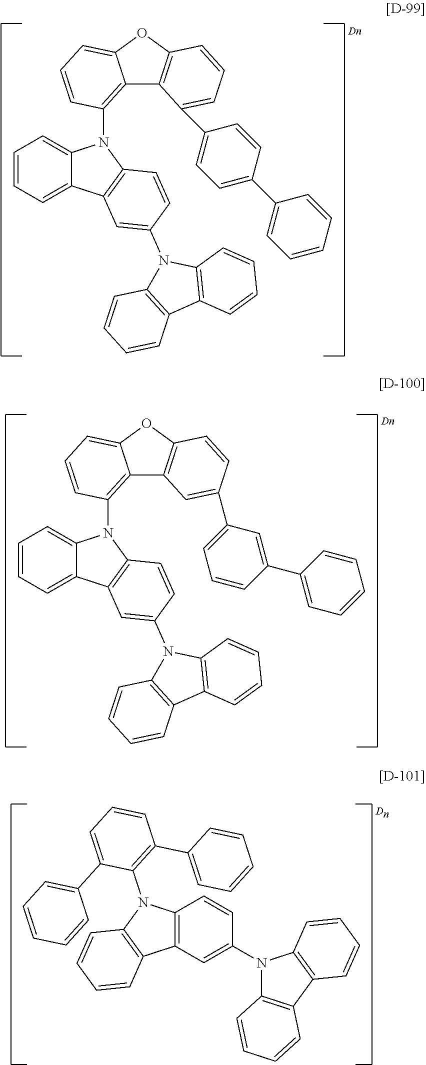

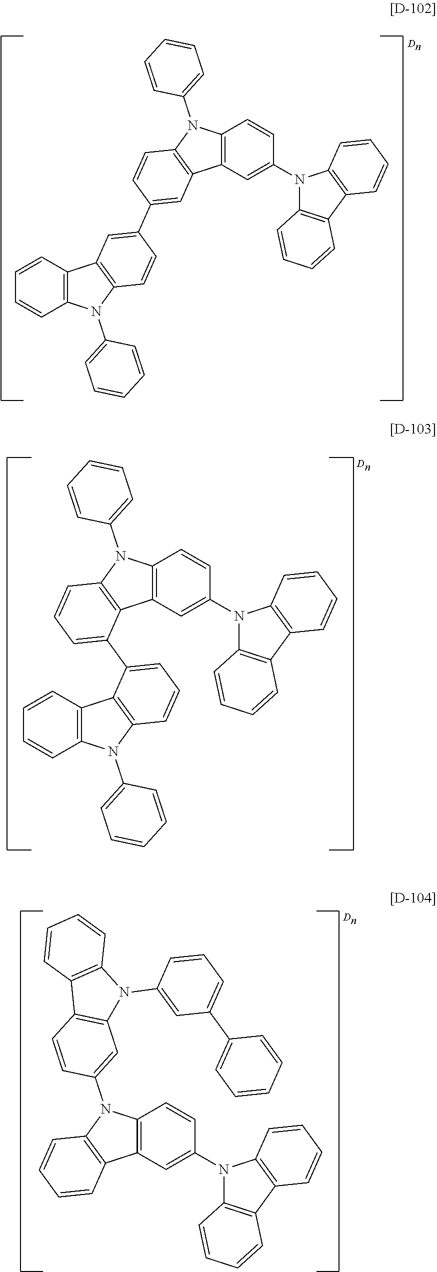

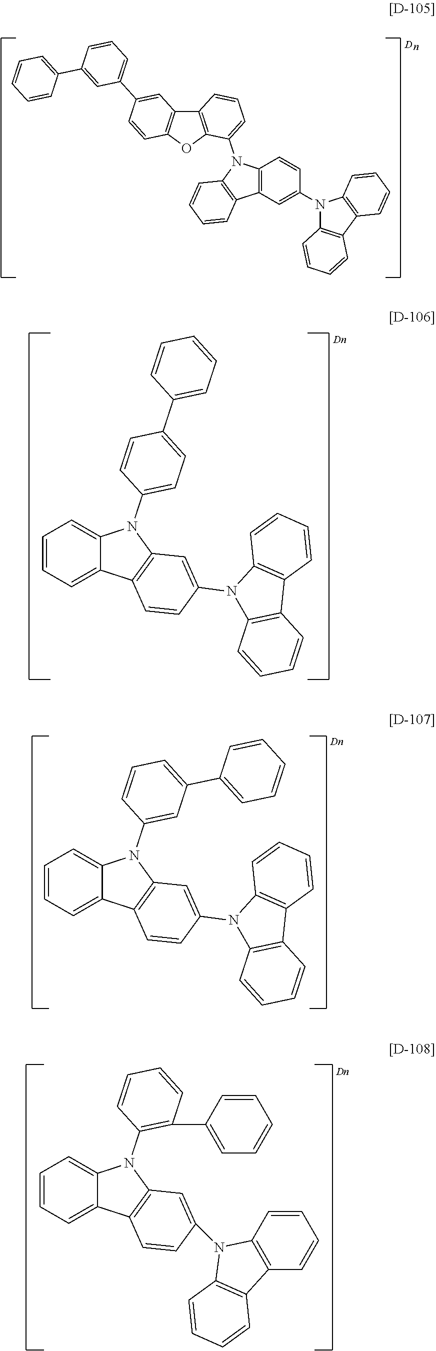

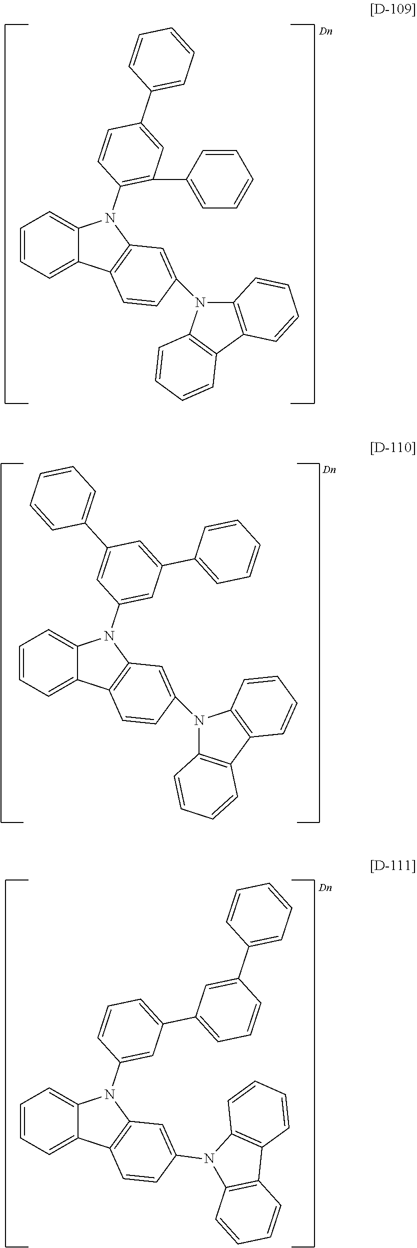

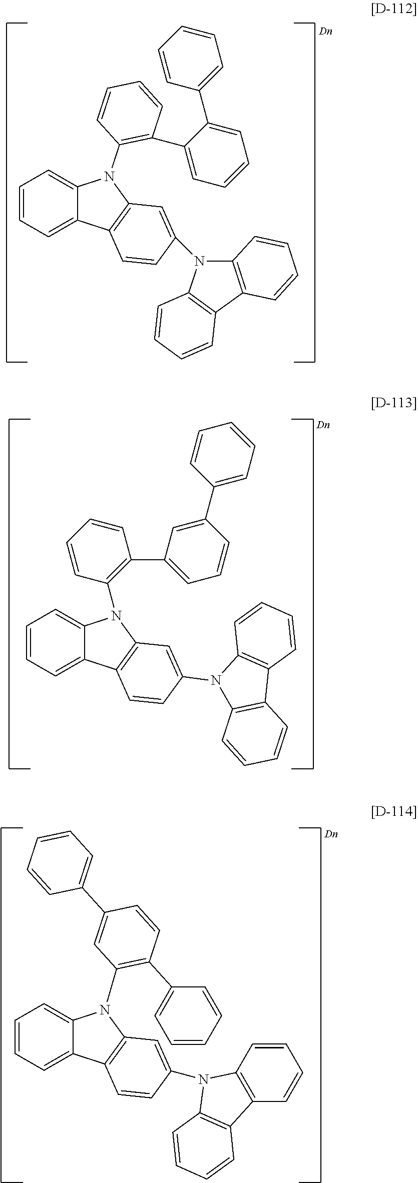

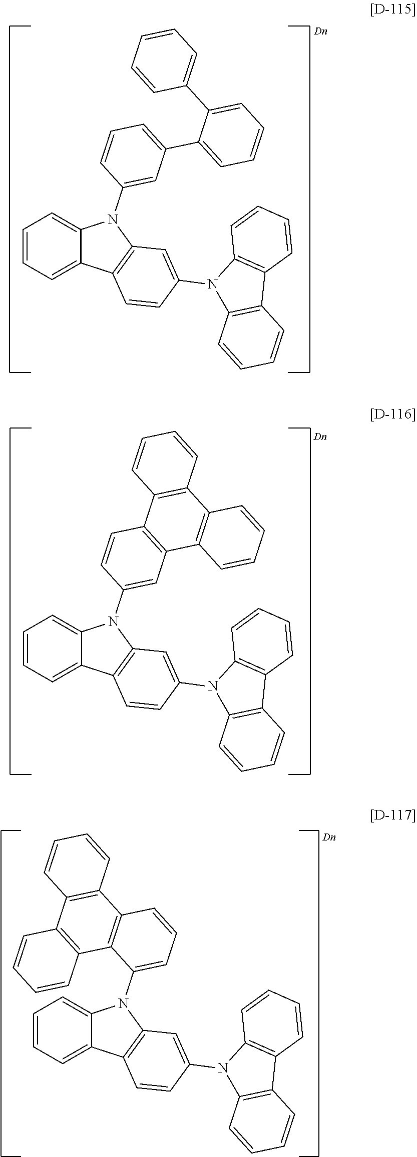

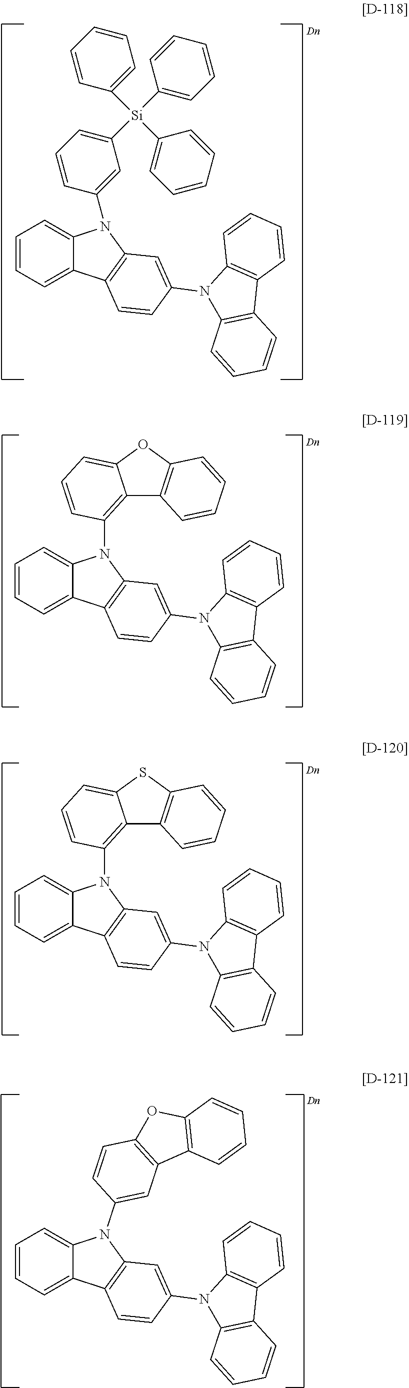

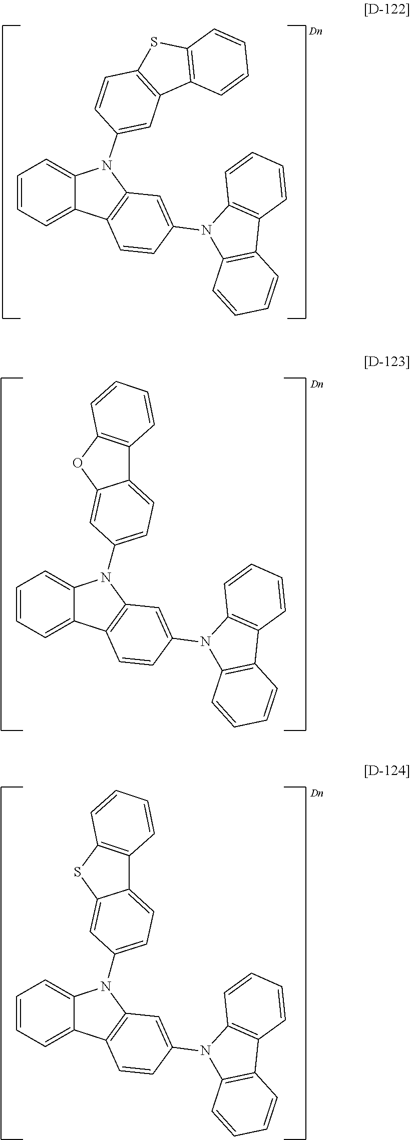

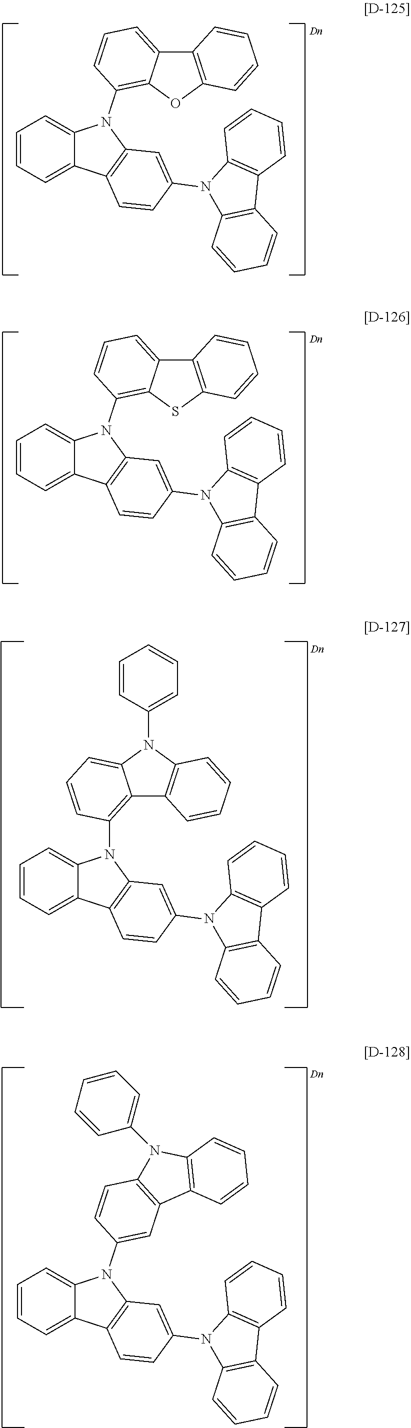

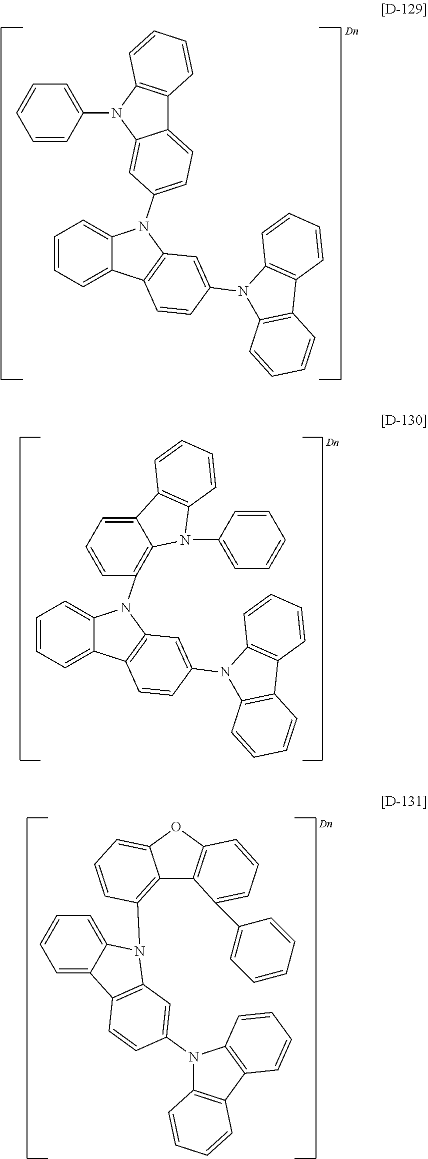

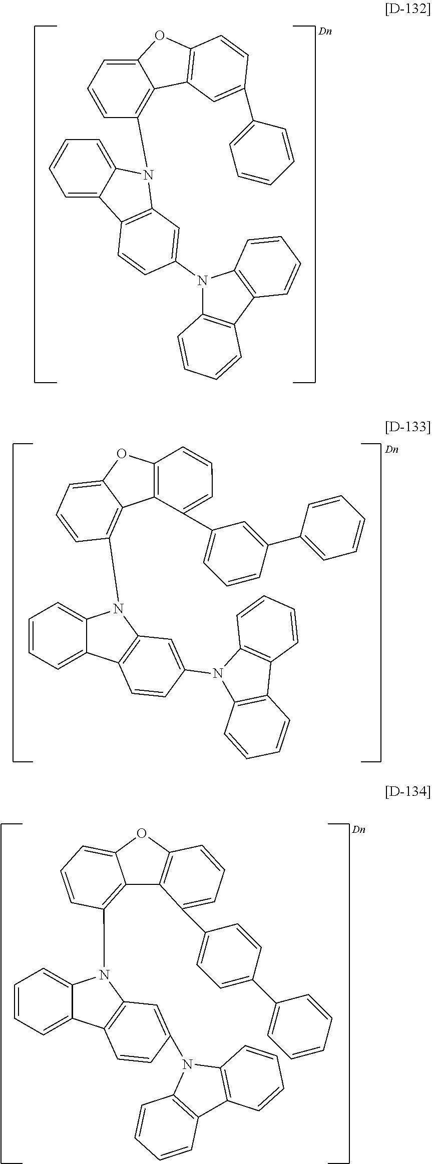

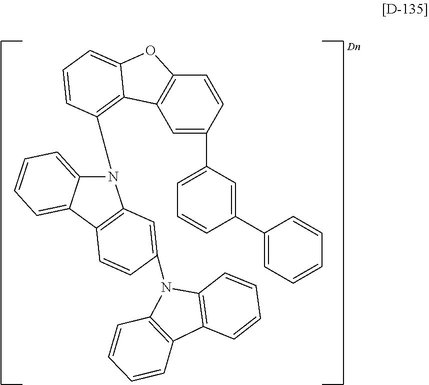

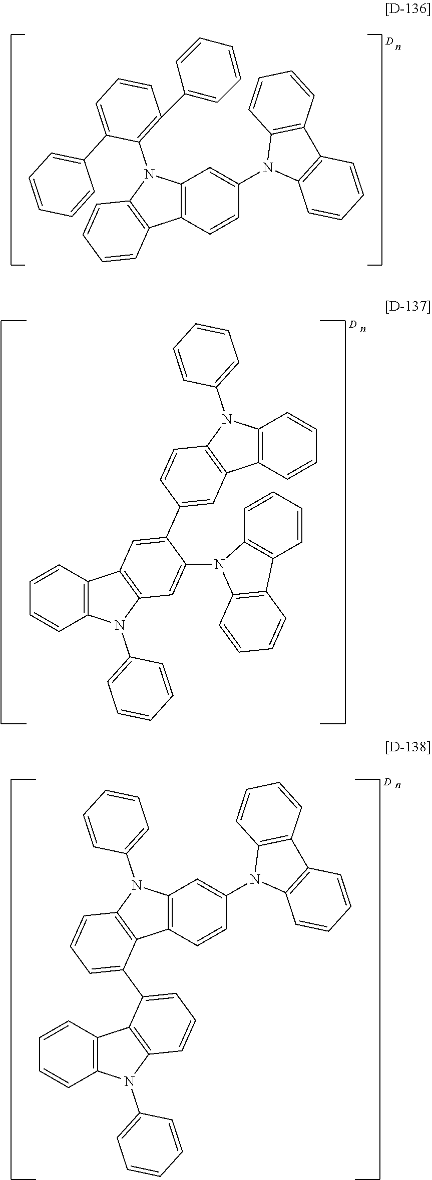

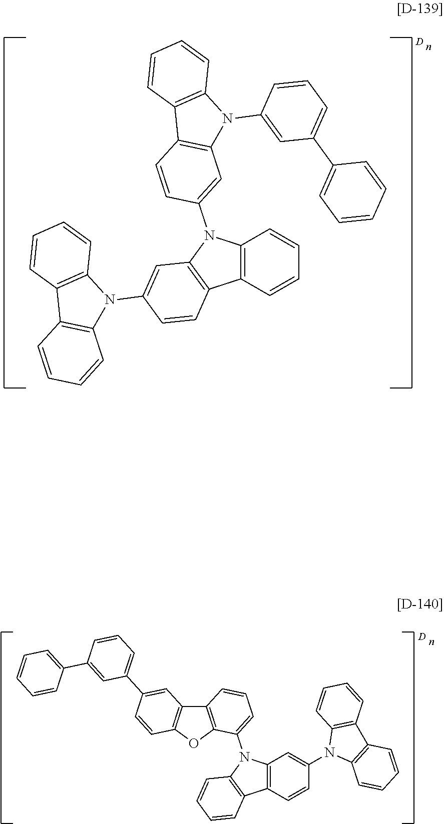

(Dn indicates the number of hydrogen atoms replaced by deuterium, and indicates a structure in which one or more deuterium atoms are substituted.) However, as noted above, any hydrogen in any compound may be protium, deuterium, or tritium, based on natural or artificial substitution.

The composition for an organic optoelectronic device may include the first compound and the second compound in various ratios.

In an implementation, the composition for an organic optoelectronic device may include the first compound and the second compound in a weight ratio of about 10:90 to about 90:10, e.g., in a weight ratio of about 20:80 to about 80:20, about 30:70 to about 70:30, about 40:60 to about 60:40, or about 50:50.

In an implementation, the first compound may be included in an amount equal to or greater than the second compound. In an implementation, the first compound may be included in about 50 wt % to about 90 wt %, based on a total amount of the first compound and the second compound.

In an implementation, the first compound may be included in less or the same amount as the second compound. In an implementation, the first compound may be included in about 10 wt % to about 50 wt %, based on a total amount of the first compound and the second compound.

The composition for an organic optoelectronic device may further include, e.g., a light-emitting dopant in addition to the first compound and the second compound.

The light-emitting dopant is a material mixed with the composition for an organic optoelectronic device in a small amount to cause light emission, and may be generally a material, e.g., a metal complex that emits light by multiple excitation into a triplet or more. The light-emitting dopant may be, e.g., an inorganic, organic, or organic/inorganic compound, and may be included in one or two or more types.

The light-emitting dopant may be, e.g., a phosphorescent sensitizer, a fluorescent dopant, or a combination thereof.

The phosphorescent sensitizer may be an organometallic compound and may effectively transfer energy received from the host to the fluorescent dopant. The phosphorescent sensitizer may increase energy transfer to the fluorescent dopant, causing excitons formed in the light emitting layer to emit light quickly inside the light emitting layer, thereby reducing deterioration of the light emitting diode.

The phosphorescent sensitizer may be, e.g., an organo-metal compound including iridium (Ir), platinum (Pt), osmium (Os), titanium (Ti), zirconium (Zr), hafnium (Hf), europium (Eu), terbium (Tb), thulium (Tm), rhodium (Rh), or a combination thereof, and may be, e.g., an organo-metal compound including an organic ligand including a nitrogen-containing ring. The nitrogen-containing ring may be, e.g., substituted or unsubstituted pyridine, a substituted or unsubstituted pyrimidine, a substituted or unsubstituted triazine, a substituted or unsubstituted carbazole, a substituted or unsubstituted imidazole, a substituted or unsubstituted benzoimidazole, or a combination thereof.

The phosphorescent sensitizer may be, e.g., one of Compounds P1 to P52.

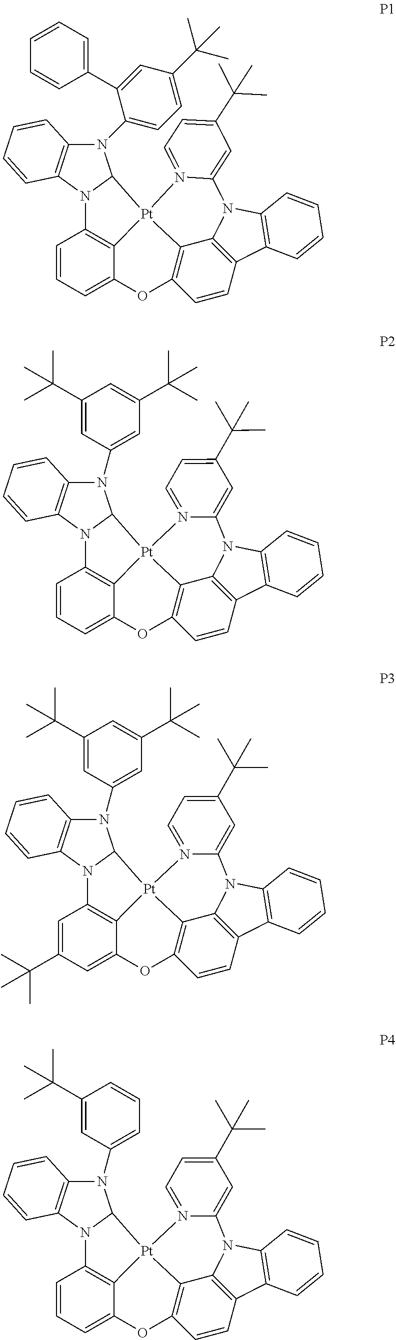

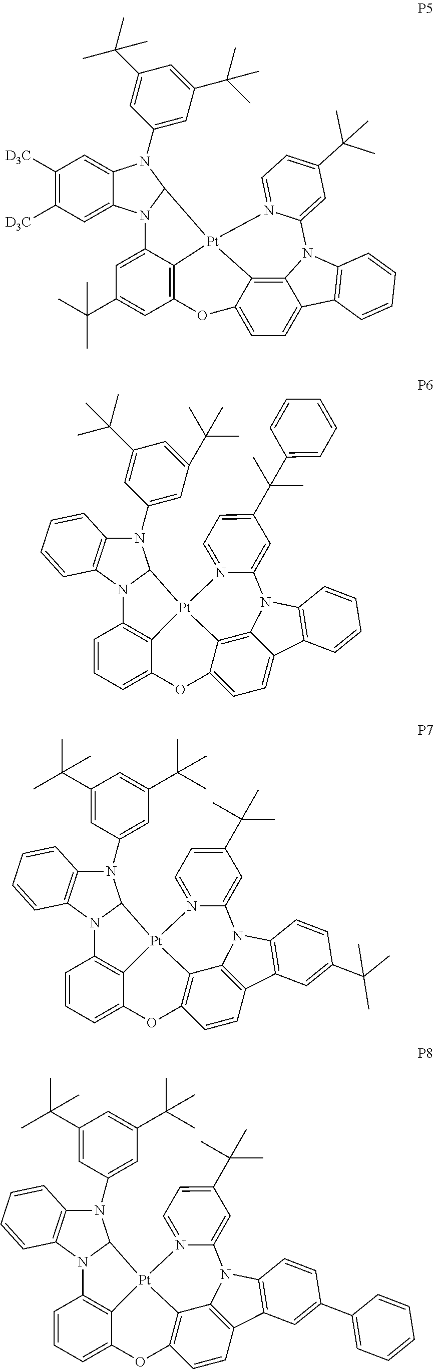

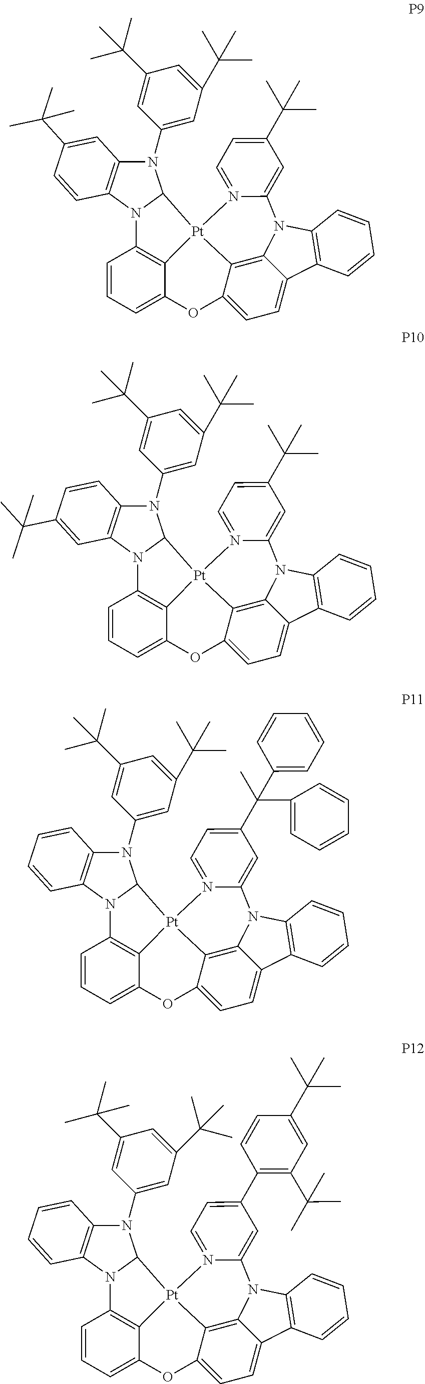

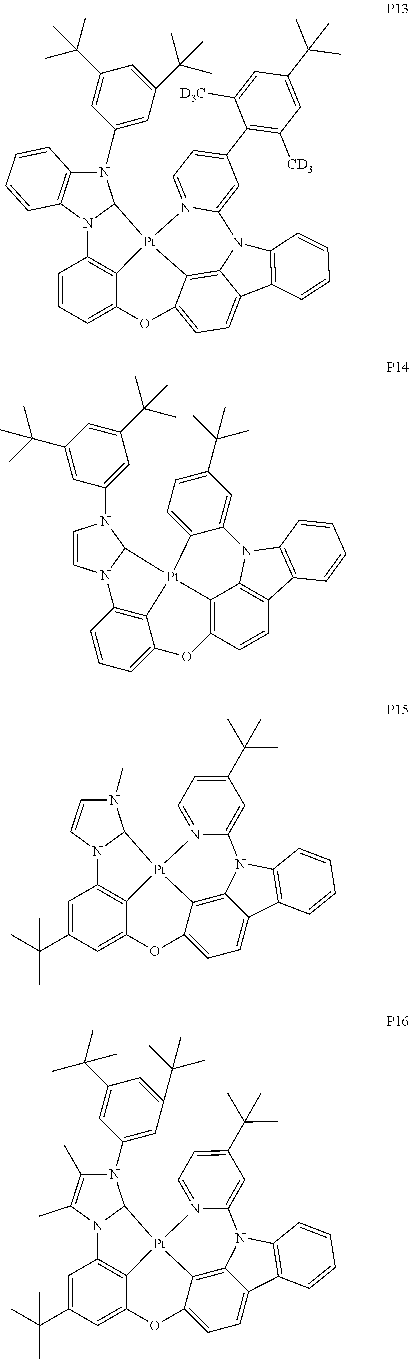

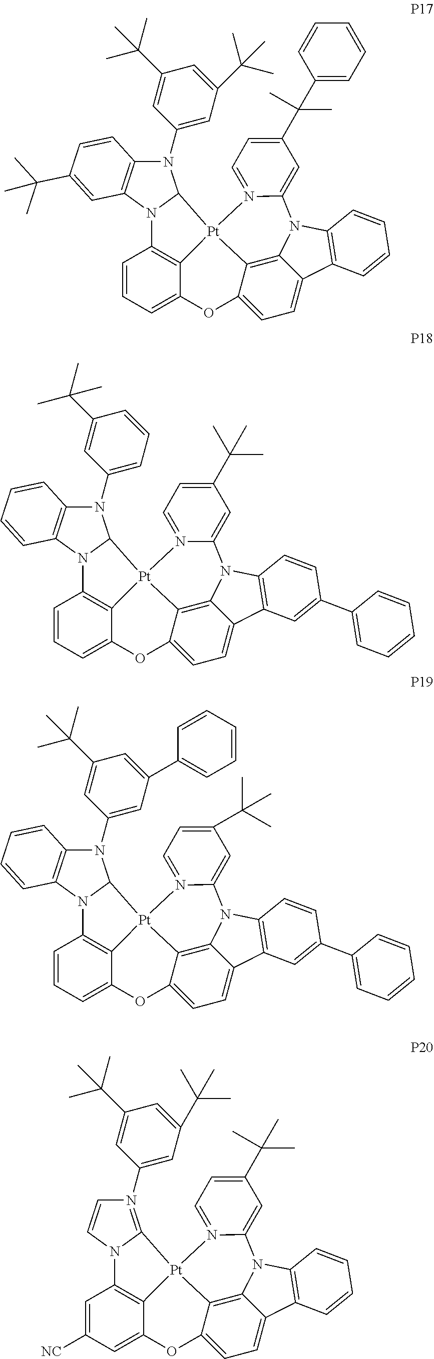

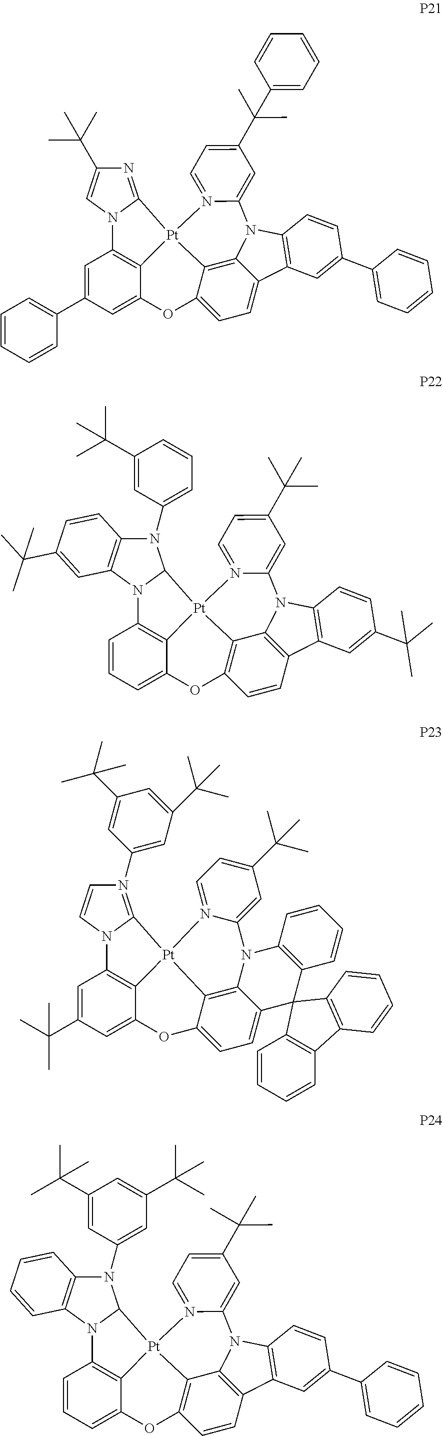

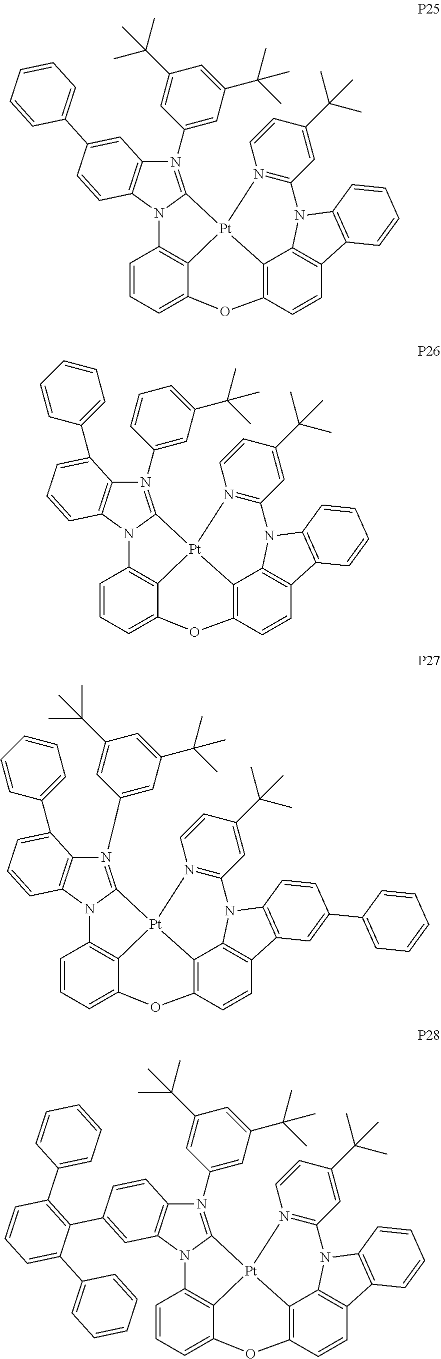

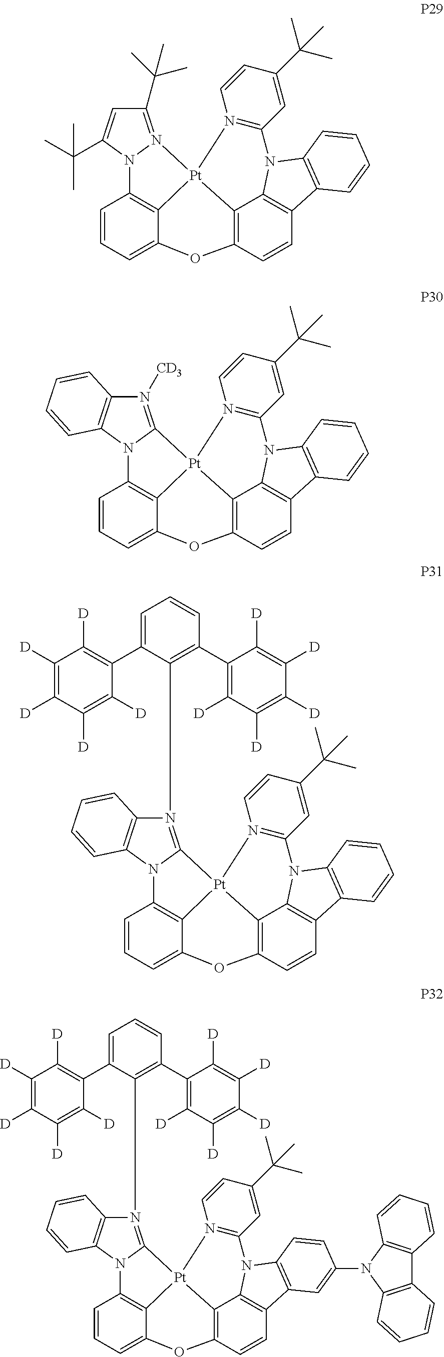

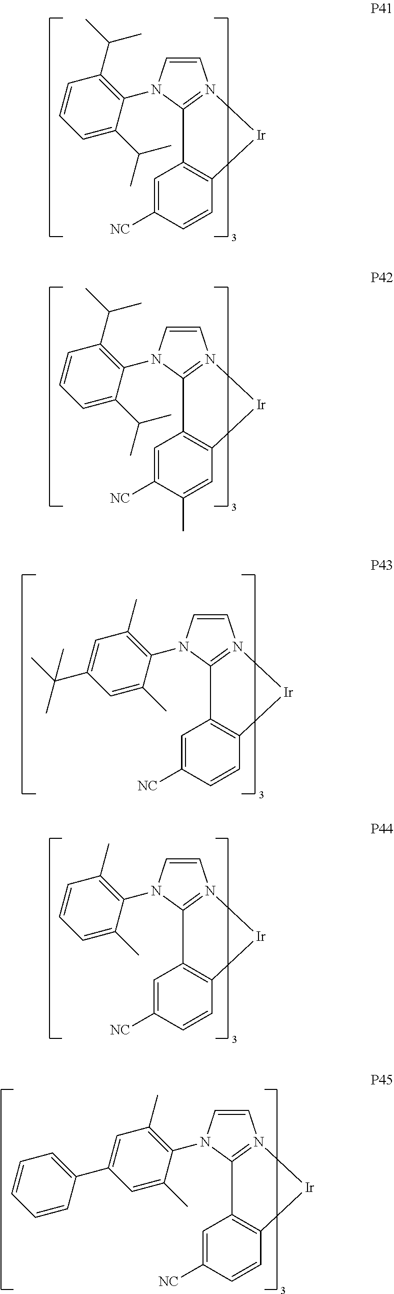

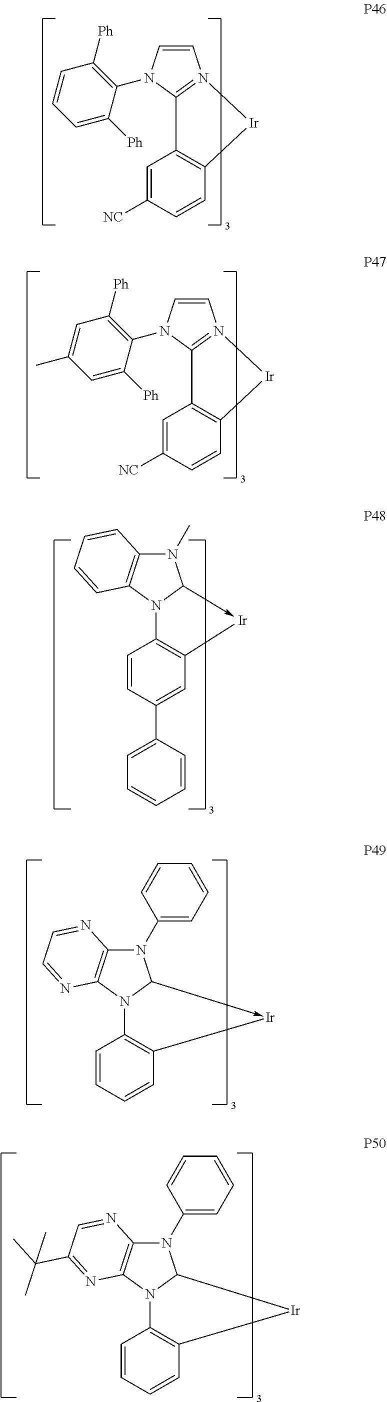

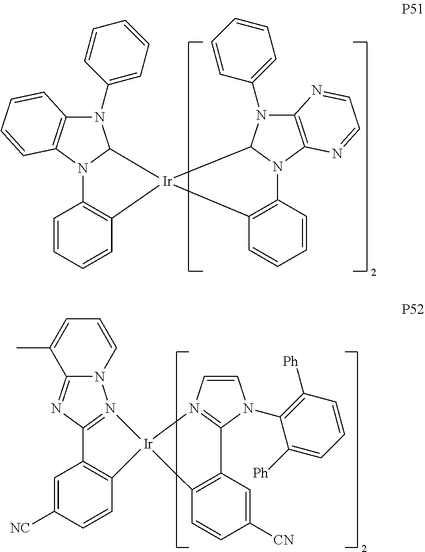

The fluorescent dopant may be, e.g., a polycyclic compound, and may improve the luminous efficiency and life-span characteristic of the light emitting diode by receiving energy transfer within the light emitting layer due to high absorbance.

The fluorescent dopant may be, e.g., a condensed polycyclic compound including boron (B), nitrogen (N), or a combination thereof, and may be, e.g., one of Compounds D1 to D30.

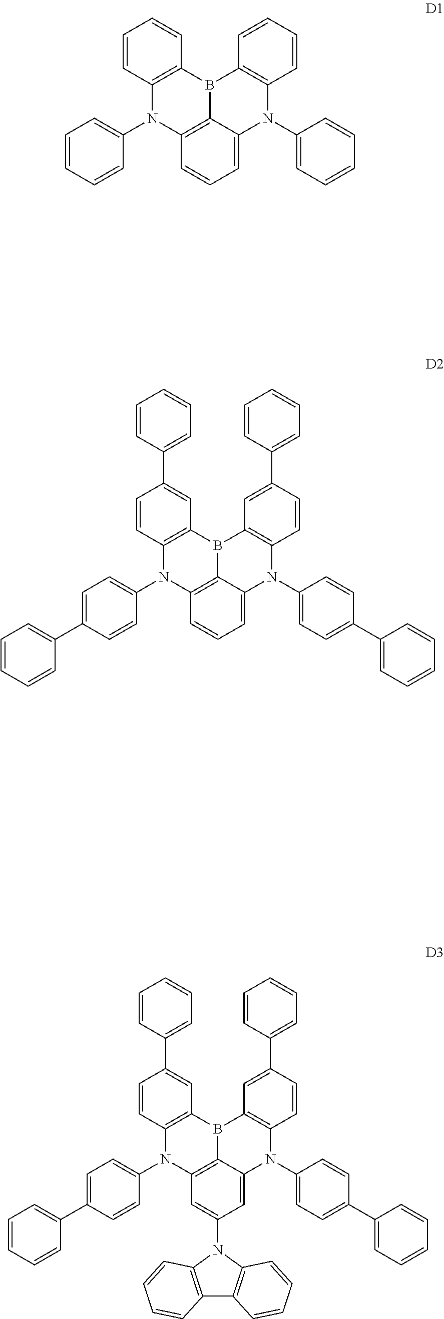 [SMILES: c1ccc(N2c3ccccc3B3c4ccccc4N(c4ccccc4)c4cccc2c43)cc1 ; c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-n6c7ccccc7c7ccccc76)cc3c54)cc2)cc1]

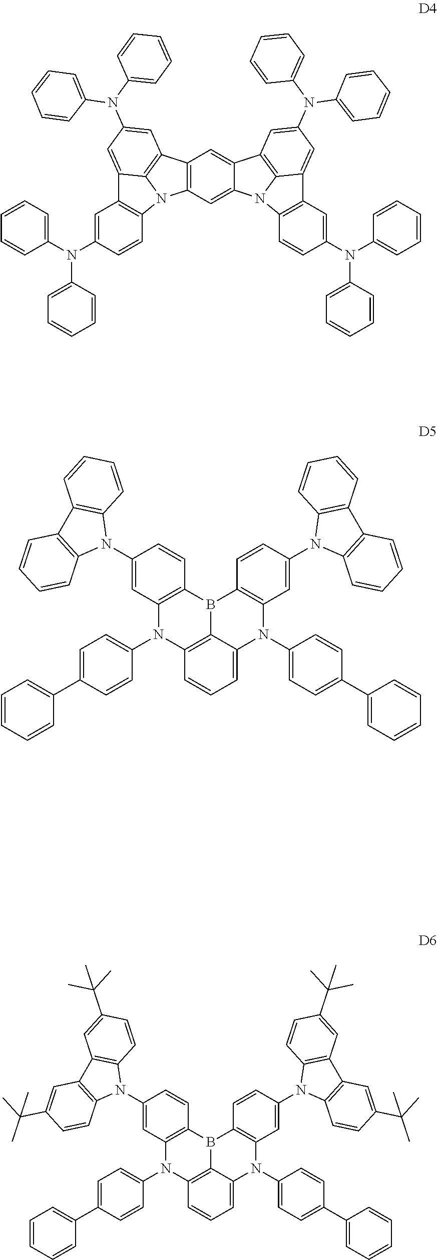 [SMILES: c1ccc(N(c2ccccc2)c2ccc3c(c2)c2cc(N(c4ccccc4)c4ccccc4)cc4c5cc6c7cc(N(c8ccccc8)c8ccccc8)cc8c9cc(N(c%10ccccc%10)c%10ccccc%10)ccc9n(c6cc5n3c24)c87)cc1 ; c1ccc(-c2ccc(N3c4cc(-n5c6ccccc6c6ccccc65)ccc4B4c5ccc(-n6c7ccccc7c7ccccc76)cc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ccc(-n2c4ccc(C(C)(C)C)cc4c4cc(C(C)(C)C)ccc42)cc1N3c1ccc(-c2ccccc2)cc1]

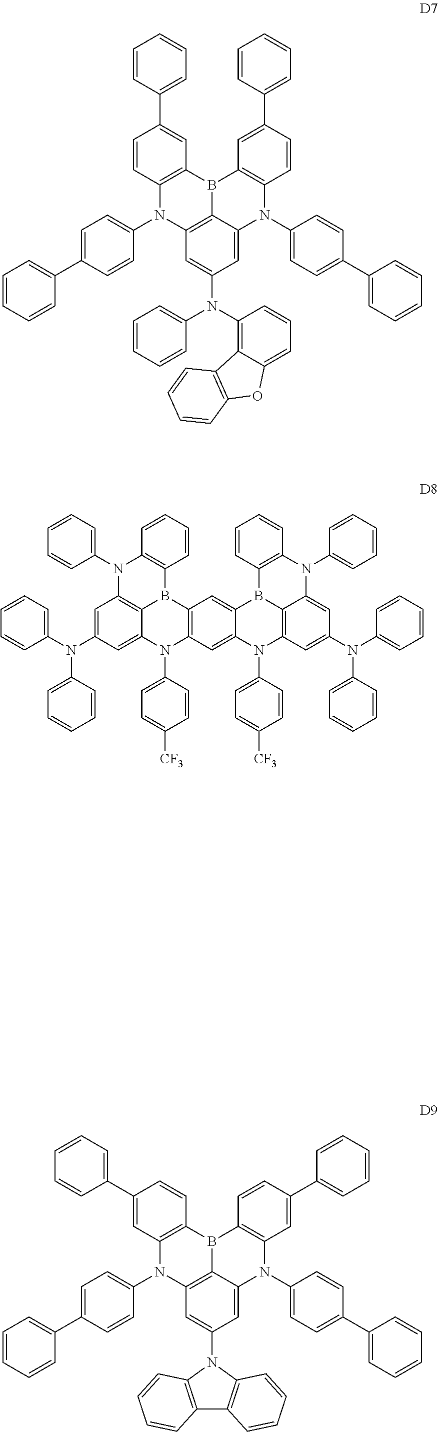 [SMILES: c1ccc(-c2ccc(N3c4ccc(-c5ccccc5)cc4B4c5cc(-c6ccccc6)ccc5N(c5ccc(-c6ccccc6)cc5)c5cc(N(c6ccccc6)c6cccc7oc8ccccc8c67)cc3c54)cc2)cc1 ; FC(F)(F)c1ccc(N2c3cc4c(cc3B3c5ccccc5N(c5ccccc5)c5cc(N(c6ccccc6)c6ccccc6)cc2c53)B2c3ccccc3N(c3ccccc3)c3cc(N(c5ccccc5)c5ccccc5)cc(c32)N4c2ccc(C(F)(F)F)cc2)cc1 ; c1ccc(-c2ccc(N3c4cc(-c5ccccc5)ccc4B4c5ccc(-c6ccccc6)cc5N(c5ccc(-c6ccccc6)cc5)c5cc(-n6c7ccccc7c7ccccc76)cc3c54)cc2)cc1]

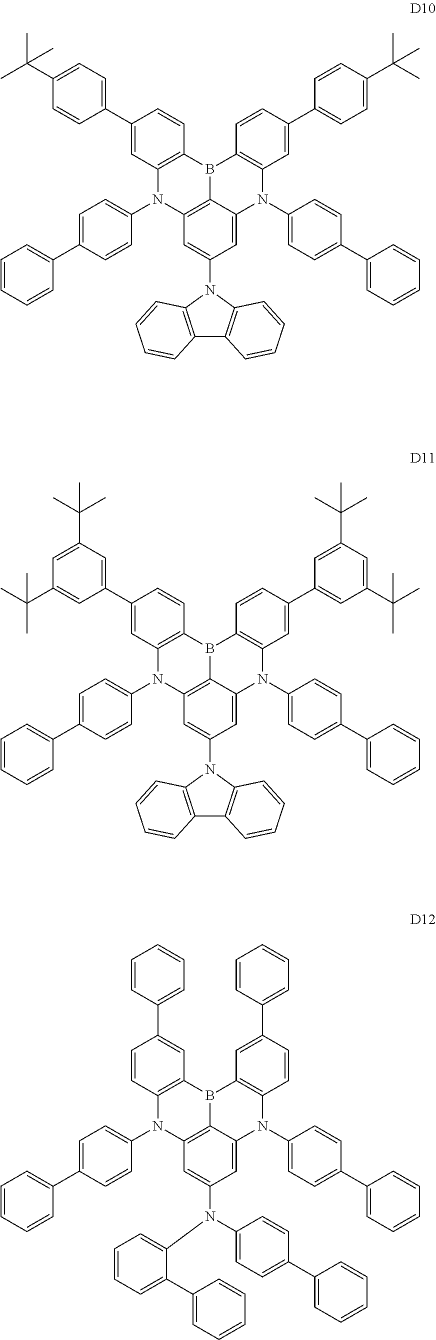 [SMILES: CC(C)(C)c1ccc(-c2ccc3c(c2)N(c2ccc(-c4ccccc4)cc2)c2cc(-n4c5ccccc5c5ccccc54)cc4c2B3c2ccc(-c3ccc(C(C)(C)C)cc3)cc2N4c2ccc(-c3ccccc3)cc2)cc1 ; CC(C)(C)c1cc(-c2ccc3c(c2)N(c2ccc(-c4ccccc4)cc2)c2cc(-n4c5ccccc5c5ccccc54)cc4c2B3c2ccc(-c3cc(C(C)(C)C)cc(C(C)(C)C)c3)cc2N4c2ccc(-c3ccccc3)cc2)cc(C(C)(C)C)c1 ; c1ccc(-c2ccc(N(c3cc4c5c(c3)N(c3ccc(-c6ccccc6)cc3)c3ccc(-c6ccccc6)cc3B5c3cc(-c5ccccc5)ccc3N4c3ccc(-c4ccccc4)cc3)c3ccccc3-c3ccccc3)cc2)cc1]

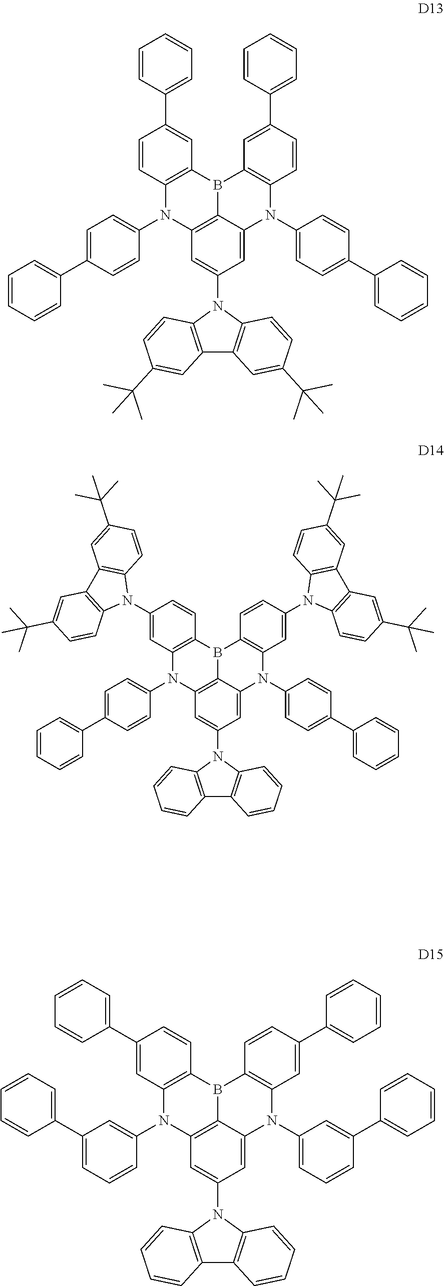 [SMILES: CC(C)(C)c1ccc2c(c1)c1cc(C(C)(C)C)ccc1n2-c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ccc(-n2c4ccc(C(C)(C)C)cc4c4cc(C(C)(C)C)ccc42)cc1N3c1ccc(-c2ccccc2)cc1 ; c1ccc(-c2cccc(N3c4cc(-c5ccccc5)ccc4B4c5ccc(-c6ccccc6)cc5N(c5cccc(-c6ccccc6)c5)c5cc(-n6c7ccccc7c7ccccc76)cc3c54)c2)cc1]

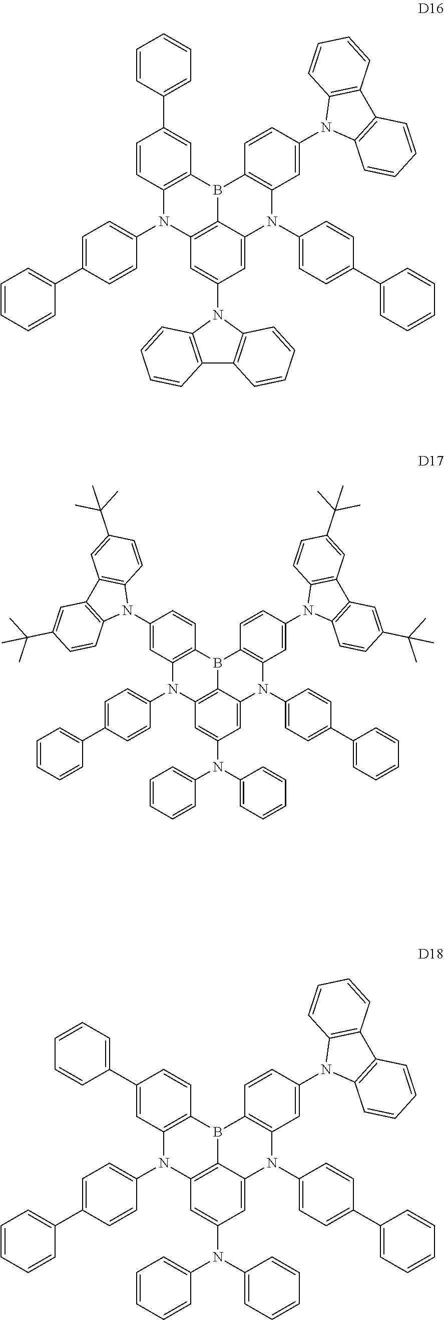 [SMILES: c1ccc(-c2ccc(N3c4ccc(-c5ccccc5)cc4B4c5ccc(-n6c7ccccc7c7ccccc76)cc5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1ccc(-n2c4ccc(C(C)(C)C)cc4c4cc(C(C)(C)C)ccc42)cc1N3c1ccc(-c2ccccc2)cc1 ; c1ccc(-c2ccc(N3c4cc(-c5ccccc5)ccc4B4c5ccc(-n6c7ccccc7c7ccccc76)cc5N(c5ccc(-c6ccccc6)cc5)c5cc(N(c6ccccc6)c6ccccc6)cc3c54)cc2)cc1]

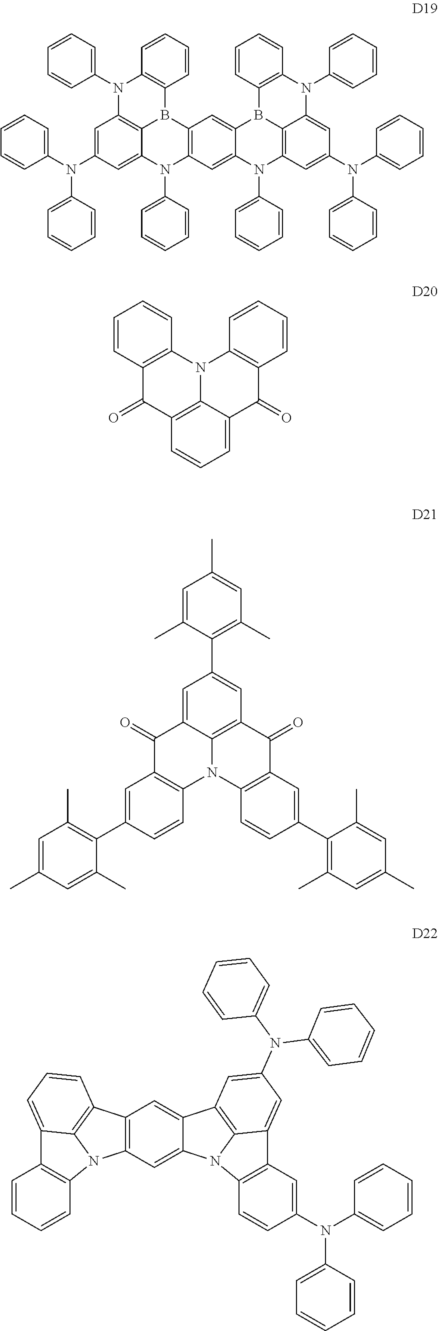

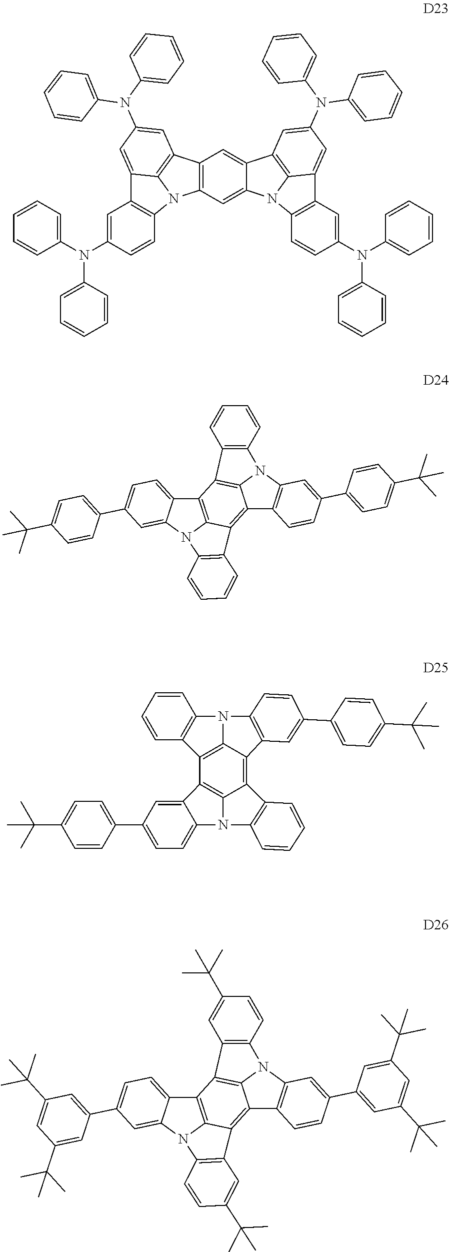

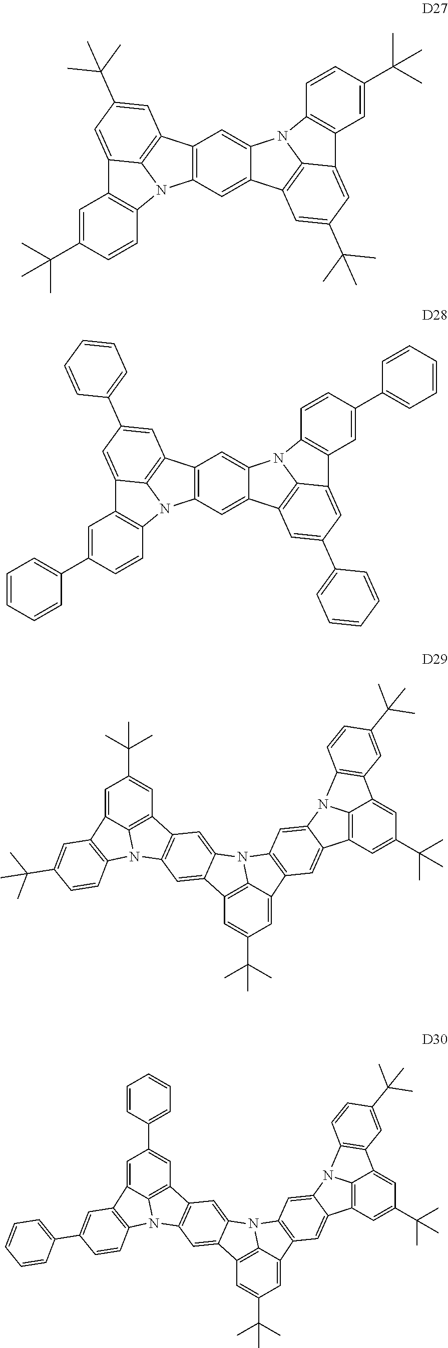

The phosphorescent sensitizer and the florescent dopant may be included in an amount of less than or equal to about 20 wt %, e.g., about 0.1 wt % to about 20 wt %, about 0.1 wt % to about 15 wt %, about 0.1 wt % to about 10 wt %, about 0.1 wt % to about 7 wt %, about 0.1 wt % to about 5 wt %, about 0.1 wt % to about 4 wt %, about 1 wt % to about 20 wt %, about 1 wt % to about 15 wt %, about 1 wt % to about 10 wt %, about 1 wt % to about 7 wt %, about 1 wt % to about 5 wt %, or about 1 wt % to about 4 wt %, based on a total amount of the composition for an organic optoelectronic device.

For the light-emitting dopant, a suitable compound may be used.

The composition for an organic optoelectronic device may further include an additive, and the additive may be, e.g., an organic material, an inorganic material, an organic/inorganic material, or a combination thereof.

Hereinafter, an organic optoelectronic device using the aforementioned composition for an organic optoelectronic device will be described.

The organic optoelectronic device may be, e.g., an organic light emitting diode, an organic photoelectric device, or an organic solar cell. In an implementation, an organic optoelectronic device may be an organic light emitting diode.

The organic optoelectronic device may include an anode and a cathode facing each other, and an organic layer between the anode and the cathode, and the organic layer may include the aforementioned composition. The organic layer may include an active layer, e.g., a light emitting layer or a light absorbing layer, and the aforementioned composition may be included in the active layer. The organic layer may include an auxiliary layer between the anode and the active layer or between the cathode and the active layer, and the aforementioned composition may be included in the auxiliary layer.

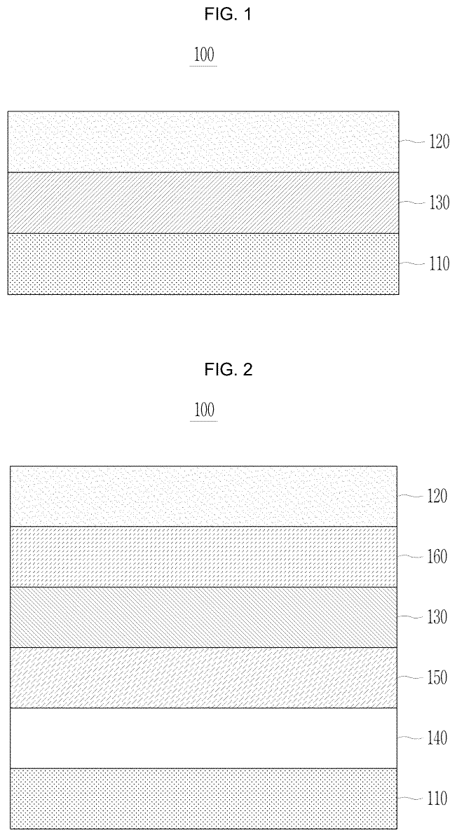

FIG. 1 is a cross-sectional view showing an example of an organic light emitting diode, which may be an example of an organic optoelectronic device according to an embodiment.

Referring to FIG. 1, the organic light emitting diode 100 according to an embodiment may include an anode 110 and a cathode 120 facing each other, and a light emitting layer 130 between the anode 110 and the cathode 120.

The anode 110 may be made of a conductor with a high work function to facilitate hole injection, e.g., and may be made of a metal, a metal oxide, or a conductive polymer. The anode 110 may be made of a metal, e.g., nickel, platinum, vanadium, chromium, copper, zinc, gold, or an alloy thereof, a metal oxide, e.g., zinc oxide, indium oxide, indium tin oxide (ITO), and indium zinc oxide (IZO); a combination of a metal and an oxide, e.g., ZnO and Al or SnO₂ and Sb; a conductive polymer, e.g., poly(3-methylthiophene), poly(3,4-(ethylene-1,2-dioxy)thiophene) (PEDOT), polypyrrole, and polyaniline.

The cathode 120 may be made of a conductor with a low work function to facilitate electron injection, e.g., and may be made of a metal, a metal oxide, or a conductive polymer. The cathode 120 may be made of a metal, e.g., magnesium, calcium, sodium, potassium, titanium, indium, yttrium, lithium, gadolinium, aluminum, silver, tin, lead, cesium, barium, or an alloy thereof, a multilayer structure material, e.g., LiF/Al, LiO₂/Al, LiF/Ca, or BaF₂/Ca.

The light emitting layer 130 may include the aforementioned composition for organic optoelectronic devices as a mixed host. The light emitting layer 130 may further include another organic compound as a mixed host. The light emitting layer 130 may further include the aforementioned light-emitting dopant and may include a fluorescent dopant, a phosphorescent sensitizer, or a combination thereof as described above. In an implementation, the light emitting layer 130 may emit light in a blue light-emitting spectrum by combining the aforementioned composition for an organic optoelectronic device and a light-emitting dopant. At least one of the aforementioned first and second compounds of the composition for an organic optoelectronic device may have a high triplet energy level of greater than or equal to about 2.8 eV, so that exciton transfer to the blue light-emitting dopant may be easy, and thus an organic optoelectronic device having high-efficiency and long life-span may be realized. The peak wavelength of the blue emission spectrum may fall within, e.g., about 410 nm to about 480 nm, e.g., about 420 nm to about 470 nm or about 430 nm to about 470 nm.

In an implementation, the aforementioned composition for an organic optoelectronic device may include a first compound, which may be a bipolar compound with relatively strong electron transport characteristics, and a second compound, which may be a compound with relatively strong hole transport characteristics, so that luminous efficiency may be increased by increasing a balance of electrons and holes within the light emitting layer 130, and at the same time life-span may be improved by reducing the non-combined charges due to the imbalance in the mobility of electrons and holes, compared to the case where the first compound alone or the second compound alone is used.

In an implementation, in the organic light emitting diode 100 including the light emitting layer 130 using the composition for organic optoelectronic devices as a mixed host, the holes and electrons injected from the anode 110 and the cathode 120 may appropriately be distributed within the light emitting layer 130, the mobility may be finely controlled to an appropriate level, and exciton generation within the light emitting layer 130 may be strongly induced, thereby increasing the light emitting efficiency of the light emitting layer 130.

In an implementation, due to a difference in mobility within the light emitting layer 130 of holes and electrons injected from the anode 110 and the cathode 120, respectively, generation of exciton at inappropriate locations, e.g., the interface between the light emitting layer 130 and adjacent layers, or accumulation of non-combined charges at the interface between the light emitting layer 130 and adjacent layers may be reduced or prevented.

In an implementation, it may be possible to reduce or prevent the roll-off phenomenon in which the luminous efficiency of the organic light emitting diode 100 rapidly decreases due to non-luminescent excitons or non-combined charges, thus ultimately improving life-span of the organic light emitting diode 100.

The organic light emitting diode 100 may be manufactured by forming an anode 110 or a cathode 120 on a substrate, forming a light emitting layer using dry film forming methods, e.g., vacuum evaporation, sputtering, plasma plating, and ion plating, and forming a cathode 120 or an anode 110 thereon.

FIG. 2 is a cross-sectional view showing another example of an organic light emitting diode, which may be an example of an organic optoelectronic device according to another embodiment.

Referring to FIG. 2, the organic light emitting diode 100 according to another embodiment may include an anode 110, a cathode 120, and a light emitting layer 130, similar to the aforementioned embodiment. However, unlike the aforementioned embodiment, the organic light emitting diode 100 according to another embodiment may further include a hole transport layer 140, a hole transport auxiliary layer 150, and an electron transport layer 160.

The hole transport layer 140 may be located between the anode 110 and the light emitting layer 130, and the hole transport auxiliary layer 150 may be located between the light emitting layer 130 and the hole transport layer 140. The electron transport layer 160 may be located between the cathode 120 and the light emitting layer 130.

The hole transport layer 140 may facilitate hole transfer from the anode 110 to the light emitting layer 130, and may include, e.g., an amine compound. In an implementation, the amine compound may have at least one aryl group or heteroaryl group with hole characteristics. In an implementation, the amine compound may be represented by Chemical Formula 6a or 6b.

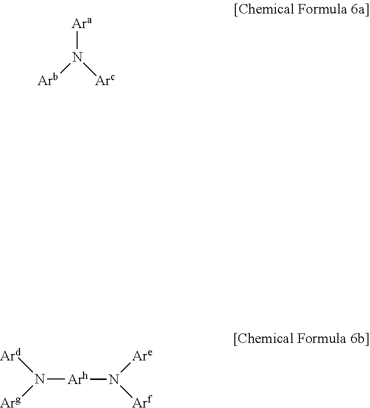

In Chemical Formula 6a or 6b, Ar^{a} to Ar^{g} may each independently be or include, e.g., hydrogen, deuterium, a substituted or unsubstituted C1 to C20 alkyl group, a substituted or unsubstituted C6 to C30 aryl group, a substituted or unsubstituted C2 to C30 heteroaryl group, or a combination thereof.

At least one of Ar^{a} to Ar^{c} and at least one of Ar^{d} to Ar^{g} may be, e.g., a substituted or unsubstituted C6 to C30 aryl group, a substituted or unsubstituted C2 to C30 heteroaryl group, or a combination thereof.

Ar^{h} may be, e.g., a single bond, a substituted or unsubstituted C1 to C20 alkylene group, a substituted or unsubstituted C6 to C30 arylene group, a substituted or unsubstituted C2 to C30 heteroarylene group, or a combination thereof.

The hole transport auxiliary layer 150 may form an interface with the light emitting layer 130 by being located between the hole transport layer 140 and the light emitting layer 130 and in contact with the light emitting layer 130. The hole transport auxiliary layer 150 may further reduce or prevent the generation of exciton at inappropriate locations, e.g., the interface between the aforementioned light emitting layer 130 and adjacent layers or the accumulation of non-combined charges at the interface between the light emitting layer 130 and adjacent layers. In an implementation, it may be possible to further reduce or prevent the roll-off phenomenon in which the luminous efficiency of the organic light emitting diode 100 rapidly decreases due to non-luminescent excitons or unbound charges, and thus ultimately improve the life-span of the organic light emitting diode 100.

The electron transport layer 160 may further increase electron injection or electron mobility and block holes between the cathode 120 and the light emitting layer 130.

The electron transport layer 160 may include, e.g., a compound of Group 5.

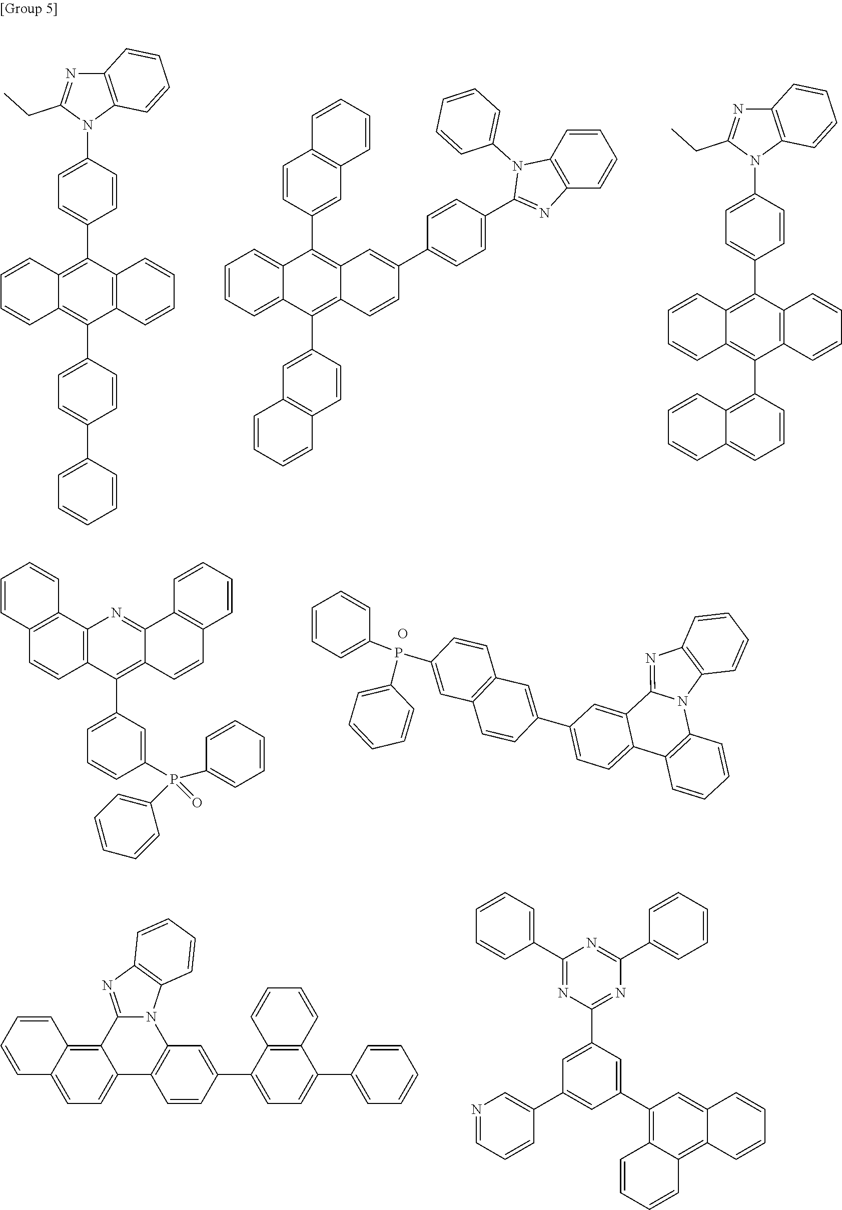

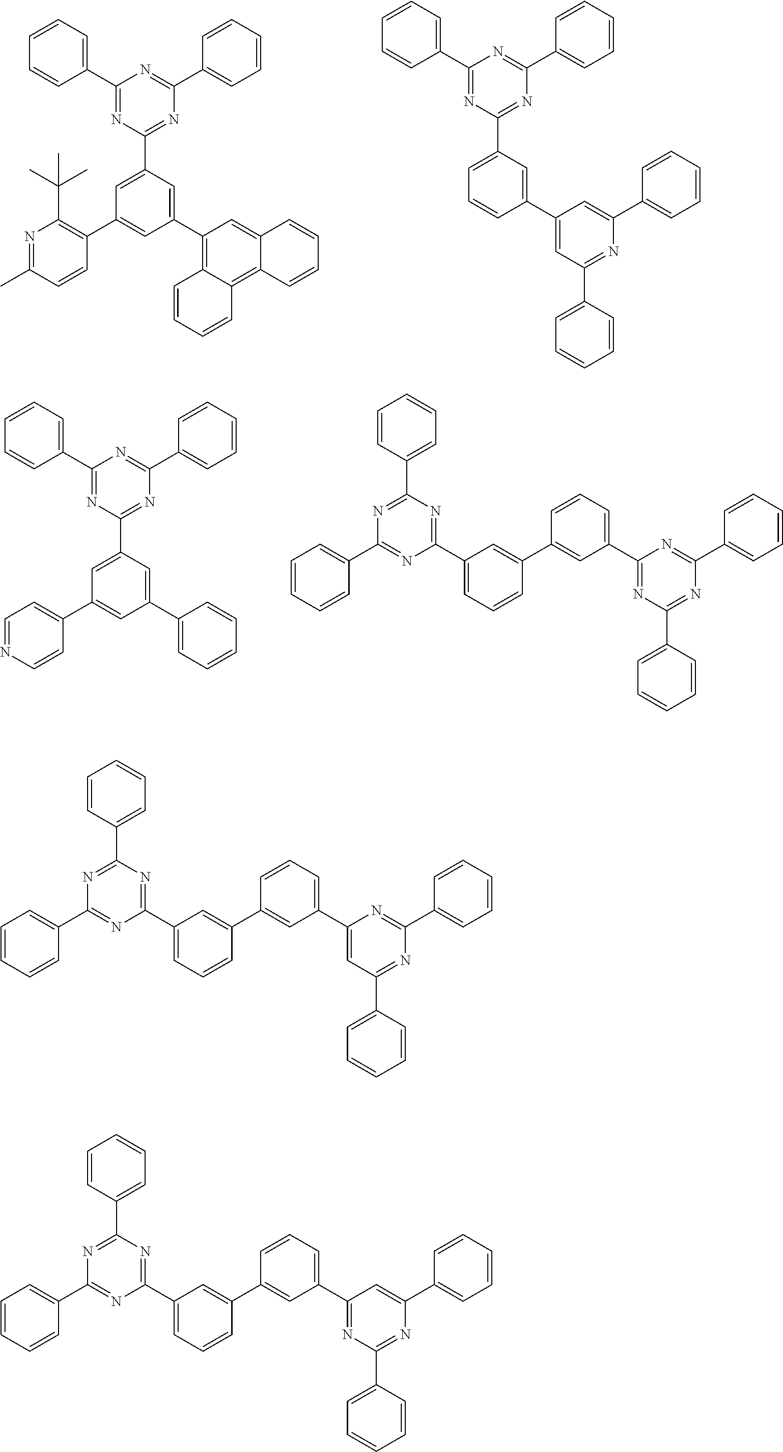 [SMILES: Cc1ccc(-c2cc(-c3nc(-c4ccccc4)nc(-c4ccccc4)n3)cc(-c3cc4ccccc4c4ccccc34)c2)c(C(C)(C)C)n1 ; c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccncc3)cc(-c3nc(-c4ccccc4)nc(-c4ccccc4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

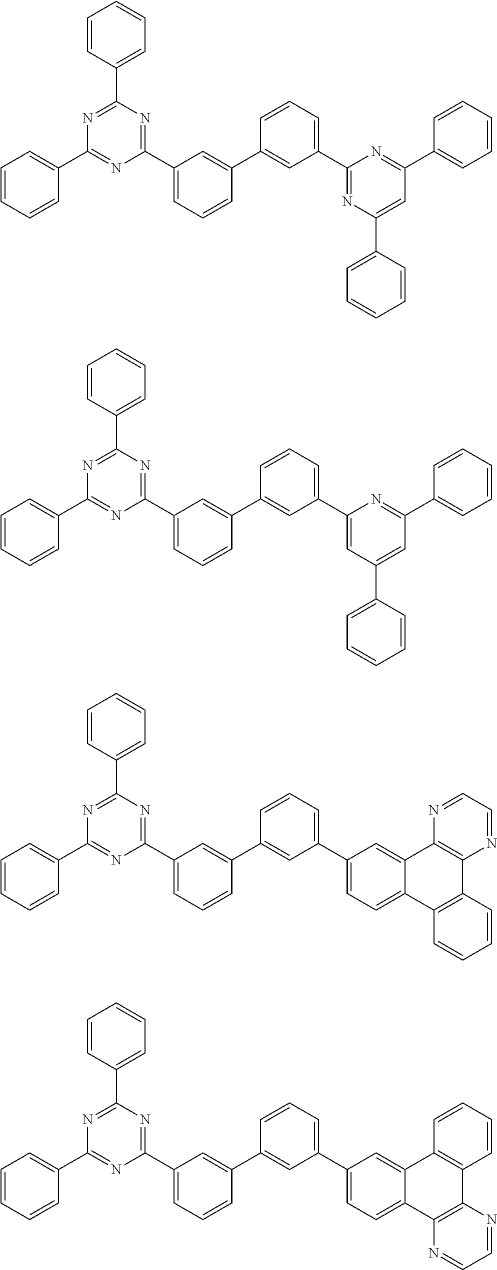 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

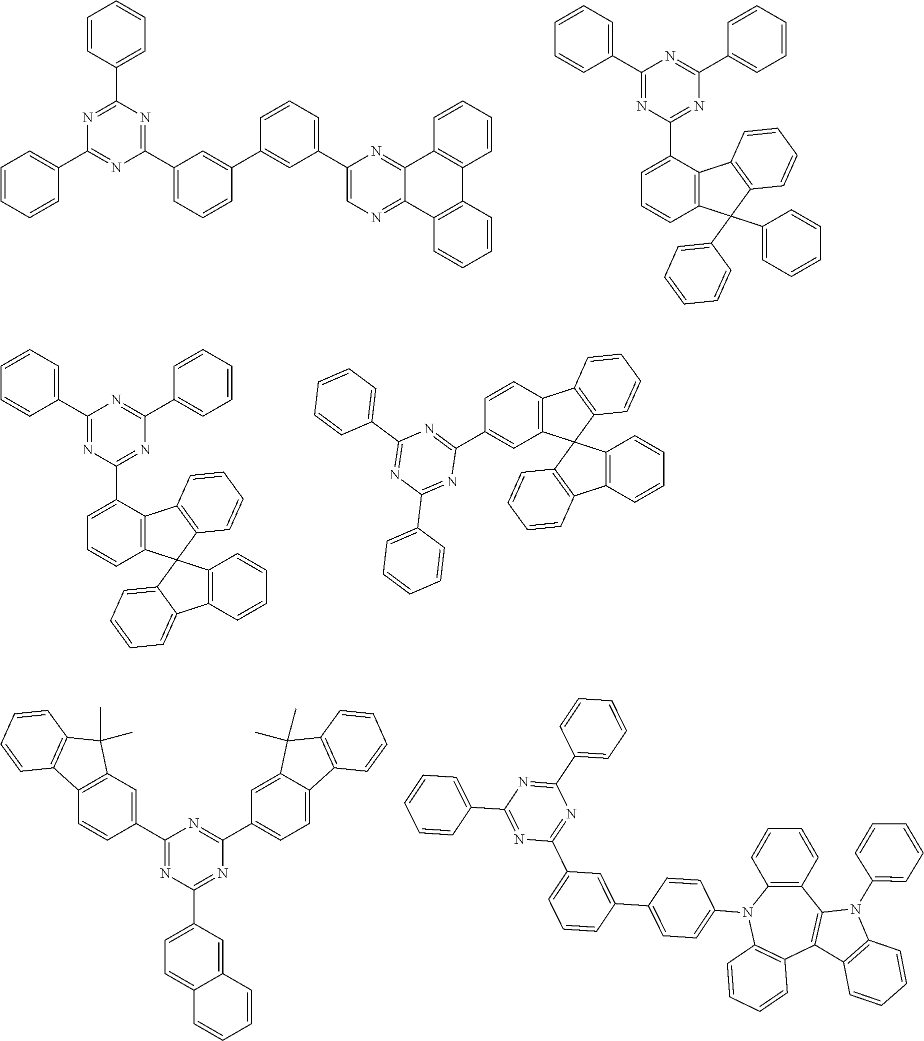 [SMILES: c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1 ; c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3ccc4c(c3)C3(c5ccccc5-c5ccccc53)c3ccccc3-4)n2)cc1 ; CC1(C)c2ccccc2-c2ccc(-c3nc(-c4ccc5c(c4)C(C)(C)c4ccccc4-5)nc(-c4ccc5ccccc5c4)n3)cc21 ; c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1]

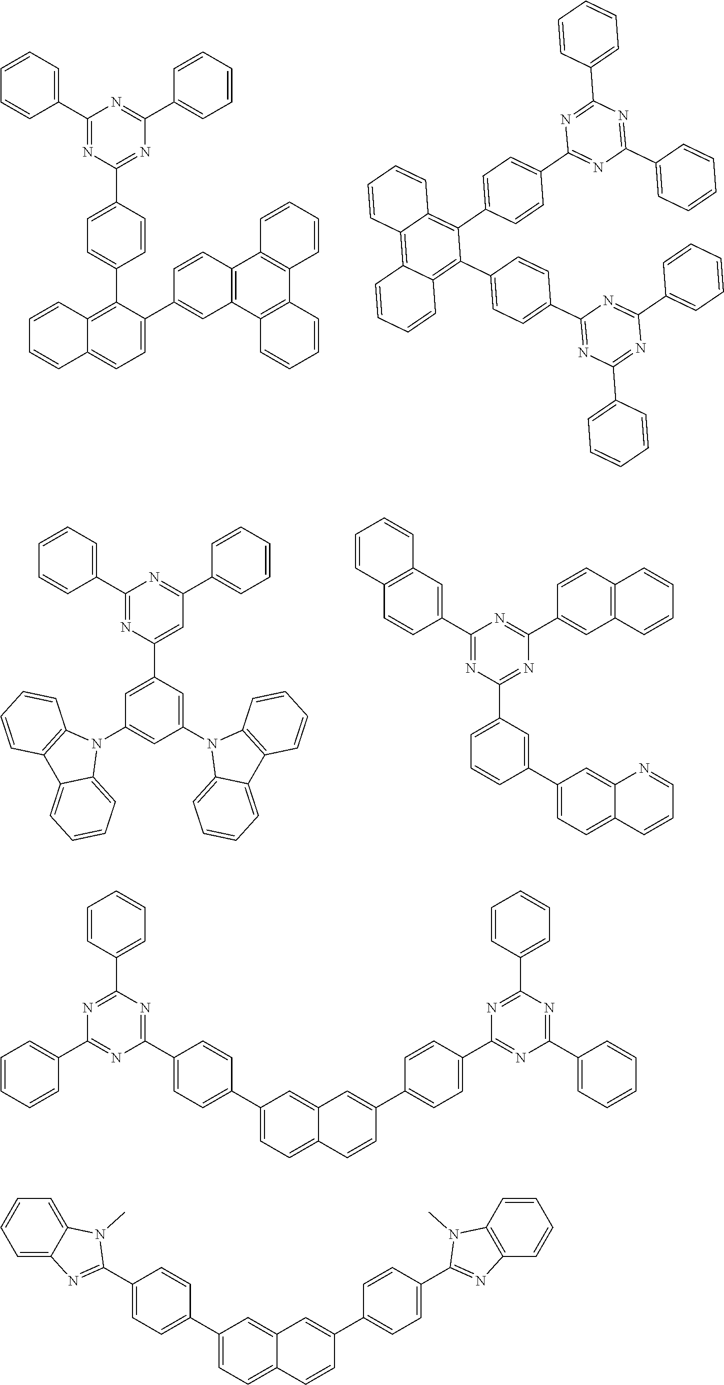

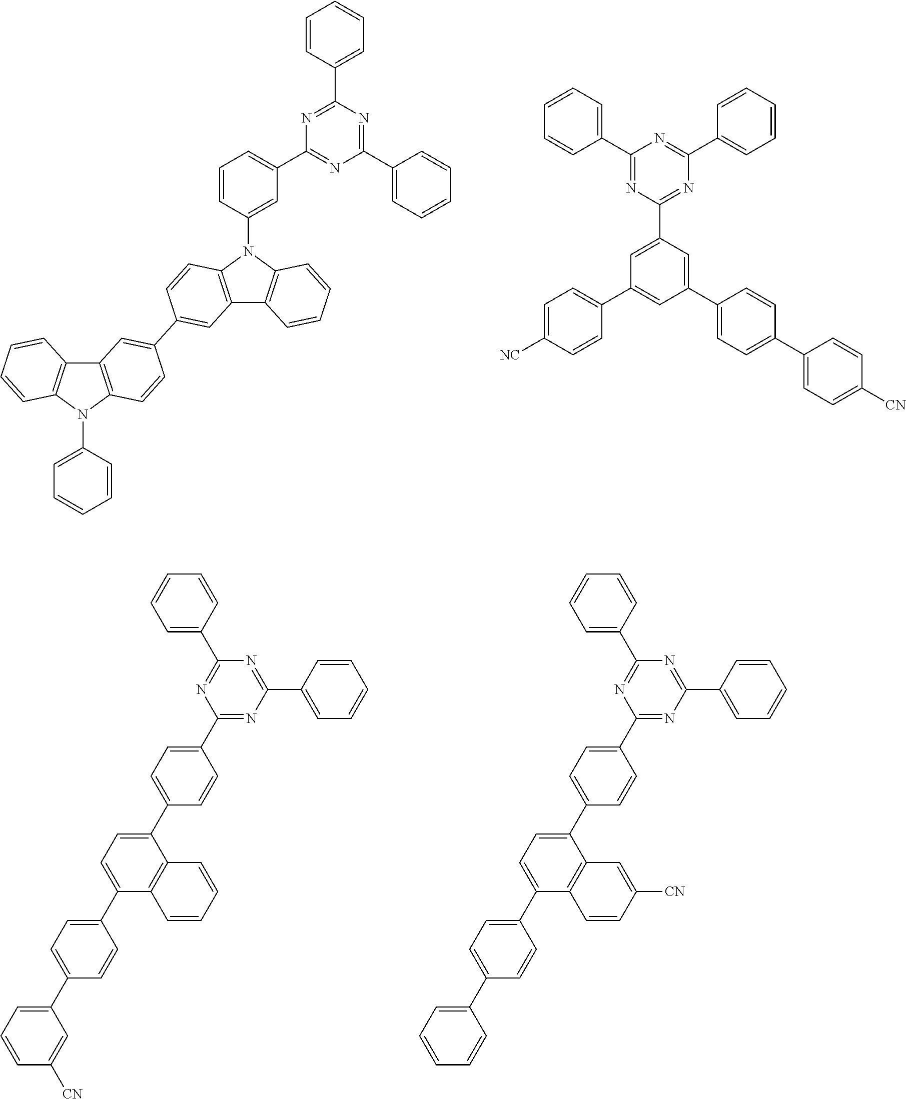 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

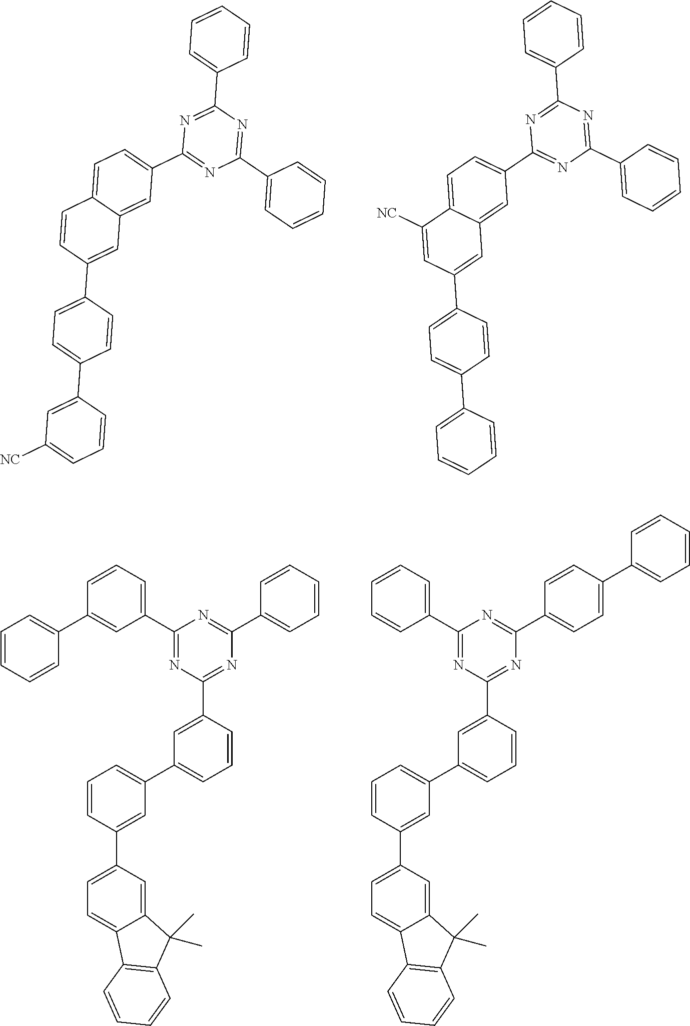 [SMILES: N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12 ; CC1(C)c2ccccc2-c2ccc(-c3cccc(-c4cccc(-c5nc(-c6ccccc6)nc(-c6cccc(-c7ccccc7)c6)n5)c4)c3)cc21 ; CC1(C)c2ccccc2-c2ccc(-c3cccc(-c4cccc(-c5nc(-c6ccccc6)nc(-c6ccc(-c7ccccc7)cc6)n5)c4)c3)cc21]

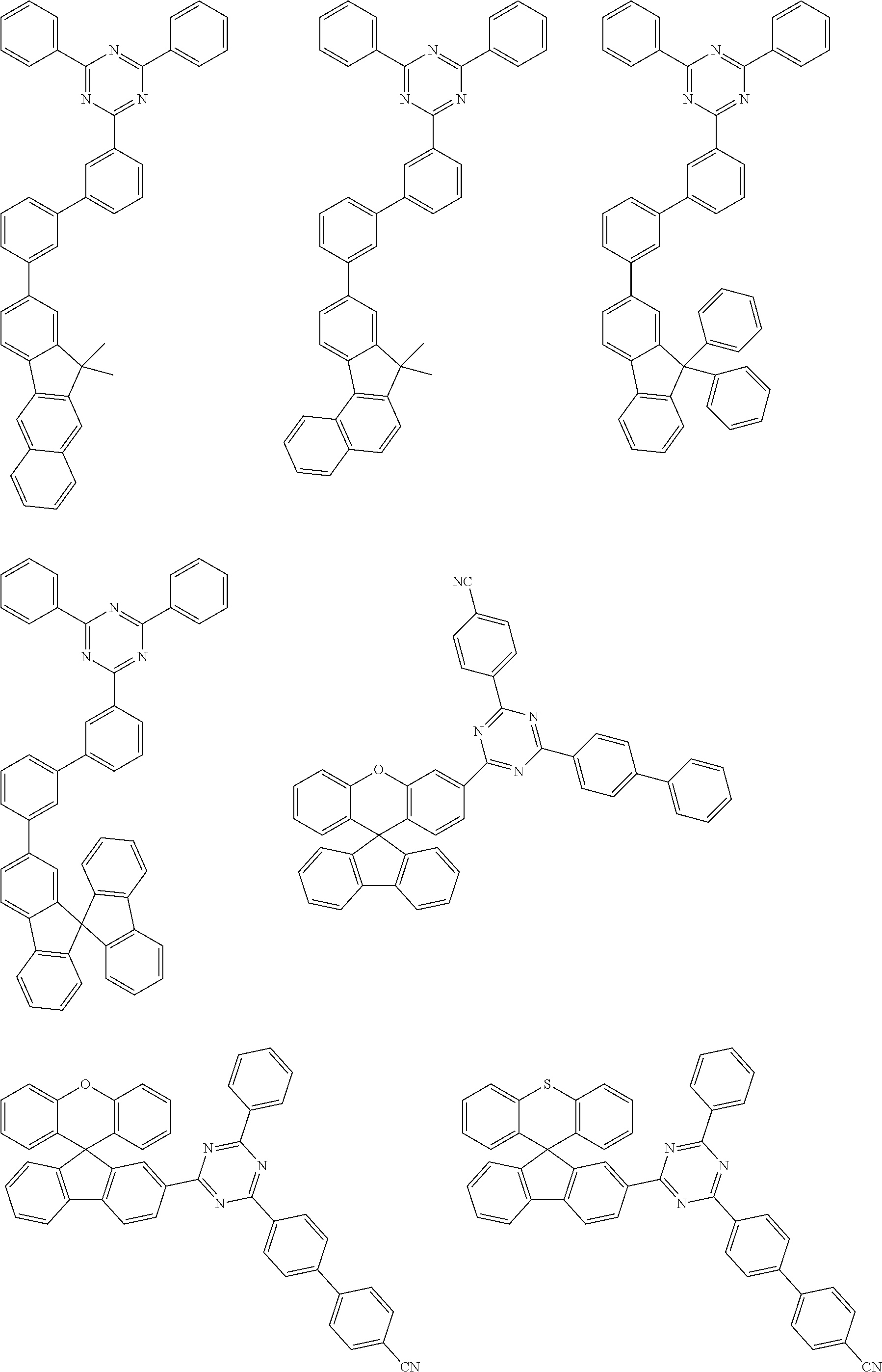

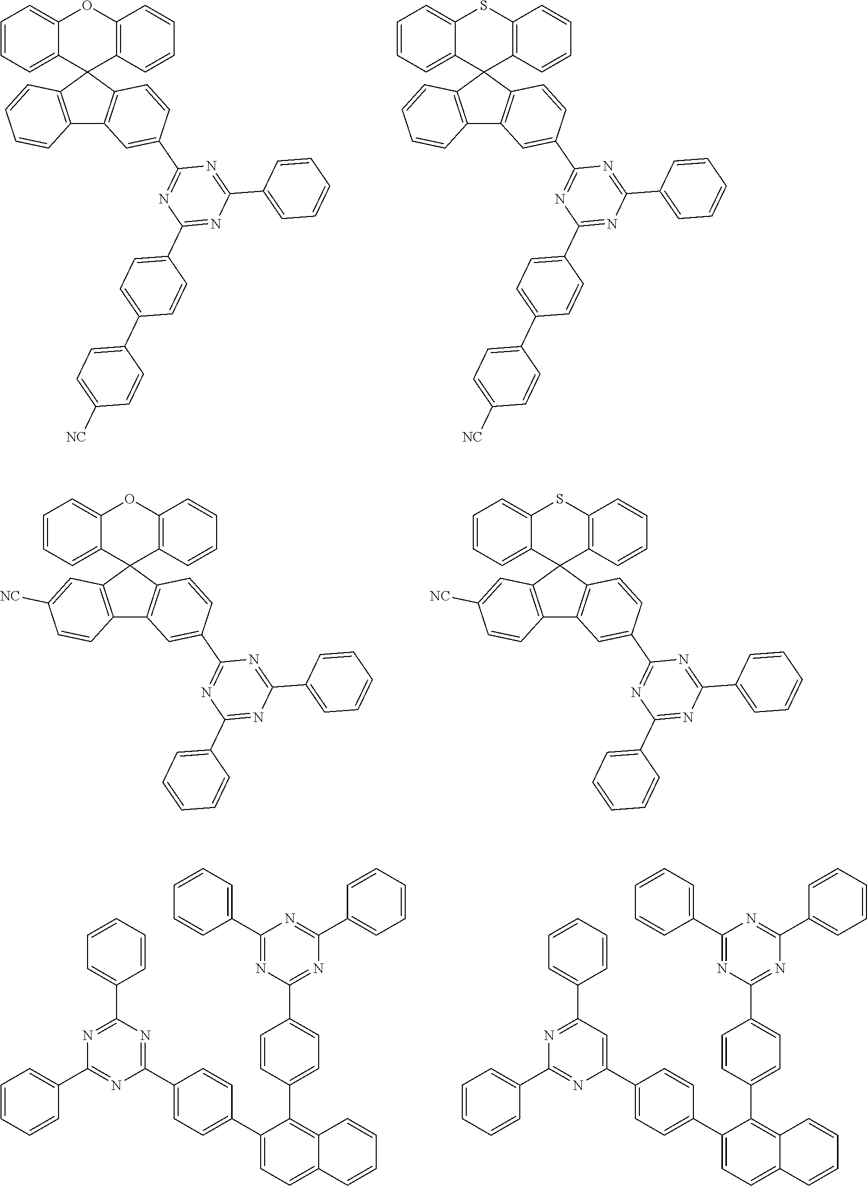

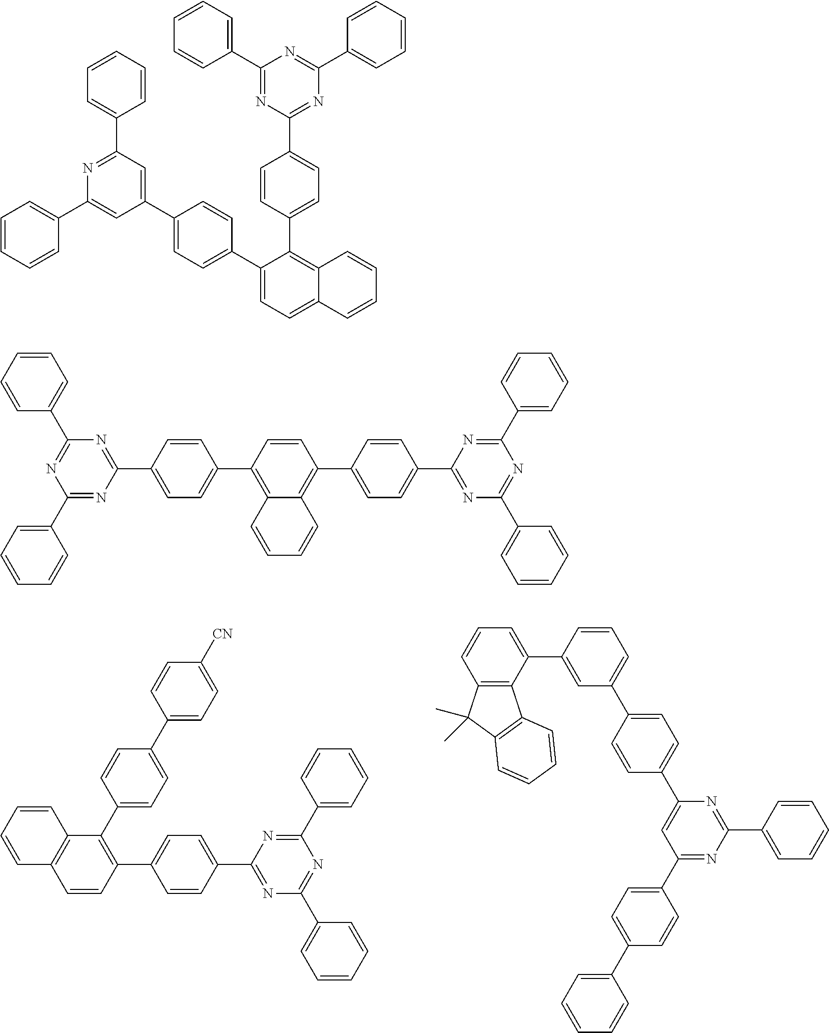

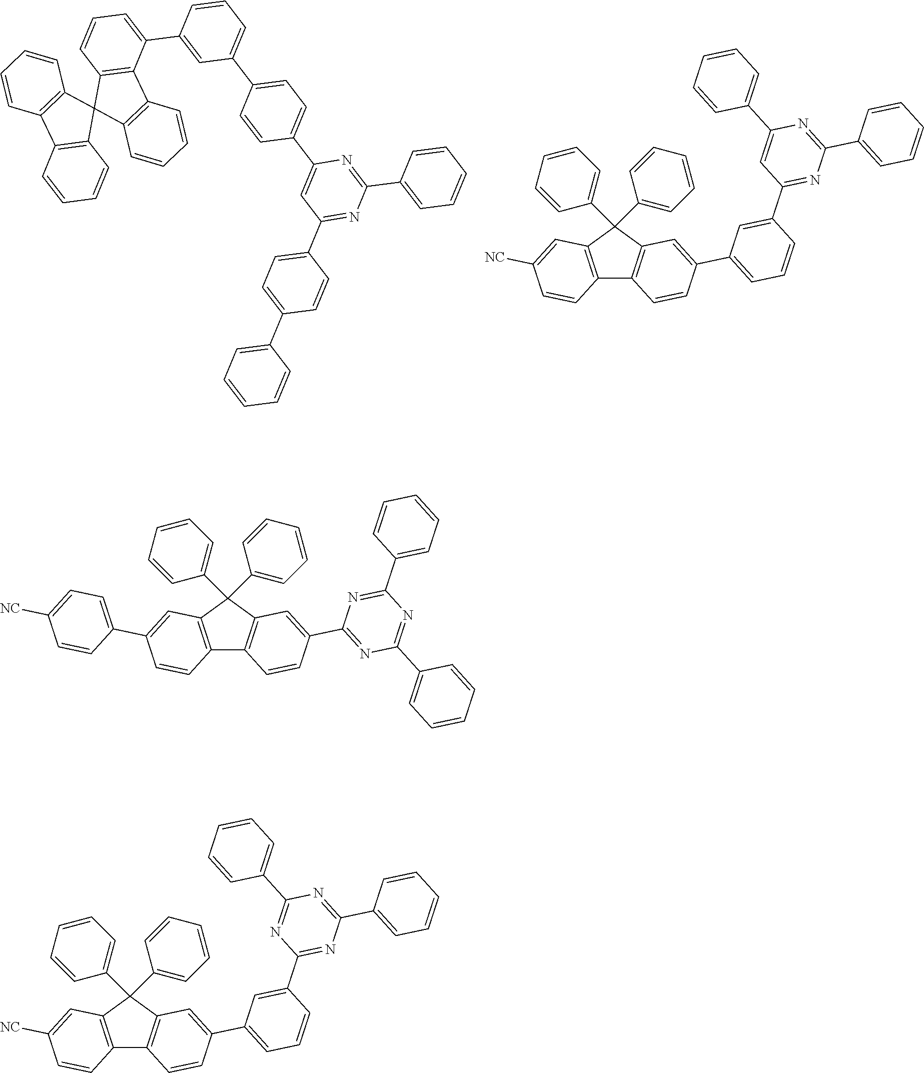 [SMILES: c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1 ; N#Cc1ccc2c(c1)C(c1ccccc1)(c1ccccc1)c1cc(-c3cccc(-c4cc(-c5ccccc5)nc(-c5ccccc5)n4)c3)ccc1-2 ; N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

The organic optoelectronic device, including the aforementioned organic light emitting diodes, may be applied to display devices.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry or P&H tech as far as there is no particular comment or were synthesized by known methods.

### Preparation of Compound for Organic Optoelectronic Device

The compounds were synthesized through the following steps.

### Synthesis of First Compound

### Synthesis Example 1: Synthesis of Compound A-54

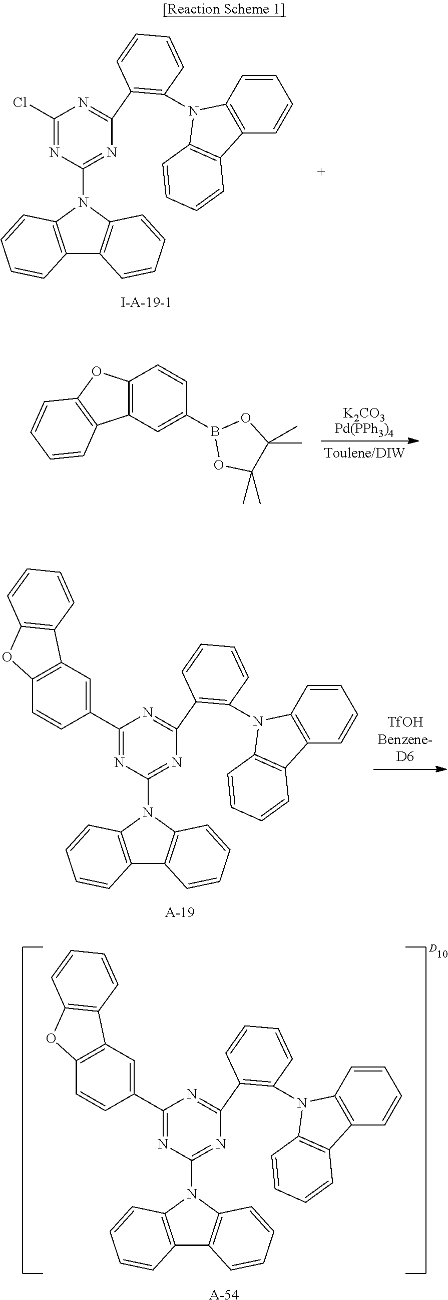

### 1st Step: Synthesis of Compound A-19

Compound A-19 was synthesized using Intermediates I-A-19-1 (CAS No. 2920056-52-4), 2-(dibenzofuran-2-yl)-4,4,5,5-tetramethyl-1,3,2-dioxaborolane were prepared by referring to the synthesis method described in Korean Patent Publication No. 10-2023-0037447.

### 2nd Step: Synthesis of Compound A-54

Compound A-19, trifluoromethanesulfonic acid, and benzene-D₆ were added and stirred for 24 hours. Purified water was added and neutralized with a saturated K₃PO₄ solution. The organic layer was concentrated and purified by column to synthesize Compound A-54.

### Synthesis Example 2: Synthesis of Compound A-55

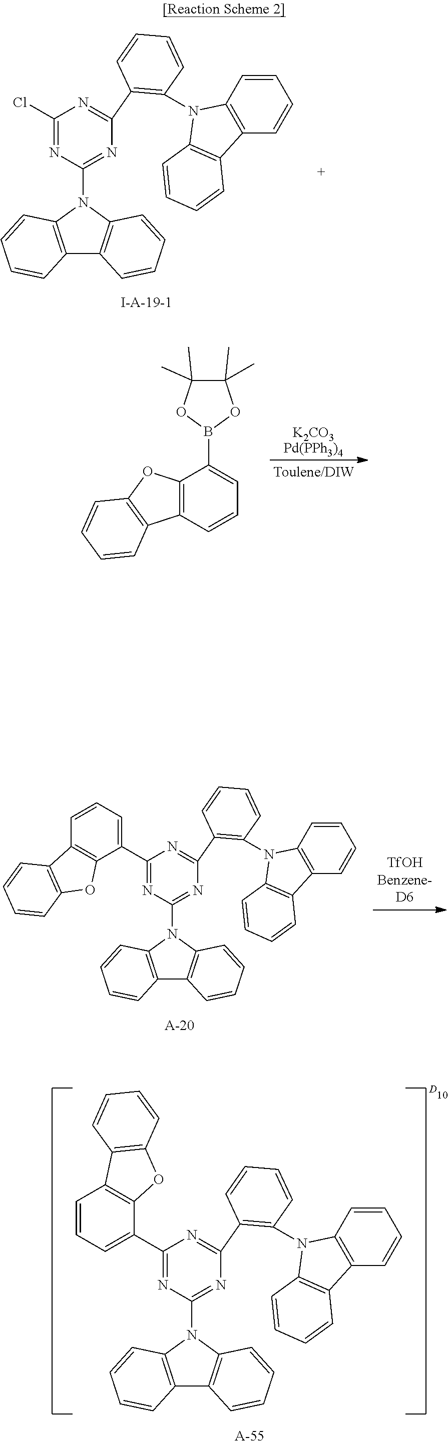

### 1st Step: Synthesis of Compound A-20

Compound A-20 was synthesized using Intermediates I-A-19-1 (CAS No. 2920056-52-4), and 2-(dibenzofuran-4-yl)-4,4,5,5-tetramethyl-1,3,2-dioxaborolane by referring to the synthesis method described in Korean Patent Publication No. 10-2023-0037447.

### 2nd Step: Synthesis of Compound A-55

Compound A-20, trifluoromethanesulfonic acid, and benzene-D₆ were added and stirred for 24 hours. Purified water was added and neutralized with a saturated K₃PO₄ solution. The organic layer was concentrated and purified by column to synthesize Compound A-55.

### Synthesis of Second Compound

### Synthesis Example 3: Synthesis of Compound D-72

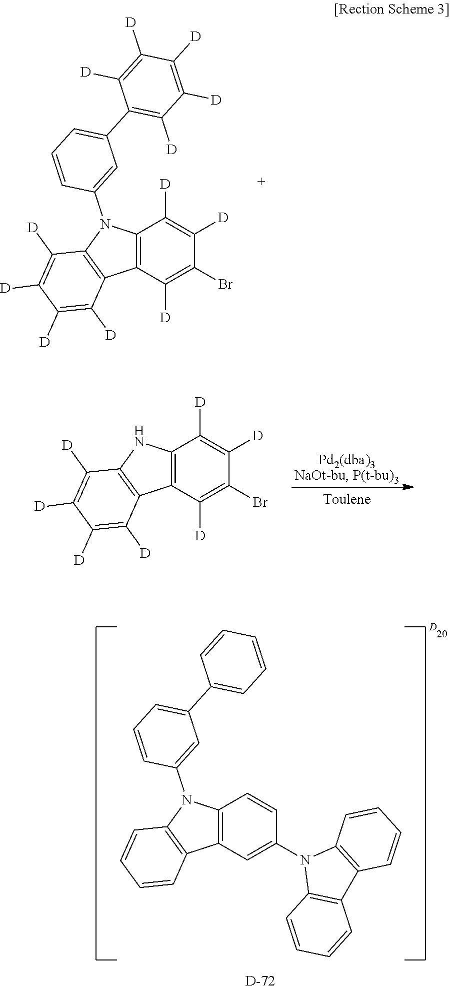

Compound D-72 was synthesized by referring to the synthesis method described in Korean Patent Publication No. 10-2023-0155972.

### Comparative Synthesis Example 1: Synthesis of Compound HT-1

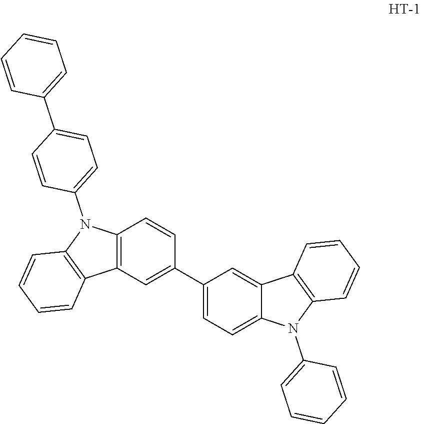 [SMILES: c1ccc(-c2ccc(-n3c4ccccc4c4cc(-c5ccc6c(c5)c5ccccc5n6-c5ccccc5)ccc43)cc2)cc1]

Comparative compound HT-1 was synthesized by referring to the synthesis method described in Korean Patent Publication No. 10-2023-0037447.

### Manufacture of Organic Light Emitting Diode

### Example 1

A glass substrate coated with a thin film of ITO (indium tin oxide) was ultrasonically washed with distilled water. After washing with the distilled water, the glass substrate was ultrasonically washed with isopropyl alcohol, acetone, or methanol and dried and then, moved to a plasma cleaner, cleaned by using oxygen plasma for 10 minutes, and moved to a vacuum depositor. The prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 600 Å to form a hole transport layer. mCP was deposited to a thickness of 100 Å on the hole transport layer to form a hole transport auxiliary layer. On the hole transport auxiliary layer, Compound A-54 obtained in Synthesis Example 1 and Compound D-72 synthesized in Synthesis Example 3 were used simultaneously as hosts, P31 was doped at 13 wt % as a phosphorescent sensitizer, and D3 was doped at 1.5 wt % as a fluorescent dopant to form a 400 Å-thick light emitting layer by vacuum deposition. Herein, Compound A-54 and Compound D-72 were used in a weight ratio of 4:6. Subsequently, BCP was deposited on the light emitting layer to a thickness of 50 Å to form an electron transport auxiliary layer, and Compound B and Liq were simultaneously vacuum deposited in a weight ratio of 1:1 to form a 300 Å-thick electron transport layer. An organic light emitting diode was manufactured by sequentially vacuum depositing 10 Å of Liq and 1,200 Å of Al on the electron transport layer to form a cathode.

ITO/Compound A (3% NDP-9 doping, 100 Å)/Compound A (600 Å)/mCP (100 Å)/EML [Host (Compound A-54: Compound D-72):P31:D3=85.5 wt %: 13 wt %: 1.5 wt %](400 Å)/BCP (50 Å)/Compound B:Liq (300 Å)/Liq (10 Å)/Al (1,200 Å).
Compound A: N-(9,9-diphenyl-9H-fluoren-2-yl)-N,9-diphenyl-9H-carbazol-2-amine
Compound B: 8-{4-[bis(naphthalene-2-yl)-1,3,5-triazin-2-yl]phenyl}quinoline

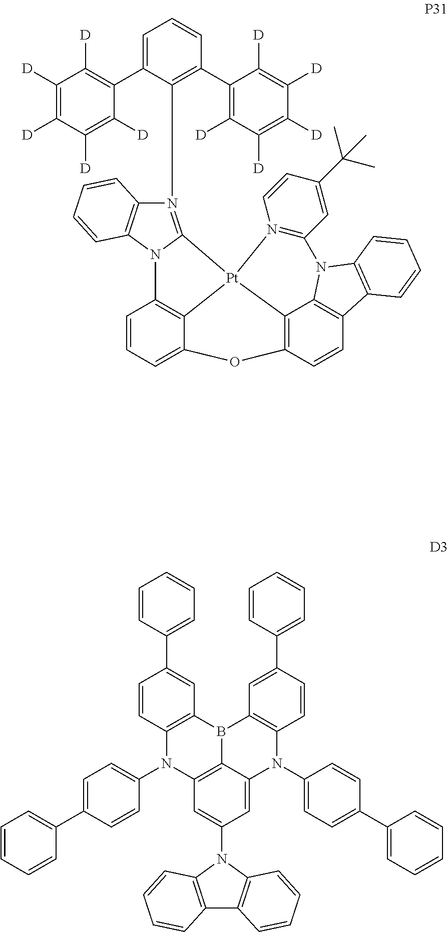

### Example 2

An organic light emitting diode was manufactured in the same manner as in Example 1 except that Compound A-55 obtained in Synthesis Example 2 and Compound D-72 obtained in Synthesis Example 3 were used as the host of the light emitting laver instead of Compound A-54 obtained in Synthesis Example 1 and Compound D-72 synthesized in Synthesis Example 3 to form the light emitting layer.

### Comparative Example 1

An organic light emitting diode was manufactured in the same manner as in Example 1 except that Compound A-54 obtained in Synthesis Example 1 and Compound HT-1 obtained in Comparative Synthesis Example 1 were used as the host of the light emitting layer, instead of Compound A-54 obtained in Synthesis Example 1 and Compound D-72 synthesized in Synthesis Example 3 to form the light emitting layer.

### Evaluation

The luminous efficiency and life-span characteristics of organic light emitting diodes according to Examples 1 and 2 and Comparative Example 1 were evaluated.

The specific measurement method is as follows, and the results are in Table 1.

### (1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

Current efficiency (cd/A) at the same current density (10 mA/cm²) was calculated by using the luminance and current density from (1) and (2) above and voltage.

The luminous efficiency values of Examples 1 and 2 and Comparative Example 1 were calculated as relative values based on Comparative Example 1 and are shown in Table 1.

### (4) Measurement of Life-Span

The results were obtained by maintaining the luminance (cd/m²) at 2,000 cd/m² and measuring the time for the current efficiency (cd/A) to decrease to 95%.

The measured life-spans of the organic light emitting diodes according to Examples 1 and 2 and Comparative Example 1 were calculated as relative values based on Comparative Example 1 and are shown in Table 1.

TABLE 1

| | Host | | | |
|---|---|---|---|---|
| | First | Second | Luminous | Life-span |
| No. | compound | compound | efficiency (%) | (%) |
| Example 1 | A-54 | D-72 | 105 | 162 |
| Example 2 | A-55 | D-72 | 105 | 158 |
| Comparative | A-54 | HT-1 | 100 | 100 |
| Example 1 | | | | |

Referring to Table 1, the organic light emitting diodes according to Examples 1 and 2 exhibited improved luminous efficiency and life-span characteristics compared to the organic light emitting diode according to Comparative Example 1.

By way of summation and review, examples of the organic optoelectronic device may include an organic photoelectric device, an organic light emitting diode, an organic solar cell, or an organic photoconductor drum.

Among them, organic light emitting diodes (OLEDs) are attracting much attention in recent years due to increasing demands for flat panel display devices. The organic light emitting diode may be a device that converts electrical energy into light, and the performance of the organic light emitting diode may be greatly influenced by an organic material between electrodes.

Some embodiments may provide a composition for an organic optoelectronic device that may achieve high efficiency and long life-span characteristics.

Some embodiments may provide an organic optoelectronic device including the composition for an organic optoelectronic device.

Some embodiments may provide a display device including the organic optoelectronic device.

An organic optoelectronic device having high efficiency and a long life-span may be realized.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A composition for an organic optoelectronic device, the composition comprising:
a first compound represented by Chemical Formula 1; and
a second compound represented by Chemical Formula 2: 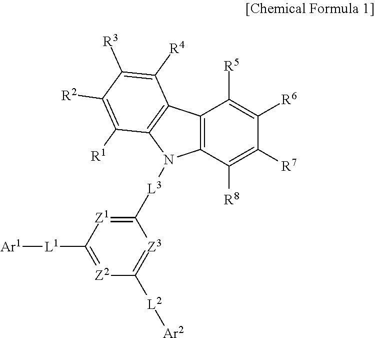
wherein, in Chemical Formula 1,
Z¹ to Z³ are each independently N or CR^{a}, provided that at least two of Z¹ to Z³ are N,
L¹ to L³ are each independently a single bond, a substituted or unsubstituted C6 to C20 arylene group or a substituted or unsubstituted divalent C2 to C20 heterocyclic group,
one of Ar¹ and Ar² is a substituted or unsubstituted carbazolyl group,
the other of Ar¹ and Ar² is a substituted or unsubstituted C6 to C30 aryl group,
R¹ to R⁸ and R^{a} are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, and
R¹ to R⁸ are each separately present or two adjacent ones are linked to form a ring, 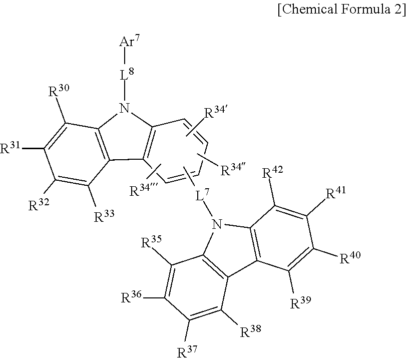
wherein, in Chemical Formula 2,
L⁷ and L⁸ are each independently a single bond or a substituted or unsubstituted C6 to C30 arylene group,
Ar⁷ is a substituted or unsubstituted C6 to C30 aryl group or a substituted or unsubstituted C2 to C30 heterocyclic group, and
R³⁰ to R³³, R^{34′}, R^{34″}, R^{34′″}, and R³⁵ to R⁴² are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen.

2. The composition for an organic optoelectronic device of claim 1, wherein:
L¹ and L² are each independently a single bond or a substituted or unsubstituted C6 to C20 arylene group, and
L³ is a single bond.

3. The composition for an organic optoelectronic device of claim 1, wherein the first compound is represented by Chemical Formula 1a: 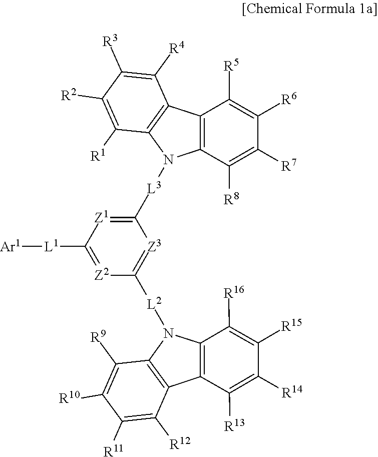
wherein, in Chemical Formula 1a,
Z¹ to Z³, L¹ to L³, and R¹ to R⁸ are defined the same as those of Chemical Formula 1,
Ar¹ is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, or a substituted or unsubstituted triphenylene group,
R⁹ to R¹⁶ are each independently hydrogen, deuterium, a substituted or unsubstituted C1 to C20 alkyl group, a substituted or unsubstituted C6 to C30 aryl group, a substituted or unsubstituted C2 to C30 heterocyclic group, a substituted or unsubstituted amine group, a substituted or unsubstituted silyl group, a cyano group, or a halogen, and
R⁹ to R¹⁶ are each separately present or two adjacent ones are linked to form a ring.

4. The composition for an organic optoelectronic device of claim 3, wherein:
L² is a substituted or unsubstituted phenylene group, a substituted or unsubstituted biphenylene group, a substituted or unsubstituted terphenylene group, or a substituted or unsubstituted naphthylene group, and
L³ is a single bond.

5. The composition for an organic optoelectronic device as claimed in claim 1, wherein the second compound is represented by one of Chemical Formulae 2a to 2d: 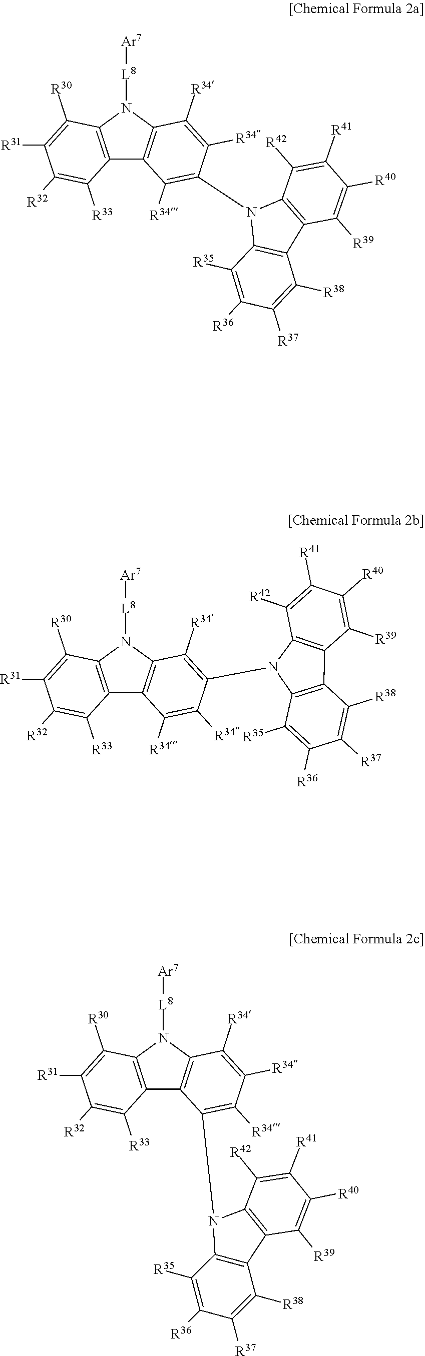 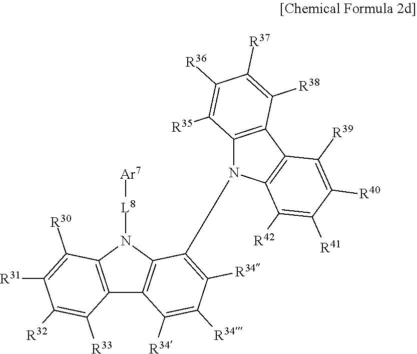
in Chemical Formulae 2a to 2d, L⁸, Ar⁷, R³⁰ to R³³, R^{34′}, R^{34″}, R^{34′″}, and R³⁵ to R⁴² are defined the same as those of Chemical Formula 2.

6. The composition for an organic optoelectronic device of claim 5, wherein:
L⁸ is a single bond, a substituted or unsubstituted phenylene group, a substituted or unsubstituted biphenylene group, or a substituted or unsubstituted terphenylene group, and
Ar⁷ is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylene group, a substituted or unsubstituted carbazolyl group, a substituted or unsubstituted benzothiophenyl group, a substituted or unsubstituted dibenzothiophenyl group, a substituted or unsubstituted benzofuranyl group, a substituted or unsubstituted dibenzofuranyl group, a substituted or unsubstituted benzosilolyl group, a substituted or unsubstituted dibenzosilolyl group, a substituted or unsubstituted fluorenyl group, or a substituted or unsubstituted silyl group.

7. The composition for an organic optoelectronic device of claim 5, wherein at least one of Ar⁷ and R³⁰ to R⁴² is a substituted or unsubstituted carbazolyl group.

8. The composition for an organic optoelectronic device of claim 1, wherein at least one of the first compound represented by Chemical Formula 1 and the second compound represented by Chemical Formula 2 is substituted with at least one deuterium.

9. The composition for an organic optoelectronic device of claim 1, wherein the first compound and the second compound are included in a weight ratio of about 10:90 to about 90:10.

10. An organic optoelectronic device, comprising:
an anode and a cathode facing each other; and
a light emitting layer between the anode and the cathode;
wherein the light emitting layer includes the composition for an organic optoelectronic device of claim 1.

11. The organic optoelectronic device of claim 10, wherein the light emitting layer further includes a fluorescent dopant, a phosphorescent sensitizer, or a combination thereof.

12. The organic optoelectronic device of claim 11, wherein the phosphorescent sensitizer is an organometallic compound, and the fluorescent dopant is a condensed polycyclic compound including boron, nitrogen, or a combination thereof.

13. The organic optoelectronic device of claim 10, wherein the light emitting layer emits light in a blue emission spectrum.

14. The organic optoelectronic device of claim 10, further comprising:
a hole transport layer between the anode and the light emitting layer, and
a hole transport auxiliary layer between the light emitting layer and the hole transport layer.

15. A display device comprising the organic optoelectronic device of claim 10.
